(19) United States
(12) Patent Application Publication
(10) Pub. No.: US 2025/0185635 A1
(43) Pub. Date: Jun. 12, 2025

(54) GENETICALLY MODIFIED NON-HUMAN ANIMALS WITH HUMANIZED IMMUNOGLOBULIN AND MHC LOCI

(71) Applicant: Biocytogen Pharmaceuticals (Beijing) Co., Ltd., Beijing (CN)

(72) Inventors: Yuelei Shen, Beijing (CN); Jiawei Yao, Beijing (CN); Huizhen Zhao, Beijing (CN); Jun Du, Beijing (CN); Ying Zhao, Beijing (CN)

(21) Appl. No.: 18/845,951

(22) PCT Filed: Mar. 21, 2023
(86) PCT No.: PCT/CN2023/082873

§ 371 Date: Sep. 11, 2024

(30) Foreign Application Priority Data
Mar. 21, 2022 (WO) PCT/CN2022/081924

(51) Int. Cl. A01K 67/0278; C07K 16/28; C12N 15/85; C12Q 1/6869

(52) U.S. Cl. CPC A01K 67/0278; C07K 16/2833; C12N 15/8509; C12Q 1/6869; A01K 2207/15; A01K 2217/072; A01K 2227/105; A01K 2267/01; A01K 2267/03; C12N 2015/8527

(57) ABSTRACT

Genetically modified animals and cells with humanized light chain immunoglobulin locus and/or humanized heavy chain immunoglobulin locus. The animals and cells can also express a human or chimeric (e.g., humanized) major histocompatibility complex (MHC) protein complex.

### CLAIM OF PRIORITY

This application claims the benefit of PCT Application No. PCT/CN2022/081924, filed on Mar. 21, 2022. The entire contents of the foregoing application are incorporated herein by reference.

### TECHNICAL FIELD

This disclosure relates to genetically modified animals and cells with humanized heavy chain immunoglobulin locus and/or humanized light chain immunoglobulin locus. The animals and cells can also express a human or chimeric major histocompatibility complex (MHC) protein complex.

### BACKGROUND

Major histocompatibility complex (MHC) class I and class II proteins play a pivotal role in the adaptive branch of the immune system. Both classes of proteins share the task of presenting peptides on the cell surface for recognition by T cells. Immunogenic peptide-MHC class I (pMHCI) complexes are presented on nucleated cells and are recognized by cytotoxic CD8+ T cells. In contrast, the presentation of pMHCII by antigen-presenting cells (e.g., dendritic cells (DCs), macrophages, or B cells) can activate CD4+ T cells, leading to the coordination and regulation of effector cells.

Human MHC protein complexes are highly polymorphic, and are different from animals' MHC. There is a need for to generate antibodies, particularly human or humanized antibodies, that can recognize the peptide-MHC complex for various therapeutic use.

### SUMMARY

The present disclosure relates to genetically modified animals and cells with humanized heavy chain and light chain immunoglobulin locus. In some embodiments, the animals and cells can also express a human or chimeric (e.g., humanized) major histocompatibility complex (MHC) protein complex. In some embodiments, the genetically modified animals have a limited set of human IGKV and IGKJ genes at the endogenous light chain immunoglobulin gene locus. In one aspect, the genetic modified animals as described herein can produce immunoglobulin light chain variable domains that can pair with a rather diverse family of heavy chain variable domains, including e.g., affinity matured or somatically mutated variable domains.

In one aspect, the disclosure is related to a genetically-modified non-human animal comprising at an endogenous heavy chain immunoglobulin gene locus, one or more human IGHV genes, one or more human IGHD genes, and one or more human IGHJ genes, in some embodiments, the human IGHV genes, the human IGHD genes, and the human IGHJ genes are operably linked and can undergo VDJ rearrangement, in some embodiments, the animal expresses a fusion protein comprising β2 microglobulin (B2M) and a human or humanized major histocompatibility complex (MHC) α chain.

In some embodiments, the animal comprises at least 150 human IGHV genes selected from Table 1, at least 20 human IGHD genes selected from Table 2, and at least 5 human IGHJ genes selected from Table 3. In some embodiments, the animal comprises all human IGHV genes, all human IGHD genes, and all human IGHJ genes at the endogenous heavy chain immunoglobulin gene locus of human chromosome 14 of a human subject. In some embodiments, the animal comprises all human IGHV genes, all human IGHD genes, and all human IGHJ genes at the endogenous heavy chain immunoglobulin gene locus of human chromosome 14 of a human cell. In some embodiments, the animal comprises a disruption in the animal's endogenous heavy chain immunoglobulin gene locus. In some embodiments, the animal is a mouse and the disruption in the animal's endogenous heavy chain immunoglobulin gene locus comprises a deletion of one or more mouse IGHV genes in Table 4, one or more mouse IGHD genes in Table 5, and/or one or more mouse IGHJ genes in Table 6. In some embodiments, the animal is a mouse and the disruption in the animal's endogenous heavy chain immunoglobulin gene locus comprises a deletion of a contiguous sequence starting from mouse IGHV1-85 gene to mouse IGHJ4 gene. In some embodiments, the animal comprises one or more endogenous IGHM, IGHδ, IGHG3, IGHG1, IGHG2b, IGHG2a, IGHE, and IGHA genes. In some embodiments, the animal comprises an unmodified human sequence derived from a human heavy chain immunoglobulin gene locus, in some embodiments, the unmodified human sequence is at least 800 kb. In some embodiments, the animal comprises an unmodified human sequence derived from a human heavy chain immunoglobulin gene locus starting from human IGHV(III)-82 to human IGHV1-2. In some embodiments, the animal comprises an unmodified human sequence derived from a human heavy chain immunoglobulin gene locus starting from human IGHV(III)-82 to human IGHV6-1. In some embodiments, the animal comprises an unmodified human sequence derived from a human heavy chain immunoglobulin gene locus starting from human IGHD1-1 to human IGHJ6. In some embodiments, the animal comprises an unmodified human sequence derived from a human heavy chain immunoglobulin gene locus starting from human IGHV(III)-82 to human IGHJ6. In some embodiments, the animal is homozygous with respect to the heavy chain immunoglobulin gene locus. In some embodiments, the animal is heterozygous with respect to the heavy chain immunoglobulin gene locus. In some embodiments, the animal further comprises at an endogenous light chain immunoglobulin gene locus, one or more human IGKV genes, and one or more human IGKJ genes. In some embodiments, the animal comprises a disruption in the animal's endogenous lambda light chain immunoglobulin gene locus. In some embodiments, the animal is a rodent (e.g., a mouse).

In one aspect, the disclosure is related to a genetically-modified non-human animal comprising at an endogenous light chain immunoglobulin gene locus, one or more human IGKV genes and one or more human IGKJ genes, in some embodiments, the animal expresses a fusion protein comprising β2 microglobulin (B2M) and a human or humanized major histocompatibility complex (MHC) α chain. In some embodiments, the animal comprises all human IGKV genes in Table 7, and all human IGKJ genes in Table 8. In some embodiments, the animal comprises an unmodified sequence derived from a human light chain immunoglobulin gene locus starting from human IGKV3D-7 to human IGKJ5. In some embodiments, the animal comprises a disruption in the animal's endogenous light chain immunoglobulin gene locus. In some embodiments, the animal is a mouse and the disruption in the animal's endogenous light chain immunoglobulin gene locus comprises a deletion of one or more mouse IGKV genes in Table 9 and one or more mouse IGKJ genes in Table 10. In some embodiments, the animal is a mouse and the disruption in the animal's endogenous light chain immunoglobulin gene locus comprises a deletion of a sequence starting from mouse IGKV2-137 to mouse IGKJ5. In some embodiments, the animal comprises an endogenous IGKC. In some embodiments, the animal is homozygous with respect to the light chain immunoglobulin gene locus. In some embodiments, the animal is heterozygous with respect to the light chain immunoglobulin gene locus. In some embodiments, the animal further comprises at an endogenous heavy chain immunoglobulin gene locus, one or more human IGHV genes, one or more human IGHD genes, and one or more human IGHJ genes. In some embodiments, the animal comprises a disruption in the animal's endogenous lambda light chain immunoglobulin gene locus. In some embodiments, the animal is a rodent (e.g., a mouse).

In one aspect, the disclosure is related to a genetically-modified non-human animal comprising at the endogenous light chain immunoglobulin locus, an exogenous light chain variable region gene sequence, in some embodiments, the exogenous light chain variable region gene sequence comprises no more than three human IGKV genes and no more than two human IGKJ genes, in some embodiments, the no more than three human IGKV genes and the no more than two human IGKJ genes are operably linked to an endogenous light chain constant domain gene, in some embodiments, the animal expresses a fusion protein comprising β2 microglobulin (B2M) and a human or humanized major histocompatibility complex (MHC) α chain. In some embodiments, the no more than three human IGKV genes are selected from Table 7, and the no more than two human IGKJ genes are selected from Table 8. In some embodiments, the exogenous light chain variable region gene sequence comprises one human IGKV gene and one human IGKJ gene. In some embodiments, the exogenous light chain variable region gene sequence further comprises a human IGKJ 3′-UTR sequence. In some embodiments, the exogenous light chain variable region gene in one or more cells of the animal can subject to somatic hypermutations. In some embodiments, the somatic hypermutations can result in up to one, two, or three amino acid changes in light chain variable regions in the one or more cells of the animal. In some embodiments, exogenous light chain variable region gene sequence comprises one human IGKV gene and one human IGKJ gene, in some embodiments, the human IGKV gene is selected from the group consisting of IGKV3-20, IGKV3-11, and IGKV1-39, in some embodiments, the human IGKV gene and the human IGKJ gene are operably linked. In some embodiments, the human IGKV gene is IGVK3-11. In some embodiments, the human IGKJ gene is selected from the group consisting of IGKJ1 and IGKJ4. In some embodiments, the human IGKV gene is IGKV1-39 and the human IGKJ gene is IGKJ4. In some embodiments, the human IGKV gene is IGKV3-11 and the human IGKJ gene is IGKJ1. In some embodiments, the human IGKV gene is IGKV3-20 and the human IGKJ gene is IGKJ1. In some embodiments, the animal further comprises a promoter sequence that is operably linked to the human IGKV gene, in some embodiments, the promoter sequence is within 2500 or 3000 bp of the human IGKV gene. In some embodiments, the promoter is an IGKV3-20 promoter, an IGKV3-11 promoter, or an IGKV1-39 promoter. In some embodiments, the animal comprises a disruption in the animal's endogenous light chain immunoglobulin gene locus. In some embodiments, the animal is a mouse and the disruption in the animal's endogenous light chain immunoglobulin gene locus comprises a deletion of one or more mouse IGKV genes in Table 9 and one or more mouse IGKJ genes in Table 10. In some embodiments, the animal is a mouse and the disruption in the animal's endogenous light chain immunoglobulin gene locus comprises a deletion of a sequence starting from mouse IGKV2-137 to mouse IGKJ5. In some embodiments, the animal comprises an endogenous IGKC. In some embodiments, the animal further comprises a kappa intronic enhancer 5′ with respect to the endogenous IGKC and/or a kappa 3′ enhancer. In some embodiments, the human light chain variable region is a rearranged sequence. In some embodiments, the animal is homozygous with respect to the light chain immunoglobulin gene locus. In some embodiments, the animal is heterozygous with respect to the light chain immunoglobulin gene locus. In some embodiments, the animal comprises a disruption in the animal's endogenous lambda light chain immunoglobulin gene locus. In some embodiments, the animal is a rodent (e.g., a mouse). In some embodiments, the animal further comprises at an endogenous heavy chain immunoglobulin gene locus, one or more human IGHV genes, one or more human IGHD genes, and one or more human IGHKJ genes, in some embodiments, the human IGHV genes, the human IGHD genes, and the human IGHJ genes are operably linked and can undergo VDJ rearrangement. In some embodiments, the animal comprises at least 150 human IGHV genes selected from Table 1, at least 20 human IGHD genes selected from Table 2, and at least 5 human IGHJ genes selected from Table 3. In some embodiments, the animal comprises all human IGHV genes, all human IGHD genes, and all human IGHJ genes at the endogenous heavy chain immunoglobulin gene locus of human chromosome 14 of a human subject. In some embodiments, the animal comprises all human IGHV genes, all human IGHD genes, and all human IGHJ genes at the endogenous heavy chain immunoglobulin gene locus of human chromosome 14 of a human cell. In some embodiments, the animal comprises an unmodified human sequence derived from a human heavy chain immunoglobulin gene locus, in some embodiments, the unmodified human sequence is at least 800 kb. In some embodiments, the animal comprises an unmodified human sequence derived from a human heavy chain immunoglobulin gene locus starting from human IGHV(III)-82 to human IGHV1-2. In some embodiments, the animal comprises an unmodified human sequence derived from a human heavy chain immunoglobulin gene locus starting from human IGHV(III)-82 to human IGHV6-1. In some embodiments, the animal comprises an unmodified human sequence derived from a human heavy chain immunoglobulin gene locus starting from human IGHD1-1 to human IGHJ6. In some embodiments, the animal comprises an unmodified human sequence derived from a human heavy chain immunoglobulin gene locus starting from human IGHV(III)-82 to human IGHJ6.

In some embodiments, the animal lacks an endogenous immunoglobulin heavy chain variable region locus that is capable of rearranging and forming a nucleic acid sequence that encodes an endogenous heavy chain variable domain (e.g., a mouse heavy chain variable domain). In some embodiments, the animal lacks an endogenous immunoglobulin light chain variable region locus that is capable of rearranging and forming a nucleic acid sequence that encodes an endogenous light chain variable domain (e.g., a mouse light chain variable domain). In some embodiments, the animal can produce a humanized antibody.

In some embodiments, the genome of the animal comprises at least one chromosome comprising a sequence encoding the fusion protein. In some embodiments, the fusion protein comprises a human or humanized B2M protein. In some embodiments, the MHC α chain is a MHC class I α chain. In some embodiments, the MHC α chain is a chimeric MHC α chain. In some embodiments, the MHC α chain is a human HLA protein (e.g., HLA-A, HLA-B, or HLA-C). In some embodiments, the MHC α chain is a human HLA/mouse H-2 chimeric molecule, in some embodiments, the human HLA is selected from the group consisting of HLA-A, HLA-B, HLA-C, and in some embodiments, the mouse H-2 is selected from the group consisting of H-2K, H-2D, and H-2L. In some embodiments, the MHC α chain is a human HLA/mouse H2-D1 chimeric molecule. In some embodiments, the fusion protein comprises a human B2M protein and a chimeric MHC α chain comprising human HLA α1 and α2 domains. In some embodiments, the chimeric MHC α chain further comprises a mouse H2-D1 α3 domain. In some embodiments, the sequence encoding the fusion protein is operably linked to an endogenous regulatory element (e.g., a promoter) at the endogenous β2 microglobulin (B2M) gene locus in the at least one chromosome. In some embodiments, the human HLA is human HLA-A*0101, HLA-A*0201, HLA-A*0301, HLA-A*0302, HLA-A*1101, HLA-A*2402, HLA-A*2901, HLA-A*3101, HLA-A*3201, HLA-A*3301, HLA-A*3303, HLA-B*4402, HLA-B*0702, HLA-C*0702, HLA-C*0102, HLA-C*0701, HLA-C*0401, HLA-C*0801, or HLA-C*0802.

In some embodiments, the fusion protein comprises (a) a human B2M; and (b) a human HLA (e.g., HLA-A, HLA-B, or HLA-C). In some embodiments, the human B2M and the human HLA are linked via a linker peptide sequence. In some embodiments, the fusion protein comprises (a) a human B2M; and (b) a chimeric MHC α chain. In some embodiments, the human B2M and the chimeric MHC α chain are linked via a linker peptide sequence. In some embodiments, the chimeric MHC α chain comprises human HLA α1 and α2 domains. In some embodiments, the chimeric MHC α chain further comprises a human HLA α3 domain. In some embodiments, the chimeric MHC α chain further comprises a MHC α3 domain endogenous to the animal and/or a MHC cytoplasmic region endogenous to the animal. In some embodiments, the chimeric MHC α chain comprises a α3 domain, a connecting peptide, a transmembrane region, and a cytoplasmic region of an endogenous MHC. In some embodiments, the animal is a mouse, and the chimeric MHC α chain comprises a α3 domain, a connecting peptide, a transmembrane region, and a cytoplasmic region of mouse H2-D1. In some embodiments, the fusion protein further comprises a signal peptide of the human HLA (e.g., at the N-terminus of the fusion protein). In some embodiments, the animal is heterozygous with respect to the sequence encoding the fusion protein. In some embodiments, the animal is homozygous with respect to the sequence encoding the fusion protein.

In one aspect, the disclosure is related to a cell obtained from the animal as described herein. In some embodiments, the cell is a B cell that expresses a chimeric immunoglobulin heavy chain comprising an immunoglobulin heavy chain variable domain that is derived from a rearrangement of one or more human IGHV genes, one or more human IGHD genes, and one or more human IGHJ genes, in some embodiments, the immunoglobulin heavy chain variable domain is operably linked to a non-human heavy chain constant region. In some embodiments, the cell is a B cell that expresses a chimeric immunoglobulin light chain comprising an immunoglobulin light chain variable domain that is derived from a rearrangement of one or more human IGKV genes and one or more human IGKJ genes, and in some embodiments, the immunoglobulin light chain variable domain is operably linked to a non-human light chain constant region. In some embodiments, the cell is an embryonic stem (ES) cell.

In one aspect, the disclosure is related to a method of making an antibody that specifically binds to an antigen peptide-MHC complex, the method comprising exposing the animal as described herein to the antigen peptide-MHC complex comprising the antigen peptide. In some embodiments, the method further comprises producing a hybridoma from a cell collected from the animal; and collecting or analyzing the antibody produced by the hybridoma. In some embodiments, the method further comprises sequencing the genome of the hybridoma. In some embodiments, the antigen peptide is an intracellular antigen peptide (e.g., any one of the antigen peptides listed in Table 16).

In one aspect, the disclosure is related to a method of obtaining a nucleic acid that encodes an antibody binding domain that specifically binds to an antigen peptide-MHC complex, the method comprising exposing the animal as described herein to the antigen peptide-MHC complex comprising the antigen peptide; and sequencing nucleic acids encoding human heavy and light chain immunoglobulin variable regions in a cell that expresses a hybrid antibody that specifically binds to the antigen peptide-MHC complex.

In one aspect, the disclosure is related to a method of obtaining a sample, the method comprising exposing the animal as described herein to an antigen peptide-MHC complex; and collecting the sample from the animal. In some embodiments, the sample is a spleen tissue, a lymphoid tissue, spleen cell, or a B cell.

In one aspect, the disclosure is related to a method of screening an antibody that specifically binds to an antigen peptide-MHC complex, the method comprising exposing the animal as described herein to the antigen peptide-MHC complex comprising an antigen peptide of interest; producing a hybridoma from a cell collected from the animal; collecting or analyzing antibodies produced by the hybridoma; incubating the antibodies with cells presenting the antigen peptide of interest, cells presenting a control antigen peptide and/or cells do not present any antigen peptide; and determining that the antibodies can specifically bind to cells presenting the antigen peptide of interest, and optionally determining that the antibodies cannot bind to the cells presenting the control antigen peptide or the cells that do not present any antigen peptide. In some embodiments, determining that the antibodies can specifically bind to cells presenting the antigen peptide of interest is determined by flow cytometry.

In one aspect, the disclosure is related to a method of screening an antibody that specifically binds to an antigen peptide-MHC complex, the method comprising exposing the animal as described herein to the antigen peptide-MHC complex comprising an antigen peptide of interest; and sequencing nucleic acids encoding human heavy and light chain immunoglobulin variable regions in the cell that expresses an antibody that specifically binds to the antigen peptide-MHC complex. In some embodiments, the method described herein further comprises (a) expressing an antibody comprising the encoding human heavy and light chain immunoglobulin variable regions; (b) incubating the antibody with cells presenting the antigen peptide of interest; and (c) determining that the antibody can specifically bind to cells presenting the antigen peptide of interest. In some embodiments, the method described herein further comprises (a) incubating the antibody with cells presenting a control antigen peptide and/or cells do not present any antigen peptide; and (b) determining that the antibody cannot bind to the cells presenting the control antigen peptide or the cells that do not present any antigen peptide.

In some embodiments, the genetically-modified, non-human animal is heterozygous with respect to the modified B2M locus. In some embodiments, the genetically-modified, non-human animal is homozygous with respect to the modified B2M locus.

In some embodiments, the genetically-modified, non-human animal is heterozygous with respect to the modified MHC locus. In some embodiments, the genetically-modified, non-human animal is homozygous with respect to the modified MCH locus.

In some aspects, the disclosure relates to a genetically-modified, non-human animal comprising at an endogenous heavy chain immunoglobulin gene locus, one or more human IGHV genes, one or more human IGHD genes, and one or more human IGHJ genes. In some embodiments, the human IGHV genes, the human IGHD genes, and the human IGHJ genes are operably linked and can undergo VDJ rearrangement.

In some embodiments, the animal comprises about or at least 150, 151, 152, 153, 154, 155, 156, 157, 158, 159, 160 or 161 human IGHV genes selected from Table 1, about or at least 20, 21, 22, 23, 24, 25, 26, or 27 human IGHD genes selected from Table 2, and about or at least 5, 6, 7, 8, or 9 human IGHJ genes selected from Table 3. In some embodiments, the animal comprises all human IGHV genes in Table 1 except IGHV2-10, IGHV3-9, and IGHV1-8, all human IGHD genes in Table 2, and all human IGHJ genes in Table 3. In some embodiments, the animal comprises all human IGHV genes in Table 1 except IGHV5-10-1 and IGHV3-64D, all human IGHD genes in Table 2, and all human IGHJ genes in Table 3. In some embodiments, the animal comprises all human IGHV genes, all human IGHD genes, and all human IGHJ genes at the endogenous heavy chain immunoglobulin gene locus of human chromosome 14 of a human subject. In some embodiments, the animal comprises all human IGHV genes, all human IGHD genes, and all human IGHJ genes at the endogenous heavy chain immunoglobulin gene locus of human chromosome 14 of a human cell (e.g., a somatic cell, a cultured cell, a non-immune cell, a cell without any V(D)J rearrangement).

In some embodiments, the animal comprises an unmodified human sequence derived from a human heavy chain immunoglobulin gene locus. In some embodiments, the unmodified human sequence is about or at least 10, 20, 30, 40, 50, 60, 70, 80, 90, 100, 200, 300, 400, 500, 600, 700, 800, 900, or 1000 kb.

In some aspects, the disclosure relates to a genetically-modified animal comprising at an endogenous heavy chain immunoglobulin gene locus, a first sequence comprising one or more human IGHV genes; a second sequence comprising an endogenous sequence; and a third sequence comprising one or more human IGHD genes, and one or more human IGHJ genes, wherein the first sequence, the second sequence, and the third sequence are operably linked.

In some embodiments, the first sequence comprises about or at least 150, 151, 152, 153, 154, 155, 156, 157, 158, 159, 160 or 161 human IGHV genes selected from Table 1. In some embodiments, the first sequence comprises about or at least 20, 21, 22, 23, 24, 25, 26, or 27 human IGHD genes selected from Table 2.

In some embodiments, the first sequence is an unmodified sequence derived from a human heavy chain immunoglobulin gene locus. In some embodiments, the first sequence is about or at least 10, 20, 30, 40, 50, 60, 70, 80, 90, 100, 200, 300, 400, 500, 600, 700, 800, 900, or 1000 kb.

In some embodiments, the second sequence comprises an endogenous sequence that is about or at least 1, 2, 3, 4, 5, 6, 7, 8, 9, 10, 11, 12, 13, 14, 15, 16, 17, 18, 19, or 20 kb.

In some embodiments, the third sequence comprises about or at least 20, 21, 22, 23, 24, 25, 26, or 27 human IGHD genes selected from Table 2. In some embodiments, the third sequence comprises about or at least 5, 6, 7, 8, or 9 human IGHJ genes selected from Table 3. In some embodiments, the third sequence comprises all human IGHD genes in Table 2, and all human IGHJ genes in Table 3.

In some embodiments, the third sequence is an unmodified sequence derived from a human heavy chain immunoglobulin gene locus. In some embodiments, the third sequence is about or at least 10, 20, 30, 40, 50, 60, 70, 80, 90 or 100 kb.

In some embodiments, the animal comprises a disruption in the animal's endogenous heavy chain immunoglobulin gene locus.

In some embodiments, the animal is a mouse and the disruption in the animal's endogenous heavy chain immunoglobulin gene locus comprises a deletion of one or more mouse IGHV genes in Table 4, one or more mouse IGHD genes in Table 5, and one or more mouse IGHJ genes in Table 6.

In some embodiments, the animal is a mouse and the disruption in the animal's endogenous heavy chain immunoglobulin gene locus comprises a deletion of a sequence starting from mouse IGHV1-85 to mouse IGHJ4.

In some embodiments, the animal comprises one or more endogenous genes selected from the group consisting of IGHM, IGHδ, IGHG3, IGHG1, IGHG2b, IGHG2a, IGHE, and IGHA genes.

In some embodiments, the animal is homozygous with respect to the heavy chain immunoglobulin gene locus. In some embodiments, the animal is heterozygous with respect to the heavy chain immunoglobulin gene locus.

In some aspects, the disclosure relates to a genetically-modified, non-human animal comprising at an endogenous light chain immunoglobulin gene locus, one or more human IGKV genes and one or more human IGKJ genes.

In some embodiments, the animal comprises about or at least 65, 66, 67, 68, 69, 70, 71, 72, 73, 74, 75, or 76 human IGKV genes in Table 7, and/or comprises about or at least 1, 2, 3, 4, or 5 human IGKJ genes in Table 8.

In some embodiments, the animal comprises an unmodified sequence derived from a human light chain immunoglobulin gene locus starting from human IGKV3D-7 to human IGKJ5.

In some embodiments, the animal comprises a disruption in the animal's endogenous light chain immunoglobulin gene locus.

In some embodiments, the animal comprises all human IGKV genes, and all human IGKJ genes at the endogenous kappa chain immunoglobulin gene locus of human chromosome 2 of a human subject. In some embodiments, the animal comprises all human IGKV genes, and all human IGKJ genes at the endogenous heavy chain immunoglobulin gene locus of human chromosome 2 of a human cell (e.g., a somatic cell, a cultured cell, a non-immune cell, a cell without any V(D)J rearrangement).

In some aspects, the disclosure relates to a genetically-modified, non-human animal whose genome comprises an endogenous heavy chain immunoglobulin locus comprising: a replacement of one or more endogenous IGHV, endogenous IGHD, and endogenous IGHJ genes with one or more human IGHV, human IGHD, and human IGHJ genes. In some embodiments, human IGHV, human IGHD, and human IGHJ genes are operably linked to one or more of endogenous genes selected from the group consisting of IGHM, IGHδ, IGHG, IGHE, and IGHA genes.

In some embodiments, one or more endogenous IGHV, endogenous IGHD, and endogenous IGHJ genes are replaced by about or at least 150, 151, 152, 153, 154, 155, 156, 157, 158, 159, 160 or 161 human IGHV genes in Table 1, about or at least 20, 21, 22, 23, 24, 25, 26, or 27 human IGHD genes in Table 2, and about or at least 5, 6, 7, 8, or 9 human IGHJ genes in Table 3.

In some embodiments, the animal is a mouse, and about or at least 180 mouse IGHV genes in Table 4, all mouse IGHD genes in Table 5, and all mouse IGHJ genes in Table 6 are replaced.

In some aspects, the disclosure relates to a genetically-modified, non-human animal whose genome comprises an endogenous light chain immunoglobulin locus comprising: a replacement of one or more endogenous IGKV and endogenous IGKJ genes with one or more human IGKV and human IGKJ genes. In some embodiments, the human IGKV and human IGKJ genes are operably linked to an endogenous IGKC gene.

In some embodiments, one or more endogenous IGKV and endogenous IGKJ genes are replaced by about or at least 65, 66, 67, 68, 69, 70, 71, 72, 73, 74, 75, or 76 human IGKV genes in Table 7, and about or at least 1, 2, 3, 4, or 5 human IGKJ genes in Table 8.

In some embodiments, the animal is a mouse, and all mouse IGKV genes in Table 9 and all mouse IGKJ genes in Table 10 are replaced.

In some aspects, the disclosure relates to a method of modifying genome of a cell, the method comprising modifying a human chromosome; introducing the modified human chromosome into a cell of the animal; and inducing recombination between the modified human chromosome and an endogenous chromosome, thereby replacing one or more endogenous genes with one or more human genes.

In some embodiments, the modified human chromosome comprises two or more exogenous recombination sites.

In some embodiments, the endogenous chromosome comprises two or more exogenous recombination sites.

In some embodiments, about or at least 150, 151, 152, 153, 154, 155, 156, 157, 158, 159, 160 or 161 human IGHV genes selected from Table 1, about or at least 20, 21, 22, 23, 24, 25, 26, or 27 human IGHD genes selected from Table 2, and about or at least 5, 6, 7, 8, or 9 human IGHJ genes selected from Table 3 are integrated into the endogenous chromosome by recombination.

In some embodiments, about or at least 65, 66, 67, 68, 69, 70, 71, 72, 73, 74, 75, or 76 human IGKV genes in Table 7, and about or at least 1, 2, 3, 4, or 5 human IGKJ genes in Table 8 are integrated into the endogenous chromosome by recombination.

In some embodiments, a human sequence is integrated into the endogenous chromosome by recombination, and the human sequence is about or at least 10, 20, 30, 40, 50, 60, 70, 80, 90, 100, 200, 300, 400, 500, 600, 700, 800, 900, or 1000 kb.

In one aspect, the disclosure provides a method of making an antibody that specifically binds to an antigen. The method involves obtaining a nucleic acid sequence encoding human heavy and light chain immunoglobulin variable regions in a cell that expresses a hybrid antibody that specifically binds to the antigen, wherein the cell is obtained by exposing the animal as described herein to an antigen-MHC complex comprising the antigen; operably linking the nucleic acid encoding the human heavy chain immunoglobulin variable region with a nucleic acid encoding a human heavy chain immunoglobulin constant region and the nucleic acid encoding the human light chain immunoglobulin variable region with a nucleic acid encoding a human light chain immunoglobulin constant region; and expressing the nucleic acid in a cell, thereby obtaining the antibody.

In one aspect, the disclosure provides a method of making an antibody that specifically binds to an antigen. The method involves exposing the animal as described herein to an antigen-MHC complex comprising the antigen; obtaining the sequence of (e.g. by sequencing) nucleic acids encoding human heavy and light chain immunoglobulin variable regions in a cell that expresses a hybrid antibody that specifically binds to the antigen; and operably linking in a cell the nucleic acid encoding the human heavy chain immunoglobulin variable region with a nucleic acid encoding a human heavy chain immunoglobulin constant region and the nucleic acid encoding the human light chain immunoglobulin variable region with a nucleic acid encoding a human light chain immunoglobulin constant region.

In one aspect, the disclosure provides a genetically-modified, non-human animal comprising at the endogenous light chain immunoglobulin locus, an exogenous light chain variable region gene sequence. In some embodiments, the exogenous light chain variable region gene sequence comprises no more than three human IGKV genes and no more than two human IGKJ genes. In some embodiments, the no more than three human IGKV genes and the no more than two human IGKJ genes are operably linked to an endogenous light chain constant domain gene.

In some embodiments, the animal comprises IGHV(III)-82, IGHV7-81, IGHV4-80, IGHV3-79, IGHV(II)-78-1, IGHV5-78, IGHV7-77, IGHV(III)-76-1, IGHV3-76, IGHV3-75, and IGHV(II)-74-1. In some embodiments, the animal comprises IGHV5-10-1 and IGHV3-64D.

In some embodiments, the animal further comprises at an endogenous heavy chain immunoglobulin gene locus, a first sequence comprising one or more human IGHV genes; a second sequence comprising an endogenous sequence; and a third sequence comprising one or more human IGHD genes, and one or more human IGHJ genes. In some embodiments, the first sequence, the second sequence, and the third sequence are operably linked.

In some embodiments, the first sequence comprises at least 150 human IGHV genes selected from Table 1. In some embodiments, the first sequence comprises at least 20 human IGHD genes selected from Table 2. In some embodiments, the first sequence is an unmodified sequence derived from a human heavy chain immunoglobulin gene locus. In some embodiments, the first sequence is at least 800 kb. In some embodiments, the second sequence comprises an endogenous sequence that is at least 3 kb. In some embodiments, the third sequence comprises at least 20 human IGHD genes selected from Table 2, and at least 5 human IGHJ genes selected from Table 3.

In some embodiments, the third sequence comprises all human IGHD genes in Table 2, and all human IGHJ genes in Table 3. In some embodiments, the third sequence is an unmodified sequence derived from a human heavy chain immunoglobulin gene locus. In some embodiments, the third sequence is at least 50 kb. In some embodiments, the animal comprises a disruption in the animal's endogenous heavy chain immunoglobulin gene locus.

In some embodiments, the animal is a mouse and the disruption in the animal's endogenous heavy chain immunoglobulin gene locus comprises a deletion of one or more mouse IGHV genes in Table 4, one or more mouse IGHD genes in Table 5, and one or more mouse IGHJ genes in Table 6. In some embodiments, the animal is a mouse and the disruption in the animal's endogenous heavy chain immunoglobulin gene locus comprises a deletion of a sequence starting from mouse IGHV1-85 to mouse IGHJ4. In some embodiments, the animal comprises one or more endogenous IGHM, IGHδ, IGHG3, IGHG1, IGHG2b, IGHG2a, IGHE, and IGHA genes.

In some embodiments, the animal is homozygous with respect to the heavy chain immunoglobulin gene locus. In some embodiments, the animal is heterozygous with respect to the heavy chain immunoglobulin gene locus.

In one aspect, the disclosure provides a genetically-modified, non-human animal whose genome comprises an endogenous light chain immunoglobulin locus comprising: a replacement of one or more endogenous IGKV with one or more human IGKV genes selected from Table 7; and a replacement of one or more endogenous IGKJ genes with one or more human IGKJ genes selected from Table 8. In some embodiments, the human IGKV gene and the human IGKJ gene are operably linked to an endogenous IGKC gene.

In some embodiments, the one or more human IGKV genes are selected from the group consisting of IGKV3-20, IGKV3-11, and IGKV1-39. In some embodiments, the human IGKV gene is IGKV3-11. In some embodiments, the one or more human IGKJ genes are selected from the group consisting of IGKJ1 and IGKJ4. In some embodiments, the animal further comprises an insertion of a human IGKJ 3′-UTR sequence. In some embodiments, the human IGKV gene is IGKV1-39 and the human IGKJ gene is IGKJ4. In some embodiments, the human IGKV gene is IGKV3-11 and the human IGKJ gene is IGKJ1. In some embodiments, the human IGKV gene is IGKV3-20 and the human IGKJ gene is IGKJ1. In some embodiments, all endogenous IGKV genes are replaced by the one or more human IGKV genes. In some embodiments, all endogenous IGKJ genes are replaced by the one or more human IGKJ genes.

In some embodiments, the animal further comprises a promoter sequence before the human IGKV gene. In some embodiments, the promoter sequence is within 3000 bp of the human IGKV gene. In some embodiments, the animal further comprises a kappa intronic enhancer at the 5′ of the endogenous IGKC. In some embodiments, the animal further comprises a kappa 3′ enhancer.

In some embodiments, the genome of the animal further comprises an endogenous heavy chain immunoglobulin locus comprising: a replacement of one or more endogenous IGHV, endogenous IGHD, and endogenous IGHJ genes with one or more human IGHV, human IGHD, and human IGHJ genes. In some embodiments, the one or more human IGHV, human IGHD, and human IGHJ genes are operably linked to one or more of endogenous IGHM, IGHδ, IGHG, IGHE, and IGHA genes. In some embodiments, the one or more endogenous IGHV, endogenous IGHD, and endogenous IGHJ genes are replaced by at least 150 human IGHV genes in Table 1, at least 20 human IGHD genes in Table 2, and at least 5 human IGHJ genes in Table 3.

In some embodiments, the animal is a mouse, and at least 180 mouse IGHV genes in Table 4, all mouse IGHD genes in Table 5, and all mouse IGHJ genes in Table 6 are replaced.

In some embodiments, the animal lacks an endogenous immunoglobulin heavy chain variable region locus that is capable of rearranging and forming a nucleic acid sequence that encodes an endogenous heavy chain variable domain (e.g., a mouse heavy chain variable domain). In some embodiments, the animal lacks an endogenous immunoglobulin light chain variable region locus that is capable of rearranging and forming a nucleic acid sequence that encodes an endogenous light chain variable domain (e.g., a mouse light chain variable domain).

In some embodiments, the animal can produce a humanized antibody. In some embodiments, the antibody comprises a light chain variable region encoded by the IGKV gene and IGKJ gene.

In one aspect, the disclosure provides a cell obtained from the animal as described herein. In some embodiments, the cell is a B cell that expresses a chimeric immunoglobulin light chain comprising an immunoglobulin light chain variable domain that is encoded by a human IGKV gene selected from the group consisting of IGKV3-20, IGKV3-11, and IGKV1-39, and a human IGKJ gene selected from the group consisting of IGKJ1 and IGKJ4. In some embodiments, the immunoglobulin light chain variable domain is operably linked to a non-human light chain constant region.

In some embodiments, the B cell that expresses a chimeric immunoglobulin heavy chain comprising an immunoglobulin heavy chain variable domain that is derived from a rearrangement of one or more human IGHV genes, one or more human IGHD genes, and one or more human IGHJ genes. In some embodiments, the immunoglobulin heavy chain variable domain is operably linked to a non-human heavy chain constant region.

In some embodiments, the cell is an embryonic stem (ES) cell.

In one aspect, the disclosure provides a method of making an antibody that specifically binds to an antigen, the method comprising exposing the animal as described herein to an antigen-MHC complex comprising the antigen; sequencing nucleic acids encoding human heavy and light chain immunoglobulin variable regions in a cell that expresses a hybrid antibody that specifically binds to the antigen; and operably linking in a cell the nucleic acid encoding the human heavy chain immunoglobulin variable region with a nucleic acid encoding a human heavy chain immunoglobulin constant region and the nucleic acid encoding the human light chain immunoglobulin variable region with a nucleic acid encoding a human light chain immunoglobulin constant region.

In one aspect, the disclosure provides a method of making an antibody that specifically binds to a protein of interest, the method comprising exposing the animal as described herein to the protein of interest, e.g., peptide-MHC complex; and sequencing nucleic acids encoding human heavy and light chain immunoglobulin variable regions in a cell that expresses an antibody that specifically binds to the protein of interest.

In some embodiments, a gene encoding the endogenous protein is disrupted in the animal. In some embodiments, the gene encoding the endogenous protein is knocked out.

In some embodiments, the endogenous protein is at least 80%, 90%, 95% homologous to the protein of interest. In some embodiments, the endogenous protein is at least 80%, 90%, or 95% identical to the protein of interest.

The disclosure also relates to an offspring of the non-human mammal.

In some embodiments, the non-human mammal is a rodent. In some embodiments, the non-human mammal is a mouse.

The disclosure also provides to a cell including the targeting vector as described herein.

The disclosure also relates to a cell (e.g., a stem cell, an embryonic stem cell, an immune cell, a B cell, a T cell, or a hybridoma) or a cell line, or a primary cell culture thereof derived from the non-human mammal or an offspring thereof. The disclosure further relates to the tissue, organ or a culture thereof derived from the non-human mammal or an offspring thereof.

The disclosure further relates to the use of the non-human mammal or an offspring thereof, the animal model generated through the method as described herein in the development of a product related to an immunization processes, the manufacture of a human antibody, or the model system for a research in pharmacology, immunology, microbiology and medicine.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing a non-limiting MHC humanization strategy.

FIG. 2A is a schematic diagram showing humanized mouse B2M gene locus. Mouse B2M gene coding region was replaced with a nucleic acid sequence encoding the signal peptide of human HLA-A, human B2M protein, a portion of human HLA-A protein, and a portion of mouse H2-D1 protein.

FIG. 2B shows a schematic diagram of a targeting strategy at mouse B2M gene locus.

FIG. 3A is a schematic diagram showing humanized mouse B2M gene locus. Mouse B2M gene coding region was replaced with a nucleic acid sequence encoding the signal peptide of human HLA-A, human B2M protein, and human HLA-A protein without signal peptide.

FIG. 3B shows a schematic diagram of a targeting strategy at mouse B2M gene locus.

FIGS. 4A-4B show PCR detection result of F1 generation mice by primers WT-F/WT-R or WT-F/Mut-R, respectively. M is marker. H₂O is water control. WT is wildtype control. PC is positive control. F1-01 to F1-16 are mouse numbers.

FIG. 5 shows Southern Blot analysis result of F1 generation mice by 5′ Probe and 3′ Probe. WT is wildtype control. F1-01, F1-02, F1-03, F1-04, F1-05, F1-06, F1-07, F1-08, F1-17, F1-18, F1-19, and F1-20 are mouse numbers.

FIGS. 6A-6F show serum titer curves of antigen-specific antibodies after three immunizations of MHC-I VH/VL mice with an antigen-MHC complex including antigen Peptide A, Peptide B, Peptide C, Peptide D, Peptide E and Peptide F, respectively. Either HLA heterozygous mice (HLA-A*0201^{H/+} hVH/hVL, HLA-A*0201^{H/+} hVH/hcVL) or HLA homozygous mice (HLA-A*0201^{H/H} hVH/hVL) were used.

FIG. 7 shows KD values of 115 antigen-specific antibodies to the Peptide A-MHC complex.

FIG. 8 shows length distribution of VH CDR3 sequences of 16 Peptide A-specific antibodies.

FIGS. 9A-9B show germline IGHV and IGKV gene utilization results for 16 Peptide A-specific antibodies.

FIG. 10 shows length distribution of VH CDR3 sequences of 19 Peptide B-specific antibodies.

FIG. 11A-11B show germline IGHV and IGKV gene utilization results for 19 Peptide B-specific antibodies.

FIG. 12 shows length distribution of VH CDR3 sequences of 18 Peptide C-specific antibodies.

FIG. 13A-13B show germline IGHV and IGKV gene utilization results for 18 Peptide C-specific antibodies.

FIG. 14 is a schematic diagram showing human immunoglobulin heavy chain (IGH) locus on chromosome 14 (14q32.33).

FIG. 15 is a schematic diagram showing mouse (Mus musculus) IGH locus on chromosome 12 (12F2) (strain C57BL/6).

FIG. 16 is a schematic diagram showing human immunoglobulin kappa chain (IGK) locus on chromosome 2 (2p11.2).

FIG. 17 is a schematic diagram showing mouse (Mus musculus) IGK locus on chromosome 6 (6C1).

FIG. 18 shows a serum titer curve of antigen-specific antibodies after immunizing HLA-A*0201 hVH/hcVL heterozygous mice (HLA-A*0201^{H/+} hVH/hcVL v2) with a Peptide F-MHC complex.

FIG. 19 shows the KD values of 30 antigen-specific antibodies to the Peptide F-MHC complex generated by immunizing HLA-A*0201^{H/+} hVH/hcVL v2 mice.

FIG. 20 shows germline IGHV gene utilization results for 29 Peptide-F-specific antibodies generated by immunizing HLA-A*0201^{H/+} hVH/hcVL v2 mice.

FIG. 21 shows serum titer curves of antigen-specific antibodies after immunizing HLA-A*0302 hVH/hVL heterozygous mice (HLA-A*0302^{H/+} hVH/hVL) and HLA-A*0302 hVH/hVL homozygous mice (HLA-A*0302^{H/H} hVH/hVL) with a Peptide G-MHC complex.

FIG. 22 shows serum titer curves of antigen-specific antibodies after immunizing HLA-A*1101 hVH/hVL heterozygous mice (HLA-A*1101^{H/+} hVH/hVL) and HLA-A*1101 hVH/hVL homozygous mice (HLA-A*1101^{H/H} hVH/hVL) with a Peptide G-MHC complex.

FIG. 23 shows the KD values of 24 antigen-specific antibodies after immunizing HLA-A*1101 hVH/hVL mice.

FIG. 24 shows a serum titer curve of antigen-specific antibodies after immunizing HLA-A*2402 hVH/hVL heterozygous mice (HLA-A*2402^{H/+} hVH/hVL) with a Peptide H-MHC complex.

FIG. 25 lists sequences discussed in the disclosure.

### DETAILED DESCRIPTION

The present disclosure relates to genetically modified animals and cells with humanized heavy chain immunoglobulin locus and/or humanized light chain immunoglobulin locus (e.g., kappa chain locus). In some embodiments, the animals and cells can also express a human or chimeric (e.g., humanized) major histocompatibility complex (MHC) protein complex.

The genetically modified animals can express humanized antibodies or chimeric antibodies. For examples, in some cases, the genetically modified animals described herein have complete human antibody repertoires. In some cases, the humanized light chain immunoglobulin locus has a limited set of IGKV genes and IGKJ genes. Because the endogenous IGHV, IGHD, IGHJ, IGKV and IGKJ genes have been effectively deleted, it is less likely that the antibodies generated by the antibody repertoires are immunogenic in humans. Thus, the antibodies are more suitable for being used as therapeutics in humans. Therefore, the genetically modified animals provide an advantageous platform to produce humanized antibodies. Detailed descriptions of RenMab™ mice and RenLite™ mice can be found, e.g., in PCT/CN2020/075698 and PCT/CN2021/097652, respectively, each of which is incorporated herein by reference in its entirety.

The animals and cells can also express a human or chimeric (e.g., humanized) major histocompatibility complex (MHC) protein complex. It has been well established that T cells recognize antigen in association with self MHC proteins but not in association with foreign MHC proteins: that is, T cells show MHC restriction. This restriction results from a process of positive selection during T cell development in the thymus. In this process, those immature T cells that will be capable of recognizing foreign peptides presented by self MHC proteins are selected to survive, while the remainder, which would be of no use to the animal, undergo apoptosis. Thus, MHC restriction is an acquired property of the immune system that emerges as T cells develop in the thymus.

Here, nucleotide sequences encoding a human or chimeric MHC protein complex were introduced into RenMab™ mice and RenLiter™ mice, to obtain genetically-modified non-human animals (MHC-I VH/VL background) that can express and tolerate human MHC protein molecules in vivo. Such animals can be immunized by antigen peptide-MHC complexes to generate TCR-like antibodies with high diversity and high affinity. The VH and VL sequences of the generated antibodies can also be used to prepare chimeric antigen receptors (CARs) or T cell receptors that can target specific cancer antigens.

As used herein, the term “antibody” refers to an immunoglobulin molecule comprising four polypeptide chains, two heavy (H) chains and two light (L) chains inter-connected by disulfide bonds. Each heavy chain comprises a heavy chain variable (VH) domain and a heavy chain constant region (CH). Each light chain comprises a light chain variable (VL) domain and a light chain constant region (CL). The VH and VL domains can be further subdivided into regions of hypervariability, termed complementarity determining regions (CDR), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3). The term “high affinity” antibody refers to an antibody that has a K_{D} with respect to its target epitope about of 10⁻⁹M or lower (e.g., about or lower than 1×10⁻⁹ M, 1×10⁻¹⁰ M, 1×10⁻¹¹ M, or 1×10⁻¹² M). In some embodiments, K_{D} can be measured by surface plasmon resonance, e.g., Biacore™ or ELISA.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by a nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a mouse) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a human antibody and the constant domains of a non-human antibody. Additional examples of chimeric antibodies are described herein and are known in the art.

As used herein, the term “humanized antibody” refers to a non-human antibody which contains sequence derived from a non-human (e.g., mouse) immunoglobulin and contains sequences derived from a human immunoglobulin.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen.

As used herein, the terms “subject” and “patient” are used interchangeably throughout the specification and describe an animal, human or non-human. Veterinary and non-veterinary applications are contemplated by the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

As used herein, the term “an unmodified human sequence” refers to a sequence that is derived from a human subject, a human cell, a cultured human cell or a human cell line, wherein the sequence is identical to the genetic sequence of a human subject, a human cell, a cultured human cell or a human cell line.

### Genetically Modified Heavy Chain Immunoglobulin Locus

Heavy chain immunoglobulin locus (also known as IGH or immunoglobulin heavy locus) is a region on the chromosome (e.g., human chromosome 14) that contains genes for the heavy chains of human antibodies (or immunoglobulins).

This region represents the germline organization of the heavy chain locus. The locus includes V (variable), D (diversity), J (joining), and C (constant) segments. The genes in the V region form a V gene cluster (also known as IGHV gene cluster). The genes in the D region form a D gene cluster (also known as IGHD gene cluster). The genes in the J region form a J gene cluster (also known as IGHJ gene cluster).

During B cell development, a recombination event at the DNA level joins a single D segment (also known as an IGHD gene) with a J segment (also known as an IGHJ gene); the fused D-J exon of this partially rearranged D-J region is then joined to a V segment (also known as an IGHV gene). The rearranged V-D-J region containing a fused V-D-J exon is then transcribed and fused at the RNA level to the IGHM constant region; this transcript encodes a mu heavy chain. Later in development B cells generate V-D-J-Cmu-Cdelta pre-messenger RNA, which is alternatively spliced to encode either a mu or a delta heavy chain. Mature B cells in the lymph nodes undergo switch recombination, so that the fused V-D-J gene segment is brought in proximity to one of the IGHG, IGHA, or IGHE gene segments and each cell expresses either the gamma, alpha, or epsilon heavy chain. Potential recombination of many different IGHV genes with several IGHJ genes provides a wide range of antigen recognition. Additional diversity is attained by junctional diversity, resulting from the random addition of nucleotides by terminal deoxynucleotidyl transferase, and by somatic hypermutation, which occurs during B cell maturation in the spleen and lymph nodes. Several V, D, J, and C segments are known to be incapable of encoding a protein and are considered pseudogenous gene segments (often simply referred to as pseudogenes).

The human heavy chain immunoglobulin locus is located on human chromosome 14. Table 1 lists IGHV genes and its relative orders in this locus.

TABLE 1
List of IGHV genes on human chromosome 14

| Gene names | Order |
|---|---|
| IGHV(III)-82 | 1 |
| IGHV7-81 | 2 |
| IGHV4-80 | 3 |
| IGHV3-79 | 4 |
| IGHV(II)-78-1 | 5 |
| IGHV5-78 | 6 |
| IGHV7-77 | 7 |
| IGHV(III)-76-1 | 8 |
| IGHV3-76 | 9 |
| IGHV3-75 | 10 |
| IGHV(II)-74-1 | 11 |
| IGHV3-74 | 12 |
| IGHV3-73 | 13 |
| IGHV3-72 | 14 |
| IGHV3-71 | 15 |
| IGHV2-70 | 16 |
| IGHV1-69D | 17 |
| IGHV1-69-2 | 18 |
| IGHV3-69-1 | 19 |
| IGHV2-70D | 20 |
| IGHV1-69 | 21 |
| IGHV1-68 | 22 |
| IGHV(III)-67-4 | 23 |
| IGHV(III)-67-3 | 24 |
| IGHV(III)-67-2 | 25 |
| IGHV(II)-67-1 | 26 |
| SLC20A1P1 (GLVR1) | 27 |
| IGHV1-67 | 28 |
| IGHV3-66 | 29 |
| IGHV(II)-65-1 | 30 |
| IGHV3-65 | 31 |
| IGHV3-64 | 32 |
| GOLGA4P3 (Golgin) | 33 |
| IGHV3-63 | 34 |
| IGHV(II)-62-1 | 35 |
| IGHV3-62 | 36 |
| IGHV4-61 | 37 |
| IGHV(II)-60-1 | 38 |
| IGHV3-60 | 39 |
| IGHV4-59 | 40 |
| IGHV1-58 | 41 |
| IGHV3-57 | 42 |
| IGHV7-56 | 43 |
| IGHV4-55 | 44 |
| IGHV3-54 | 45 |
| IGHV(II)-53-1 | 46 |
| IGHV3-53 | 47 |
| IGHV3-52 | 48 |
| IGHV(II)-51-2 | 49 |
| IGHV(III)-51-1 | 50 |
| IGHV5-51 | 51 |
| IGHV3-50 | 52 |
| IGHV(II)-49-1 | 53 |
| IGHV3-49 | 54 |
| IGHV3-48 | 55 |
| IGHV(III)-47-1 | 56 |
| IGHV3-47 | 57 |
| IGHV(II)-46-1 | 58 |
| IGHV1-46 | 59 |
| IGHV1-45 | 60 |
| IGHV(II)-44-2 | 61 |
| IGHV(IV)-44-1 | 62 |
| IGHV(III)-44 | 63 |
| IGHV(II)-43-1 | 64 |
| IGHV3-43 | 65 |
| IGHV3-42 | 66 |
| IGHV3-41 | 67 |
| IGHV(II)-40-1 | 68 |
| IGHV7-40 | 69 |
| IGHV4-39 | 70 |
| IGHV1-38-4 | 71 |
| IGHV(III)-38-1D | 72 |
| IGHV3-38-3 | 73 |
| IGHV(III)-44D | 74 |
| IGHV(II)-43-1D | 75 |
| IGHV3-43D | 76 |
| IGHV3-42D | 77 |
| IGHV7-40D | 78 |
| IGHV4-38-2 | 79 |
| IGHV(III)-38-1 | 80 |
| IGHV3-38 | 81 |
| IGHV3-37 | 82 |
| IGHV3-36 | 83 |
| IGHV3-35 | 84 |
| IGHV7-34-1 | 85 |
| IGHV4-34 | 86 |
| IGHV3-33-2 | 87 |
| IGHV(II)-33-1 | 88 |
| IGHV3-33 | 89 |
| GOLGA4P1 (Golgin) | 90 |
| IGHV3-32 | 91 |
| IGHV(II)-31-1 | 92 |
| IGHV4-31 | 93 |
| IGHV3-30-52 | 94 |
| IGHV(II)-30-51 | 95 |
| IGHV3-30-5 | 96 |
| IGHV3-30-42 | 97 |
| IGHV(II)-30-41 | 98 |
| IGHV4-30-4 | 99 |
| IGHV3-30-33 | 100 |
| IGHV(II)-30-32 | 101 |
| IGHV3-30-3 | 102 |
| IGHV3-30-22 | 103 |
| IGHV(II)-30-21 | 104 |
| IGHV4-30-2 | 105 |
| IGHV4-30-1 | 106 |
| IGHV3-30-2 | 107 |
| IGHV(II)-30-1 | 108 |
| IGHV3-30 | 109 |
| GOLGA4P2 (Golgin) | 110 |
| IGHV3-29 | 111 |
| IGHV(II)-28-1 | 112 |
| IGHV4-28 | 113 |
| IGHV7-27 | 114 |
| IGHV(II)-26-2 | 115 |
| IGHV(III)-26-1 | 116 |
| IGHV2-26 | 117 |
| IGHV(III)-25-1 | 118 |
| IGHV3-25 | 119 |
| IGHV1-24 | 120 |
| IGHV3-23D | 121 |
| IGHV(III)-22-2D | 122 |
| IGHV(II)-22-1D | 123 |
| IGHV3-23 | 124 |
| IGHV(III)-22-2 | 125 |
| IGHV(II)-22-1 | 126 |
| IGHV3-22 | 127 |
| IGHV3-21 | 128 |
| IGHV(II)-20-1 | 129 |
| IGHV3-20 | 130 |
| IGHV3-19 | 131 |
| IGHV1-18 | 132 |
| SLC20A1P2 | 133 |
| IGHV1-17 | 134 |
| IGHV(III)-16-1 | 135 |
| IGHV3-16 | 136 |
| IGHV(II)-15-1 | 137 |
| IGHV3-15 | 138 |
| IGHV1-14 | 139 |
| IGHV(III)-13-1 | 140 |
| IGHV3-13 | 141 |
| IGHV1-12 | 142 |
| IGHV(III)-11-1 | 143 |
| IGHV3-11 | 144 |
| IGHV2-10 | 145 |
| IGHV3-9 | 146 |
| IGHV1-8 | 147 |
| IGHV5-10-1 | 148 |
| IGHV3-64D | 149 |
| IGHV3-7 | 150 |
| IGHV3-6 | 151 |
| IGHV(III)-5-2 | 152 |
| IGHV(III)-5-1 | 153 |
| IGHV2-5 | 154 |
| IGHV7-4-1 | 155 |
| IGHV4-4 | 156 |
| IGHV1-3 | 157 |
| IGHV(III)-2-1 | 158 |
| IGHV1-2 | 159 |
| | * |
| | * |
| IGHV(II)-1-1 | 162 |
| IGHV6-1 | 163 |
| | * |

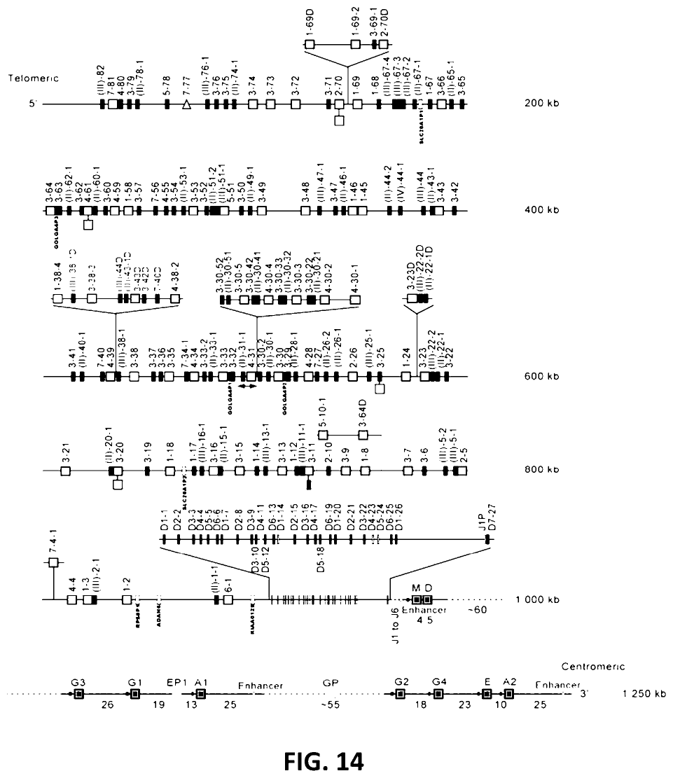

RPS8P1, ADAM6, and KIAA0125 are also located in this locus. The relative order of RPS8P 1 is 160, the relative order of ADAM6 is161, and the relative order of KIAA0125 is 164. Table 2 lists all IGHD genes and its relative orders on human chromosome 14. Table 3 lists all IGHJ genes and its relative orders on human chromosome 14. The genes for immunoglobulin constant domains are located after the IGHV, IGHD, and IGHJ genes. These genes include (as shown in the following order): immunoglobulin heavy constant mu (IGHM), immunoglobulin heavy constant delta (IGH δ), immunoglobulin heavy constant gamma 3 (IGHG3), immunoglobulin heavy constant gamma 1 (IGHG1), immunoglobulin heavy constant epsilon β1 (pseudogene) (IGHEP1), immunoglobulin heavy constant alpha 1 (IGHA1), immunoglobulin heavy constant gamma P (non-functional) (IGHGP), immunoglobulin heavy constant gamma 2 (IGHG2), immunoglobulin heavy constant gamma 4 (IGHG4), immunoglobulin heavy constant epsilon (IGHE), and immunoglobulin heavy constant alpha 2 (IGHA2). These genes and the order of these genes are also shown in FIG. 14.

TABLE 2
List of IGHD genes on human chromosome 14

| Gene names | Order |
|---|---|
| IGHD1-1 | 165 |
| IGHD2-2 | 166 |
| IGHD3-3 | 167 |
| IGHD4-4 | 168 |
| IGHD5-5 | 169 |
| IGHD6-6 | 170 |
| IGHD1-7 | 171 |
| IGHD2-8 | 172 |
| IGHD3-9 | 173 |
| IGHD3-10 | 174 |
| IGHD4-11 | 175 |
| IGHD5-12 | 176 |
| IGHD6-13 | 177 |
| IGHD1-14 | 178 |
| IGHD2-15 | 179 |
| IGHD3-16 | 180 |
| IGHD4-17 | 181 |
| IGHD5-18 | 182 |
| IGHD6-19 | 183 |
| IGHD1-20 | 184 |
| IGHD2-21 | 185 |
| IGHD3-22 | 186 |
| IGHD4-23 | 187 |
| IGHD5-24 | 188 |
| IGHD6-25 | 189 |
| IGHD1-26 | 190 |
| | * |
| IGHD7-27 | 192 |

TABLE 3
List of IGHJ genes on human chromosome 14

| Gene names | Order |
|---|---|
| IGHJ1P | 191 |
| IGHJ1 | 193 |
| IGHJ2 | 194 |
| IGHJ2P | 195 |
| IGHJ3 | 196 |
| IGHJ4 | 197 |
| IGHJ5 | 198 |
| IGHJ3P | 199 |
| IGHJ6 | 200 |

The mouse heavy chain immunoglobulin locus is located on mouse chromosome 12. Table 4 lists IGHV genes and its relative orders in this locus.

TABLE 4
List of IGHV genes on mouse chromosome 12

| Gene names | Order |
|---|---|
| IGHV1-86 | 1 |
| IGHV1-85 | 2 |
| IGHV1-84 | 3 |
| IGHV1-83 | 4 |
| IGHV1-82 | 5 |
| IGHV1-81 | 6 |
| IGHV1-80 | 7 |
| IGHV1-79 | 8 |
| IGHV1-78 | 9 |
| IGHV1-77 | 10 |
| IGHV8-16 | 11 |
| IGHV1-76 | 12 |
| IGHV8-15 | 13 |
| IGHV1-75 | 14 |
| IGHV8-14 | 15 |
| IGHV1-74 | 16 |
| IGHV1-73 | 17 |
| IGHV8-13 | 18 |
| IGHV1-72 | 19 |
| IGHV1-71 | 20 |
| IGHV1-70 | 21 |
| IGHV8-12 | 22 |
| IGHV1-69 | 23 |
| IGHV1-68 | 24 |
| IGHV1-67 | 25 |
| IGHV1-66 | 26 |
| IGHV8-11 | 27 |
| IGHV1-65 | 28 |
| IGHV8-10 | 29 |
| IGHV1-64 | 30 |
| IGHV1-63 | 31 |
| IGHV8-9 | 32 |
| IGHV1-62-3 | 33 |
| IGHV1-62-2 | 34 |
| IGHV1-62-1 | 35 |
| IGHV1-62 | 36 |
| IGHV1-61 | 37 |
| IGHV1-60 | 38 |
| IGHV1-59 | 39 |
| IGHV1-58 | 40 |
| IGHV8-8 | 41 |
| IGHV1-57 | 42 |
| IGHV8-7 | 43 |
| IGHV1-56 | 44 |
| IGHV1-55 | 45 |
| IGHV1-54 | 46 |
| IGHV8-6 | 47 |
| IGHV1-53 | 48 |
| IGHV1-52 | 49 |
| IGHV1-51 | 50 |
| IGHV1-50 | 51 |
| IGHV8-5 | 52 |
| IGHV1-49 | 53 |
| IGHV1-48 | 54 |
| IGHV8-4 | 55 |
| IGHV8-3 | 56 |
| IGHV1-47 | 57 |
| IGHV1-46 | 58 |
| IGHV1-45 | 59 |
| IGHV1-44 | 60 |
| IGHV1-43 | 61 |
| IGHV1-42 | 62 |
| IGHV1-41 | 63 |
| IGHV1-40 | 64 |
| IGHV1-39 | 65 |
| IGHV1-38 | 66 |
| IGHV1-37 | 67 |
| IGHV1-36 | 68 |
| IGHV1-35 | 69 |
| IGHV1-34 | 70 |
| IGHV1-33 | 71 |
| IGHV1-32 | 72 |
| IGHV1-31 | 73 |
| IGHV1-30 | 74 |
| IGHV1-29 | 75 |
| IGHV1-28 | 76 |
| IGHV1-27 | 77 |
| IGHV1-26 | 78 |
| IGHV1-25 | 79 |
| IGHV1-24 | 80 |
| IGHV1-23 | 81 |
| IGHV1-22 | 82 |
| IGHV1-21 | 83 |
| IGHV1-21-1 | 84 |
| IGHV1-20 | 85 |
| IGHV1-19 | 86 |
| IGHV1-19-1 | 87 |
| IGHV1-18 | 88 |
| IGHV1-17 | 89 |
| IGHV1-17-1 | 90 |
| IGHV1-16 | 91 |
| IGHV1-15 | 92 |
| IGHV1-14 | 93 |
| IGHV1-13 | 94 |
| IGHV1-12 | 95 |
| IGHV1-11 | 96 |
| IGHV1-10 | 97 |
| IGHV1-9 | 98 |
| IGHV15-2 | 99 |
| IGHV1-8 | 100 |
| IGHV10-4 | 101 |
| IGHV1-7 | 102 |
| IGHV1-6 | 103 |
| IGHV10-3 | 104 |
| IGHV1-5 | 105 |
| IGHV10-2 | 106 |
| IGHV1-4 | 107 |
| IGHV1-3 | 108 |
| IGHV10-1 | 109 |
| IGHV1-2 | 110 |
| IGHV8-2 | 111 |
| IGHV6-7 | 112 |
| IGHV6-6 | 113 |
| IGHV6-5 | 114 |
| IGHV6-4 | 115 |
| IGHV6-3 | 116 |
| IGHV12-3 | 117 |
| IGHV13-2 | 118 |
| IGHV1-1 | 119 |
| IGHV8-1 | 120 |
| IGHV3-8 | 121 |
| IGHV5-21 | 122 |
| IGHV3-7 | 123 |
| IGHV9-4 | 124 |
| IGHV3-6 | 125 |
| IGHV13-1 | 126 |
| IGHV3-5 | 127 |
| IGHV3-4 | 128 |
| IGHV7-4 | 129 |
| IGHV3-3 | 130 |
| IGHV14-4 | 131 |
| IGHV15-1 | 132 |
| IGHV7-3 | 133 |
| IGHV9-3 | 134 |
| IGHV12-2 | 135 |
| IGHV9-2 | 136 |
| IGHV12-1 | 137 |
| IGHV9-1 | 138 |
| IGHV6-2 | 139 |
| IGHV16-1 | 140 |
| IGHV14-3 | 141 |
| IGHV11-2 | 142 |
| IGHV3-2 | 143 |
| IGHV4-2 | 144 |
| IGHV14-2 | 145 |
| IGHV11-1 | 146 |
| IGHV3-1 | 147 |
| IGHV4-1 | 148 |
| IGHV14-1 | 149 |
| IGHV7-2 | 150 |
| IGHV7-1 | 151 |
| IGHV5-19 | 152 |
| IGHV2-9 | 153 |
| IGHV2-8 | 154 |
| IGHV5-18 | 155 |
| IGHV5-17 | 156 |
| IGHV5-16 | 157 |
| IGHV5-15 | 158 |
| IGHV2-7 | 159 |
| IGHV2-6-8 | 160 |
| IGHV2-9-1 | 161 |
| IGHV5-12-4 | 162 |
| IGHV5-9-1 | 163 |
| IGHV2-6 | 164 |
| IGHV5-12 | 165 |
| IGHV5-11 | 166 |
| IGHV2-5 | 167 |
| IGHV5-10 | 168 |
| IGHV5-9 | 169 |
| IGHV5-8 | 170 |
| IGHV2-4 | 171 |
| IGHV5-7 | 172 |
| IGHV5-6 | 173 |
| IGHV5-5 | 174 |
| IGHV2-3 | 175 |
| IGHV6-1 | 176 |
| IGHV5-4 | 177 |
| IGHV5-3 | 178 |
| IGHV2-2 | 179 |
| IGHV5-2 | 180 |
| IGHV2-1 | 181 |
| IGHV5-1 | 182 |

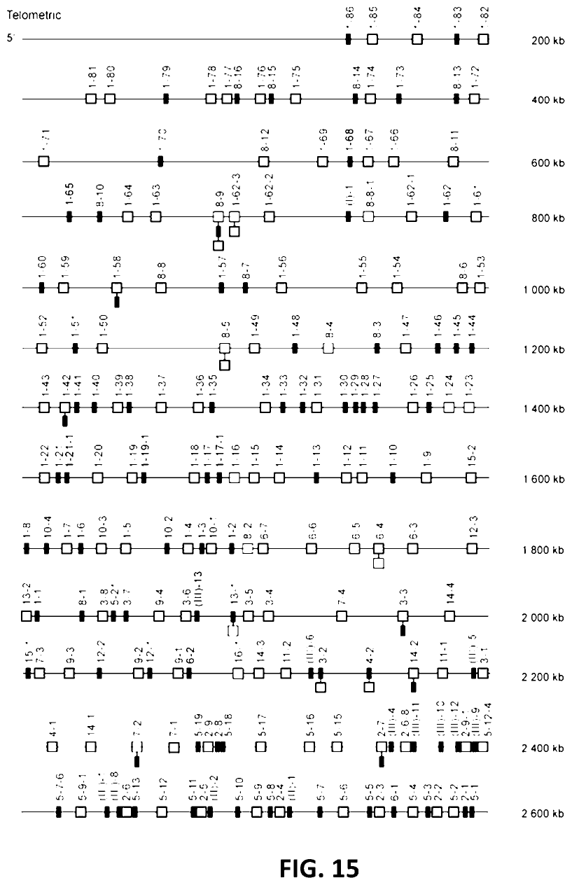

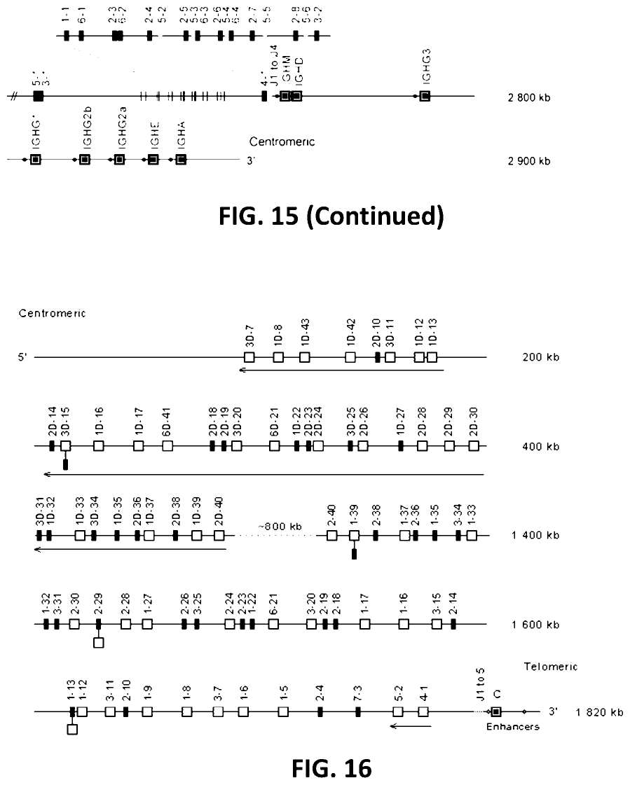

Table 5 lists all IGHD genes and its relative orders on mouse chromosome 12. Table 6 lists all IGHJ genes and its relative orders on mouse chromosome 12. The genes for immunoglobulin constant domains are after the IGHV, IGHD, and IGHJ genes. These genes include (as shown in the following order): immunoglobulin heavy constant mu (IGHM), immunoglobulin heavy constant delta (IGH δ), immunoglobulin heavy constant gamma 3 (IGHG3), immunoglobulin heavy constant gamma 1 (IGHG1). immunoglobulin heavy constant gamma 2b (IGHG2b), immunoglobulin heavy constant gamma 2a (IGHG2a), immunoglobulin heavy constant epsilon (IGHE), and immunoglobulin heavy constant alpha (IGHA) genes. These genes and the order of these genes are also shown in FIG. 15.

TABLE 5
List of IGHD genes on mouse chromosome 12

| Gene names | Order |
|---|---|
| IGHD5-1 | 183 |
| IGHD3-1 | 184 |
| IGHD1-1 | 185 |
| IGHD6-1 | 186 |
| IGHD2-3 | 187 |
| IGHD6-2 | 188 |
| IGHD2-4 | 189 |
| IGHD2-5 | 191 |
| IGHD5-3 | 192 |
| IGHD5-7 | 193 |
| IGHD2-6 | 194 |
| IGHD5-4 | 195 |
| IGHD5-8 | 196 |
| IGHD2-7 | 197 |
| IGHD5-5 | 198 |
| IGHD2-8 | 199 |
| IGHD5-6 | 200 |
| IGHD3-2 | 201 |
| IGHD4-1 | 202 |

TABLE 6
List of IGHJ genes on mouse chromosome 12

| Gene names | Order |
|---|---|
| IGHJ1 | 203 |
| IGHJ2 | 204 |
| IGHJ3 | 205 |
| IGHJ4 | 206 |

The present disclosure provides genetically-modified, non-human animal comprising one or more human IGHV genes, one or more human IGHD genes, and/or one or more human IGHJ genes. In some embodiments, the human IGHV genes, the human IGHD genes, and the human IGHJ genes are operably linked together and can undergo VDJ rearrangement. In some embodiments, the human IGHV genes, the human IGHD genes, and the human IGHJ genes are at the endogenous heavy chain immunoglobulin gene locus.

In some embodiments, the animal comprises about or at least 1, 2, 3, 4, 5, 6, 7, 8, 9, 10, 20, 30, 40, 50, 60, 70, 80, 90, 100, 110, 120, 130, 140, 150, 151, 152, 153, 154, 155, 156, 157, 158, 159, 160 or 161 human IGHV genes (e.g., genes as shown in Table 1).

In some embodiments, the animal comprises 1, 2, 3, 4, 5, 6, 7, 8, 9, or 10 genes selected from IGHV(III)-82, IGHV7-81, IGHV4-80, IGHV3-79, IGHV(II)-78-1, IGHV5-78, IGHV7-77, IGHV(III)-76-1, IGHV3-76, and IGHV3-75.

In some embodiments, the animal comprises 1, 2, 3, 4, 5, 6, 7, 8, 9, or 10 genes selected from IGHV(III)-5-2, IGHV(III)-5-1, IGHV2-5, IGHV7-4-1, IGHV4-4, IGHV1-3, IGHV(III)-2-1, IGHV1-2, IGHV(II)-1-1, and IGHV6-1.

In some embodiments, the animal comprises an unmodified human sequence comprising a sequence starting from a gene selected from IGHV(III)-82, IGHV7-81, IGHV4-80, IGHV3-79, IGHV(II)-78-1, IGHV5-78, IGHV7-77, IGHV(III)-76-1, IGHV3-76, and IGHV3-75, and ending at a gene selected from IGHV(III)-5-2, IGHV(III)-5-1, IGHV2-5, IGHV7-4-1, IGHV4-4, IGHV1-3, IGHV(III)-2-1, IGHV1-2, IGHV(II)-1-1, and IGHV6-1. In some embodiments, the unmodified human sequence derived from a human heavy chain immunoglobulin gene locus starting from human IGHV(III)-82 to human IGHV1-2. In some embodiments, the unmodified human sequence derived from a human heavy chain immunoglobulin gene locus starting from human IGHV(III)-82 to human IGHV(II)-1-1. In some embodiments, the unmodified human sequence derived from a human heavy chain immunoglobulin gene locus starting from human IGHV(III)-82 to human IGHV-6-1.

In some embodiments, the animal comprises about or at least 1, 2, 3, 4, 5, 6, 7, 8, 9, 10, 11, 12, 13, 14, 15, 16, 17, 18, 19, 20, 21, 22, 23, 24, 25, 26, or 27 human IGHD genes (e.g., genes as shown in Table 2). In some embodiments, the animal comprises 1, 2, 3, 4, 5, 6, 7, 8, 9, or 10 genes selected from IGHD1-1, IGHD2-2, IGHD3-3, IGHD4-4, IGHD5-5, IGHD4-23, IGHD5-24, IGHD6-25, IGHD1-26, and IGHD7-27.

In some embodiments, the animal comprises about or at least 1, 2, 3, 4, 5, 6, 7, 8, or 9 human IGHJ genes (e.g., genes as shown in Table 3). In some embodiments, the animal comprises 1, 2, 3, 4, 5, 6, 7, 8, or 9 human IGHJ genes selected from IGHJ1P, IGHJ1, IGHJ2, IGHJ2P, IGHJ3, IGHJ4, IGHJ5, IGHJ3P, and IGHJ6.

In some embodiments, the animal comprises an unmodified human sequence comprising a sequence starting from a gene selected from IGHD1-1, IGHD2-2, IGHD3-3, IGHD4-4, IGHD5-5, IGHD4-23, IGHD5-24, IGHD6-25, IGHD1-26, and IGHD7-27, and ending at a gene selected from IGHJ1P, IGHJ1, IGHJ2, IGHJ2P, IGHJ3, IGHJ4, IGHJ5, IGHJ3P, and IGHJ6. In some embodiments, the unmodified human sequence derived from a human heavy chain immunoglobulin gene locus starting from human IGHD1-1 to human IGHJ6.

In some embodiments, the unmodified human sequence derived from a human heavy chain immunoglobulin gene locus starting from human IGHD1-1 to human IGHD7-27.

In some embodiments, the unmodified human sequence derived from a human heavy chain immunoglobulin gene locus starting from human IGHJ1P to human IGHJ6. In some embodiments, the unmodified human sequence derived from a human heavy chain immunoglobulin gene locus starting from human IGHJ1 to human IGHJ6.

In some embodiments, the unmodified human sequence derived from a human heavy chain immunoglobulin gene locus starting from human IGHV(III)-82 to human IGHJ6.

In some embodiments, the unmodified human sequence derived from a human heavy chain immunoglobulin gene locus starting from human IGHV1-2 to human IGHJ6. In some embodiments, the unmodified human sequence derived from a human heavy chain immunoglobulin gene locus starting from human IGHV(II)-1-1 to human IGHJ6. In some embodiments, the unmodified human sequence derived from a human heavy chain immunoglobulin gene locus starting from human IGHV6-1 to human IGHJ6.

In some embodiments, the animal can have one, two, three, four, five, six, seven, eight, nine, or ten unmodified human sequences. In some embodiments, the unmodified human sequence has a length of about or at least 10, 20, 30, 40, 50, 60, 70, 80, 90, 100, 200, 300, 400, 500, 600, 700, 800, 900, or 1000 kb.

In some embodiments, the animal comprises one or more endogenous genes selected from the group consisting of immunoglobulin heavy constant mu (IGHM), immunoglobulin heavy constant delta (IGHδ), immunoglobulin heavy constant gamma 3 (IGHG3), immunoglobulin heavy constant gamma 1 (IGHG1), immunoglobulin heavy constant gamma 2b (IGHG2b), immunoglobulin heavy constant gamma 2a (IGHG2a), immunoglobulin heavy constant epsilon (IGHE), and immunoglobulin heavy constant alpha (IGHA) genes. In some embodiments, these endogenous genes are operably linked together. In some embodiments, these endogenous genes have the same order as in a wildtype animal. In some embodiments, isotype switching (immunoglobulin class switching) can occur in the animal.

In some embodiments, the IGHV genes, the IGHD genes, and/or the IGHJ genes are operably linked together. The VDJ recombination can occur among these genes and produce functional antibodies. In some embodiments, these genes are arranged in an order that is similar to the order in human heavy chain immunoglobulin locus. This arrangement offers various advantages, e.g., the arrangement of these genes allow the production of heavy chain variable domains with a diversity that is very similar to the diversity of the heavy chain variable domains in human. As some random sequences may be inserted to the sequence during VDJ recombination, in some embodiments, the complete human antibody repertoires with no or minimum modifications can reduce the likelihood that non-human sequence is inserted during the VDJ recombination.

In some embodiments, the IGHV genes, the IGHD genes, and/or the IGHJ genes are operably linked together to one or more genes (e.g., all genes) selected from IGHM, IGHδ, IGHG3, IGHG1, IGHG2b, IGHG2a, IGHE, and IGHA genes.

In some embodiments, the animal comprises a disruption in the animal's endogenous heavy chain immunoglobulin gene locus. In some embodiments, the disruption in the animal's endogenous heavy chain immunoglobulin gene locus comprises a deletion of one or more endogenous IGHV genes, one or more endogenous IGHD genes, and one or more endogenous IGHJ genes.

In some embodiments, the animal is a mouse. The disruption in the animal's endogenous heavy chain immunoglobulin gene locus comprises a deletion of at least or about 1, 2, 3, 4, 5, 6, 7, 8, 9, 10, 20, 30, 40, 50, 60, 70, 80, 90, 100, 110, 120, 130, 140, 150, 160, 170, 171, 172, 173, 174, 175, 176, 177, 178, 179, 180, 181, or 182 mouse IGHV genes (e.g., genes as shown in Table 4). In some embodiments, the disruption comprises a deletion of about or at least 1, 2, 3, 4, 5, 6, 7, 8, 9, or 10 mouse IGHV genes selected from IGHV1-86, IGHV1-85, IGHV1-84, IGHV1-83, IGHV1-82, IGHV1-81, IGHV1-80, IGHV1-79, IGHV1-78, and IGHV1-77. In some embodiments, the mouse still comprises about or at least 1, 2, 3, 4, 5, 6, 7, 8, 9, or 10 mouse IGHV genes selected from IGHV1-86, IGHV1-85, IGHV1-84, IGHV1-83, IGHV1-82, IGHV1-81, IGHV1-80, IGHV1-79, IGHV1-78, and IGHV1-77 (e.g., IGHV1-86).

In some embodiments, the disruption comprises a deletion of about or at least 1, 2, 3, 4, 5, 6, 7, 8, 9, or 10 mouse IGHV genes selected from IGHV5-6, IGHV5-5, IGHV2-3, IGHV6-1, IGHV5-4, IGHV5-3, IGHV2-2, IGHV5-2, IGHV2-1, and IGHV5-1. In some embodiments, the mouse still comprises a deletion of about or at least 1, 2, 3, 4, 5, 6, 7, 8, 9, or 10 mouse IGHV genes selected from IGHV5-6, IGHV5-5, IGHV2-3, IGHV6-1, IGHV5-4, IGHV5-3, IGHV2-2, IGHV5-2, IGHV2-1, and IGHV5-1.

In some embodiments, the disruption in the animal's endogenous heavy chain immunoglobulin gene locus comprises a deletion of at least or about 1, 2, 3, 4, 5, 6, 7, 8, 9, 10, 11, 12, 13, 14, 15, 16, 17, 18, 19, or 20 mouse IGHD genes (e.g., genes as shown in Table 5). In some embodiments, the disruption comprises a deletion of about or at least 1, 2, 3, 4, 5, 6, 7, 8, 9, or 10 mouse IGHD genes selected from IGHD5-1, IGHD3-1, IGHD1-1, IGHD6-1, IGHD2-3, IGHD2-7, IGHD2-8, IGHD5-6, IGHD3-2, and IGHD4-1. In some embodiments, the mouse still comprises about or at least 1, 2, 3, 4, 5, 6, 7, 8, 9, or 10 mouse IGHD genes selected from IGHD5-1, IGHD3-1, IGHD1-1, IGHD6-1, IGHD2-3, IGHD2-7, IGHD2-8, IGHD5-6, IGHD3-2, and IGHD4-1.

In some embodiments, the disruption comprises a deletion of about or at least 1, 2, 3, or 4 mouse IGHJ genes selected from IGHJ1, IGHJ2, IGHJ3, and IGHJ4. In some embodiments, the mouse still comprises about or at least 1, 2, 3, or 4 mouse IGHJ genes selected from IGHJ1, IGHJ2, IGHJ3, and IGHJ4.

In some embodiments, the disruption in the animal's endogenous heavy chain immunoglobulin gene locus comprises a deletion of about or at least 500 kb, 600 kb, 700 kb, 800 kb, 900 kb, 1000 kb, 1500 kb, 2000 kb, 2500 kb, or 3000 kb of an endogenous sequence.

In some embodiments, the deleted sequence starts from IGHV1-86 to IGHJ4, from IGHV1-85 to IGHJ4, from IGHV1-84 to IGHJ4, from IGHV1-83 to IGHJ4, or from IGHV1-82 to IGHJ4 (e.g., from IGHV1-85 to IGHJ4).

In some embodiments, the animal comprises about or at least 1, 2, 3, 4, 5, 6, 7, 8, 9, or 10 sequences that are at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence in the human heavy chain immunoglobulin gene locus. In some embodiments, the sequence has a length of about or at least 10, 20, 30, 40, 50, 60, 70, 80, 90, 100, 200, 300, 400, 500, 600, 700, 800, 900, 1000, 1500, 2000, 2500, 3000 or 3500 kb. In some embodiments, the sequence starts from human IGHV(III)-82 to IGHV1-2. In some embodiments, the sequence starts from human IGHV7-81 to IGHV1-2. In some embodiments, the sequence starts from human IGHV(II)-1-1 to IGHVJ6. In some embodiments, the sequence starts from human IGHV6-1 to IGHVJ6.

The human IGHV genes, the human IGHD genes, and the human IGHJ genes are operably linked together and can undergo VDJ rearrangement. In some embodiments, the modified mouse has complete human IGHV, IGHD, and IGHJ gene repertoires (e.g., including all non-pseudo human IGHV, IGHD, and IGHJ genes). Thus, the modified mouse can produce a complete human antibody repertory. In some embodiments, after VDJ recombination, one IGHV gene (e.g., IGHV3-21 or IGHV3-74) contributes to the sequence that encodes an antibody heavy chain variable region. One IGHD gene contributes to the sequence that encodes an antibody heavy chain variable region. And one IGHJ gene contributes to the sequence that encodes an antibody heavy chain variable region. In some embodiments, the IGHV gene is IGHV3-21 or IGHV3-74.

Furthermore, in some cases, the entire mouse IGHV genes, IGHD genes, and IGHJ genes (e.g., including all none-pseudo genes) are knocked out, and the heavy chain variable region will not have any sequence that is encoded by a sequence derived from the mouse, thereby minimizing immunogenicity in human.

### Genetically Modified Kappa Light Chain Immunoglobulin Locus

Kappa chain immunoglobulin locus (also known as IGK or immunoglobulin kappa locus) is a region on the chromosome (e.g., human chromosome 2) that contains genes for the light chains of human antibodies (or immunoglobulins). Similarly, the immunoglobulin light chain genes can also undergo a series rearrangement that lead to the production of a mature immunoglobulin light-chain nucleic acid (e.g., a kappa chain).

The joining of a V segment (also known as an IGKV gene) and a J segment (also known as an IGKJ gene) creates a continuous exon that encodes the whole of the light-chain variable domain. In the unrearranged DNA, the V gene segments (or IGKV gene cluster) are located relatively far away from the C region. The J gene segments (or IGKJ gene cluster) are located close to the C region. Joining of a V segment to a J gene segment also brings the V gene close to a C-region sequence. The J gene segment of the rearranged V region is separated from a C-region sequence only by an intron. To make a complete immunoglobulin light-chain messenger RNA, the V-region exon is joined to the C-region sequence by RNA splicing after transcription.

The human light chain immunoglobulin locus is located on human chromosome 2. Table 7 lists IGKV genes and its relative orders in this locus. There are several different groups for human IGKV genes, including IGKV1 genes (including all IGKV genes starting with IGKV1, also known as VκI), IGKV2 genes (including all IGKV genes starting with IGKV2, also known as VκII), IGKV3 genes (including all IGKV genes starting with IGKV3, also known as VκIII), IGKV4 genes (including all IGKV genes starting with IGKV4, also known as VκIV), IGKV5 genes (including all IGKV genes starting with IGKV5, also known as VκV), IGKV6 genes (including all IGKV genes starting with IGKV6, also known as VκVI), and IGKV7 genes (including all IGKV genes starting with IGKV7, also known as VκVII).

These IGKV genes in human chromosome 2 also form two clusters, the proximal Vκ cluster and the distal Vκ cluster. The sequences in the two clusters are similar but are not identical. This large segmental duplication of the sequence occurred since the divergence of the human lineage from the most recent shared ancestor with other great apes.

TABLE 7
List of IGKV genes on human chromosome 2

| Gene names | Order |
|---|---|
| IGKV3D-7 | 1 |
| IGKV1D-8 | 2 |
| IGKV1D-43 | 3 |
| IGKV1D-42 | 4 |
| IGKV2D-10 | 5 |
| IGKV3D-11 | 6 |
| IGKV1D-12 | 7 |
| IGKV1D-13 | 8 |
| IGKV2D-14 | 9 |
| IGKV3D-15 | 10 |
| IGKV1D-16 | 11 |
| IGKV1D-17 | 12 |
| IGKV6D-41 | 13 |
| IGKV2D-18 | 14 |
| IGKV2D-19 | 15 |
| IGKV3D-20 | 16 |
| IGKV6D-21 | 17 |
| IGKV1D-22 | 18 |
| IGKV2D-23 | 19 |
| IGKV2D-24 | 20 |
| IGKV3D-25 | 21 |
| IGKV2D-26 | 22 |
| IGKV1D-27 | 23 |
| IGKV2D-28 | 24 |
| IGKV2D-29 | 25 |
| IGKV2D-30 | 26 |
| IGKV3D-31 | 27 |
| IGKV1D-32 | 28 |
| IGKV1D-33 | 29 |
| IGKV3D-34 | 30 |
| IGKV1D-35 | 31 |
| IGKV2D-36 | 32 |
| IGKV1D-37 | 33 |
| IGKV2D-38 | 34 |
| IGKV1D-39 | 35 |
| IGKV2D-40 | 36 |
| IGKV2-40 | 37 |
| IGKV1-39 | 38 |
| IGKV2-38 | 39 |
| IGKV1-37 | 40 |
| IGKV2-36 | 41 |
| IGKV1-35 | 42 |
| IGKV3-34 | 43 |
| IGKV1-33 | 44 |
| IGKV1-32 | 45 |
| IGKV3-31 | 46 |
| IGKV2-30 | 47 |
| IGKV2-29 | 48 |
| IGKV2-28 | 49 |
| IGKV1-27 | 50 |
| IGKV2-26 | 51 |
| IGKV3-25 | 52 |
| IGKV2-24 | 53 |
| IGKV2-23 | 54 |
| IGKV1-22 | 55 |
| IGKV6-21 | 56 |
| IGKV3-20 | 57 |
| IGKV2-19 | 58 |
| IGKV2-18 | 59 |
| IGKV1-17 | 60 |
| IGKV1-16 | 61 |
| IGKV3-15 | 62 |
| IGKV2-14 | 63 |
| IGKV1-13 | 64 |
| IGKV1-12 | 65 |
| IGKV3-11 | 66 |
| IGKV2-10 | 67 |
| IGKV1-9 | 68 |
| IGKV1-8 | 69 |
| IGKV3-7 | 70 |
| IGKV1-6 | 71 |
| IGKV1-5 | 72 |
| IGKV2-4 | 73 |
| IGKV7-3 | 74 |
| IGKV5-2 | 75 |
| IGKV4-1 | 76 |

Table 8 lists all IGKJ genes and its relative orders on human chromosome 2. The immunoglobulin kappa constant (IGKC) gene, which encodes the light chain immunoglobulin constant domains is located after the IGKV and IGKJ genes. These genes and the order of these genes are also shown in FIG. 16.

TABLE 8
List of IGKJ genes on human chromosome 2

| Gene names | Order |
|---|---|
| IGKJ1 | 77 |
| IGKJ2 | 78 |
| IGKJ3 | 79 |
| IGKJ4 | 80 |
| IGKJ5 | 81 |

The mouse light chain immunoglobulin locus is located on mouse chromosome 6. Table 9 lists IGKV genes and its relative orders in this locus.

TABLE 9
List of IGKV genes on mouse chromosome 6

| Gene names | Order |
|---|---|
| IGKV2-137 | 1 |
| IGKV1-136 | 2 |
| IGKV1-135 | 3 |
| | * |
| IGKV14-134-1 | 5 |
| IGKV17-134 | 6 |
| IGKV1-133 | 7 |
| IGKV1-132 | 8 |
| IGKV1-131 | 9 |
| IGKV14-130 | 10 |
| IGKV9-129 | 11 |
| IGKV9-128 | 12 |
| IGKV17-127 | 13 |
| IGKV14-126-1 | 14 |
| IGKV14-126 | 15 |
| IGKV11-125 | 16 |
| IGKV9-124 | 17 |
| IGKV9-123 | 18 |
| IGKV1-122 | 19 |
| IGKV17-121 | 20 |
| IGKV9-120 | 21 |
| IGKV9-119 | 22 |
| IGKV14-118-2 | 23 |
| IGKV14-118-1 | 24 |
| IGKV11-118 | 25 |
| IGKV1-117 | 26 |
| IGKV2-116 | 27 |
| IGKV1-115 | 28 |
| IGKV11-114 | 29 |
| IGKV2-113 | 30 |
| IGKV2-112 | 31 |
| IGKV14-111 | 32 |
| IGKV1-110 | 33 |
| IGKV2-109 | 34 |
| IGKV1-108 | 35 |
| IGKV2-107 | 36 |
| IGKV11-106 | 37 |
| IGKV2-105 | 38 |
| IGKV16-104 | 39 |
| IGKV15-103 | 40 |
| IGKV15-102 | 41 |
| IGKV20-101-2 | 42 |
| IGKV15-101-1 | 43 |
| IGKV15-101 | 44 |
| IGKV14-100 | 45 |
| IGKV1-99 | 46 |
| IGKV12-98 | 47 |
| IGKV15-97 | 48 |
| IGKV10-96 | 49 |
| IGKV2-95-2 | 50 |
| IGKV2-95-1 | 51 |
| IGKV10-95 | 52 |
| IGKV10-94 | 53 |
| IGKV2-93-1 | 54 |
| IGKV19-93 | 55 |
| IGKV4-92 | 56 |
| IGKV4-91 | 57 |
| IGKV4-90 | 58 |
| IGKV13-89-1 | 59 |
| IGKV12-89 | 60 |
| IGKV1-88 | 61 |
| IGKV13-87 | 62 |
| IGKV4-86 | 63 |
| IGKV13-85 | 64 |
| IGKV13-84 | 65 |
| IGKV4-83 | 66 |
| IGKV13-82 | 67 |
| IGKV4-81 | 68 |
| IGKV13-80-1 | 69 |
| IGKV4-80 | 70 |
| IGKV4-79 | 71 |
| IGKV13-78-1 | 72 |
| IGKV4-78 | 73 |
| IGKV4-77 | 74 |
| IGKV13-76 | 75 |
| IGKV4-75 | 76 |
| IGKV13-74-1 | 77 |
| IGKV4-74 | 78 |
| IGKV13-73-1 | 79 |
| IGKV4-73 | 80 |
| IGKV4-72 | 81 |
| IGKV13-71-1 | 82 |
| IGKV4-71 | 83 |
| IGKV4-70 | 84 |
| IGKV4-69 | 85 |
| IGKV4-68 | 86 |
| IGKV12-67 | 87 |
| IGKV12-66 | 88 |
| IGKV4-65 | 89 |
| IGKV13-64 | 90 |
| IGKV4-63 | 91 |
| IGKV13-62-1 | 92 |
| IGKV4-62 | 93 |
| IGKV13-61-1 | 94 |
| IGKV4-61 | 95 |
| IGKV4-59 | 96 |
| IGKV4-60 | 97 |
| IGKV4-58 | 98 |
| IGKV13-57-2 | 99 |
| IGKV4-57-1 | 100 |
| IGKV13-57-1 | 101 |
| IGKV4-57 | 102 |
| IGKV13-56-1 | 103 |
| IGKV4-56 | 104 |
| IGKV13-55-1 | 105 |
| IGKV4-55 | 106 |
| IGKV13-54-1 | 107 |
| IGKV4-54 | 108 |
| IGKV4-53 | 109 |
| IGKV4-52 | 110 |
| IGKV4-51 | 111 |
| IGKV4-50 | 112 |
| IGKV12-49 | 113 |
| IGKV5-48 | 114 |
| IGKV12-47 | 115 |
| IGKV12-46 | 116 |
| IGKV5-45 | 117 |
| IGKV12-44 | 118 |
| IGKV5-43 | 119 |
| IGKV12-42 | 120 |
| IGKV12-41 | 121 |
| IGKV5-40-1 | 122 |
| IGKV12-40 | 123 |
| IGKV5-39 | 124 |
| IGKV12-38 | 125 |
| IGKV5-37 | 126 |
| IGKV18-36 | 127 |
| IGKV1-35 | 128 |
| IGKV8-34 | 129 |
| IGKV7-33 | 130 |
| IGKV6-32 | 131 |
| IGKV8-31 | 132 |
| IGKV8-30 | 133 |
| | * |
| IGKV6-29 | 135 |
| IGKV8-28 | 136 |
| IGKV8-27 | 137 |
| IGKV8-26 | 138 |
| IGKV6-25 | 139 |
| IGKV8-24 | 140 |
| IGKV8-23-1 | 141 |
| IGKV6-23 | 142 |
| IGKV8-22 | 143 |
| IGKV8-21 | 144 |
| IGKV6-20 | 145 |
| IGKV8-19 | 146 |
| IGKV8-18 | 147 |
| IGKV6-17 | 148 |
| IGKV8-16 | 149 |
| IGKV6-15 | 150 |
| IGKV6-14 | 151 |
| IGKV6-13 | 152 |
| IGKV3-12-1 | 153 |
| IGKV3-12 | 154 |
| IGKV3-11 | 155 |
| IGKV3-10 | 156 |
| IGKV3-9 | 157 |
| IGKV3-8 | 158 |
| IGKV3-7 | 159 |
| IGKV3-6 | 160 |
| IGKV3-5 | 161 |
| IGKV3-4 | 162 |
| IGKV3-3 | 163 |
| IGKV3-2 | 164 |
| IGKV3-1 | 165 |

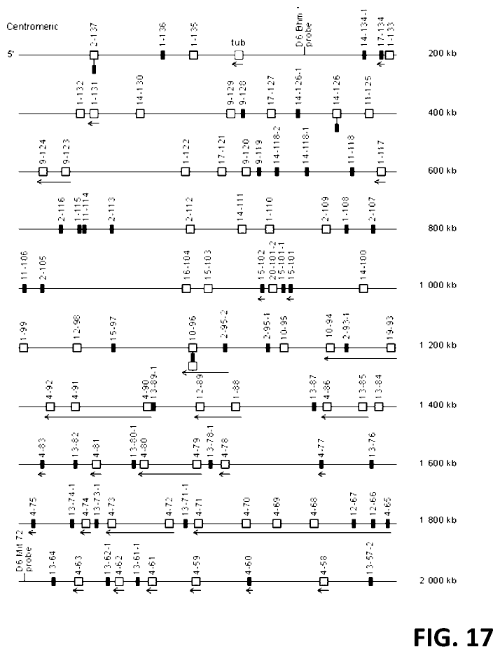

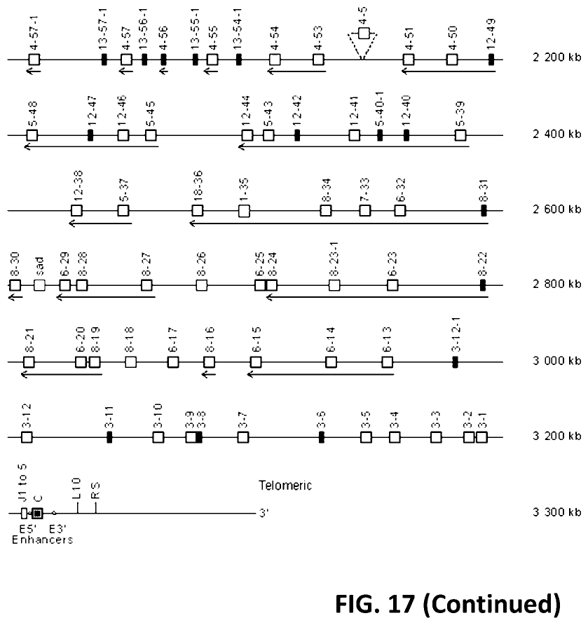

Gm9728 and Amd-ps2 are also located in this locus. The relative order of Gm9728 is 4, and the relative order of Amd-ps2 is 134. Table 10 lists all IGKJ genes and its relative orders on mouse chromosome 6. The IGKC gene, which encodes the light chain immunoglobulin constant domains are after the IGKV and IGKJ genes. These genes and the order of these genes are also shown in FIG. 17.

TABLE 10
List of IGKJ genes on mouse chromosome 6

| Gene names | Order |
|---|---|
| IGKJ1 | 166 |
| IGKJ2 | 167 |
| IGKJ3 | 168 |
| IGKJ4 | 169 |
| IGKJ5 | 170 |

The present disclosure provides genetically-modified, non-human animal comprising one or more human IGKV genes and/or one or more human IGKJ genes. In some embodiments, the human IGKV genes and the human IGKJ genes are operably linked together and can undergo VJ rearrangement. In some embodiments, the human IGKV genes and the human IGKJ genes are at endogenous light chain immunoglobulin gene locus.

In some embodiments, the animal comprises about or at least 1, 2, 3, 4, 5, 6, 7, 8, 9, 10, 20, 30, 40, 50, 60, 61, 62, 63, 64, 65, 66, 67, 68, 69, 70, 71, 72, 73, 74, 75, or 76 human IGKV genes (e.g., genes as shown in Table 7).

In some embodiments, the animal comprises 1, 2, 3, 4, 5, 6, 7, 8, 9, or 10 genes selected from IGKV3D-7, IGKV1D-8, IGKV1D-43, IGKV1D-42, IGKV2D-10, IGKV3D-11, IGKV1D-12, IGKV1D-13, IGKV2D-14, and IGKV3D-15.

In some embodiments, the animal comprises 1, 2, 3, 4, 5, 6, 7, 8, 9, or 10 genes selected from IGKV2-10, IGKV1-9, IGKV1-8, IGKV3-7, IGKV1-6, IGKV1-5, IGKV2-4, IGKV7-3, IGKV5-2, and IGKV4-1.

In some embodiments, the animal comprises about or at least 1, 2, 3, 4, or 5 human IGKJ genes (e.g., genes as shown in Table 3). In some embodiments, the animal comprises 1, 2, 3, 4, or 5 human IGKJ genes selected from IGKJ1, IGKJ2, IGKJ3, IGKJ4, and IGKJ5.

In some embodiments, the animal comprises an endogenous IGKC. In some embodiments, the IGKV genes and/or the IGKJ genes are operably linked together. The VJ recombination can occur among these genes and produce functional antibodies. In some embodiments, these genes are arranged in an order that is similar to the order in human light chain immunoglobulin locus. This arrangement offers various advantages, e.g., the arrangement of these genes allow the production of light chain variable domains with a diversity that is very similar to the diversity of the light chain variable domains in human.

In some embodiments, the IGKV genes and/or the IGKJ genes are operably linked together to the IGKC gene (e.g., endogenous IGKC gene).

In some embodiments, the animal comprises a disruption in the animal's endogenous light chain immunoglobulin gene locus. In some embodiments, the disruption in the animal's endogenous light chain immunoglobulin gene locus comprises a deletion of one or more endogenous IGKV genes, and one or more endogenous IGKJ genes.

In some embodiments, the animal is a mouse. The disruption in the animal's endogenous heavy chain immunoglobulin gene locus comprises a deletion of at least or about 1, 2, 3, 4, 5, 6, 7, 8, 9, 10, 20, 30, 40, 50, 60, 70, 80, 90, 100, 110, 120, 130, 140, 150, 151, 152, 153, 154, 155, 156, 157, 158, 159, 160, 161, 162, or 163 mouse IGKV genes (e.g., genes as shown in Table 9). In some embodiments, the disruption comprises a deletion of about or at least 1, 2, 3, 4, 5, 6, 7, 8, 9, or 10 mouse IGKV genes selected from IGKV2-137, IGKV1-136, IGKV1-135, IGKV14-134-1, IGKV17-134, IGKV1-133, IGKV1-132, IGKV1-131, IGKV14-130, and IGKV9-129. In some embodiments, the mouse still comprises about or at least 1, 2, 3, 4, 5, 6, 7, 8, 9, or 10 mouse IGKV genes selected from IGKV2-137, IGKV1-136, IGKV1-135, IGKV14-134-1, IGKV17-134, IGKV1-133, IGKV1-132, IGKV1-131, IGKV14-130, and IGKV9-129.

In some embodiments, the disruption comprises a deletion of about or at least 1, 2, 3, 4, 5, 6, 7, 8, 9, or 10 mouse IGKV genes selected from IGKV3-10, IGKV3-9, IGKV3-8, IGKV3-7, IGKV3-6, IGKV3-5, IGKV3-4, IGKV3-3, IGKV3-2, and IGKV3-1. In some embodiments, the mouse still comprises about or at least 1, 2, 3, 4, 5, 6, 7, 8, 9, or 10 mouse IGKV genes selected from IGKV3-10, IGKV3-9, IGKV3-8, IGKV3-7, IGKV3-6, IGKV3-5, IGKV3-4, IGKV3-3, IGKV3-2, and IGKV3-1.

In some embodiments, the disruption comprises a deletion of about or at least 1, 2, 3, 4, or 5 mouse IGKJ genes selected from IGKJ1, IGKJ2, IGKJ3, IGKJ4, and IGKJ5. In some embodiments, the mouse still comprises about or at least 1, 2, 3, 4, or 5 mouse IGKJ genes selected from IGKJ1, IGKJ2, IGKJ3, IGKJ4, and IGKJ5 (e.g., IGKJ5).

In some embodiments, the disruption in the animal's endogenous kappa light chain immunoglobulin gene locus comprises a deletion of about or at least 500 kb, 600 kb, 700 kb, 800 kb, 900 kb, 1000 kb, 1500 kb, 2000 kb, 2500 kb, 3000 kb or 3500 kb of an endogenous sequence.

In some embodiments, the deleted sequence starts from IGKV2-137 to IGKJ4, from IGKV1-136 to IGKJ4, from IGKV1-135 to IGKJ4, from IGKV2-137 to IGKJ5, from IGKV1-136 to IGKJ5, or from IGKV1-135 to IGKJ5 (e.g., from IGKV2-137 to IGKJ5).

In some embodiments, the animal comprises about or at least 1, 2, 3, 4, 5, 6, 7, 8, 9, or 10 sequences that are at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence in the human light chain immunoglobulin gene locus. In some embodiments, the sequence has a length of about or at least 10, 20, 30, 40, 50, 60, 70, 80, 90, 100, 200, 300, 400, 500, 600, 700, 800, 900, 1000, 1500, 2000, 2500, 3000 or 3500 kb.

In some embodiments, the animal can have one, two, three, four, five, six, seven, eight, nine, or ten unmodified human sequences. In some embodiments, the unmodified human sequence has a length of about or at least 10, 20, 30, 40, 50, 60, 70, 80, 90, 100, 200, 300, 400, 500, 600, 700, 800, 900, 1000, 1500, 2000, 2500, 3000 or 3500 kb.

In some embodiments, the sequence starts from human IGKV3D-7 to IGKJ5. In some embodiments, the sequence starts from human IGKV3D-7 to IGKJ4. In some embodiments, the sequence starts from human IGKV1D-8 to IGKJ5. In some embodiments, the sequence starts from human IGKV1D-8 to IGKJ4.

The human IGKV genes and the human IGKJ genes are operably linked together and can undergo VJ rearrangement. In some embodiments, the modified mouse has complete human IGKV and IGKJ gene repertoires (e.g., including all non-pseudo human IGKV and IGKJ genes). Thus, the modified mouse can produce a complete human antibody repertory. In some embodiments, after VJ recombination, one IGKV gene (e.g., IGKV1D-43, IGKV1D-13, IGKV1D-16, or IGKV1D-12) contributes to the sequence that encodes an antibody light chain variable region. One human IGKJ gene contributes to the sequence that encodes an antibody light chain variable region. In some embodiments, the IGKV gene is IGKV1D-43, IGKV1D-13, IGKV1D-16, or IGKV1D-12. Furthermore, in some cases, the entire mouse IGKV genes, and IGKJ genes (all none-pseudo genes) are knocked out, and the light chain variable region will not have any sequence that is encoded by a sequence derived from the mouse, thereby minimizing immunogenicity in humans.

In some embodiments, the human proximal Vκ cluster IGKV genes are included in the modified chromosome. In some embodiments, the human distal Vκ cluster IGKV genes are included in the modified chromosome. In some embodiments, both the human proximal Vκ cluster IGKV genes and the human distal Vic cluster IGKV genes are included in the modified chromosome.

### Genetically Modified Lambda Light Chain Immunoglobulin Locus

Lambda chain immunoglobulin locus (also known as IGL or immunoglobulin lambda locus) is a region on the chromosome (e.g., human chromosome 22) that contains genes for the light chains of human antibodies (or immunoglobulins). Similarly, the immunoglobulin light chain genes can also undergo a series rearrangement that lead to the production of a mature immunoglobulin light-chain nucleic acid (e.g., a lambda chain). In a healthy human individual, the total kappa to lambda ratio is roughly 2:1 in serum (measuring intact whole antibodies) or 1:1.5 if measuring free light chains. In mice, the total kappa to lambda ratio is roughly 9:1.

In some embodiments, the animal comprises a human lambda chain immunoglobulin locus.

In some embodiments, the animal comprises a disruption in the animal's endogenous lambda light chain immunoglobulin gene locus. In some embodiments, the disruption in the animal's endogenous light chain immunoglobulin gene locus comprises a deletion of one or more endogenous IGLV genes, one or more endogenous IGLJ genes, and/or one or more immunoglobulin lambda constant (IGLC) genes (e.g., IGLC1, IGLC2, IGLC3, and IGLC4).

The mouse lambda light chain immunoglobulin locus (IGL locus) is located on mouse chromosome 16. Table 11 lists IGLV, IGLJ, and IGLC genes and its relative orders in this locus.

TABLE 11
List of genes at mouse IGL locus

| | | Gene | NCBI | Reference | |
|---|---|---|---|---|---|
| IMGT | Chromosomal | orientation | Gene | GRCm38.p3 | Gene positions in |
| Gene | localization | on chromosome | ID | C57BL/6J | sequence |
| IGLV2 | 16A3 (11.93 cM) | REV | 110612 | NC_000082.6 | 19260403 . . . 19260844 |
| IGLV3 | 16A3 (11.91 cM) | REV | 404743 | NC_000082.6 | 19241208 . . . 19241679 |
| IGLJ2 | 16A3 (11.89 cM) | REV | 404739 | NC_000082.6 | 19200198 . . . 19200235 |
| IGLC2 | 16A3 (11.89 cM) | REV | 110786 | NC_000082.6 | 19198536 . . . 19198852 |
| IGLJ4 | 16A3 (11.89 cM) | REV | 404742 | NC_000082.6 | 19196495 . . . 19196536 |
| IGLC4 | 16A3 (11.89 cM) | REV | 404736 | NC_000082.6 | 19194999 . . . 19195312 |
| IGLV1 | 16A3 (11.82 cM) | REV | 16142 | NC_000082.6 | 19085017 . . . 19085460 |
| IGLJ3 | 16A3 (11.81 cM) | REV | 404740 | NC_000082.6 | 19067041 . . . 19067078 |
| IGLJ3P | 16A3 (11.81 cM) | REV | 404741 | NC_000082.6 | 19066371 . . . 19066408 |
| IGLC3 | 16A3 (11.81 cM) | REV | 110787 | NC_000082.6 | 19065365 . . . 19065681 |
| IGLJ1 | 16A3 (11.81 cM) | REV | 404737 | NC_000082.6 | 19063225 . . . 19063262 |
| IGLC1 | 16A3 (11.80 cM) | REV | 110785 | NC_000082.6 | 19061752 . . . 19062071 |

The disruption in the animal's endogenous lambda light chain immunoglobulin gene locus comprises a deletion of at least or about 1, 2, 3, 4, 5, 6, 7, 8, 9, 10, 11, or 12 mouse IGLV, IGLU, and IGLC genes (e.g., genes as shown in Table 11). In some embodiments, the deletion comprises about or at least 1, 2, 3, or 4 mouse IGKC genes selected from IGLC1, IGLC2, IGLC3, and IGLC4. In some embodiments, the disruption comprises a deletion of about or at least 1, 2, or 3 mouse IGLV genes selected from IGLV1, IGLV2, and IGLV3. In some embodiments, the disruption comprises a deletion of about or at least 1, 2, 3, 4, or 5 mouse IGLJ genes selected from IGLJ1, IGLJ2, IGLJ3, IGLJ3P, and IGLJ4.

In some embodiments, the disruption in the animal's endogenous lambda light chain immunoglobulin gene locus comprises a deletion of about or at least 10 kb, 20 kb, 30 kb, 40 kb, 50 kb, 60 kb, 70 kb, 80 kb, 90 kb, 100 kb, 110 kb, 120 kb, 130 kb, 140 kb, 150 kb, 160 kb, 170 kb, 180 kb, 190 kb, 200 kb, 210 kb, 220 kb, 230 kb, 240 kb, 250 kb, 260 kb, 270 kb, 280 kb, 290 kb, 300 kb, 350 kb, 400 kb, 450 kb, 500 kb, or 1000 kb of nucleotides. In some embodiments, there is no disruption in the animal's endogenous lambda light chain immunoglobulin gene.

In some embodiments, the deleted sequence starts from IGLV2 to IGLC1, from IGLV3 to IGLC1, or from IGLJ2 to IGLC1.

### Major Histocompatibility Complex

The major histocompatibility complex (MHC) plays a key role in the defense mechanism of a body induced by T cell immune responses by presenting a cancer- or virus-derived antigen peptide. MHC is classified as class I or class II. Class I is expressed in all somatic cells except for germ cells and erythrocytes. MHC class I protein complex is composed of an α chain (alpha chain or heavy chain) and a smaller chain known as β2 microglobulin (B2M). The α chain is composed of α1 and α2 domains associated with the formation of an antigen peptide-holding groove and an α3 domain associated with the binding to a co-receptor CD8 molecule expressed on the cytotoxic T cell (CTL) surface. Like MHC class I molecules, class II molecules are also heterodimers, and have two peptides, α and β chains.

Both humans and mice have MHC class I and class II genes. In humans, the classical class I genes are termed HLA-A, HLA-B and HLA-C, whereas in mice they are H-2K, H-2D and H-2L. In Class I molecules, the α-chain (also known as heavy chain) is polymorphic, and the smaller chain B2M (also known as light chain) is generally not polymorphic. The α-chain contains three domains (α1, α2 and α3). Usually, exon 1 of the α-chain gene encodes the leader sequence, exons 2 and 3 encode the α1 and α2 domains, exon 4 encodes the α3 domain, exon 5 encodes the transmembrane domain, and exons 6 and 7 encode the cytoplasmic tail. The α-chain forms a peptide-binding cleft involving the α1 and α2 domains (which resemble Ig-like domains) followed by the α3 domain, which is similar to β2-microglobulin.

Class I MHC are expressed on all nucleated cells, including tumor cells. They are expressed specifically on T and B lymphocytes, macrophages, dendritic cells and neutrophils, among other cells, and function to display peptide fragments (typically 8-10 amino acids in length) on the surface to CD8+ cytotoxic T lymphocytes (CTLs). CTLs are specialized to kill any cell that bears an MHC I-bound peptide recognized by its own membrane-bound TCR. When a cell displays peptides derived from cellular proteins not normally present (e.g., of viral, tumor, or other non-self origin), such peptides are recognized by CTLs, which become activated and kill the cell displaying the peptide.

This disclosure relates to genetically modified animals which express a human or chimeric (e.g., humanized) MHC protein complex, MHC Class I polypeptide, or B2M. As used herein, the term “MHC I complex” or “MHC Class I complex” refers to the complex formed by the MHC I α chain polypeptide and the B2M polypeptide. In some embodiments, the MHC I α chain polypeptide and the B2M polypeptide are fused together. The term “MHC I polypeptide” or “MHC Class I polypeptide,” as used herein, refers to the MHC I α chain polypeptide alone.

### β2 Microglobulin (B2M)

As discussed above, a wildtype MHC class I molecules are heterodimers that consist of two polypeptide chains, α and β2-microglobulin (B2M). The two chains are linked noncovalently via interaction of B2M and the α3 domain. Only the α chain is polymorphic and encoded by a HLA gene. The B2M subunit is not polymorphic and is encoded by the B2M gene. The α3 domain is plasma membrane-spanning and interacts with the CD8 co-receptor of T-cells. The α3-CD8 interaction holds the MHC I molecule in place while the T cell receptor (TCR) on the surface of the cytotoxic T cell binds its α1-α2 heterodimer ligand, and checks the coupled peptide for antigenicity. The α1 and α2 domains fold to make up a groove for peptides to bind. MHC class I molecules bind peptides that are predominantly 8-10 amino acid in length.

B2M (also known as β2M, β₂ microglobulin or beta-2 microglobulin) is a small protein (about 11,800 Dalton), presenting in nearly all nucleated cells and most biological fluids, including serum, urine, and synovial fluid. The human β2M shows 70% amino acid sequence similarity to the murine protein and both of them are located on the syntenic chromosomes. The secondary structure of B2M consists of seven β-strands which are organized into two β-sheets linked by a single disulfide bridge, presenting a classical β-sandwich typical of the immunoglobulin (Ig) domain. B2M has no transmembrane region and contains a distinctive molecular structure called a constant-1 Ig superfamily domain, sharing with other adaptive immune molecules including major histocompatibility complex (MHC) class I and class II. Two evolutionary conserved tryptophan (Trp) residues are important for correct structural fold and function of B2M. Trp60 is exposed to the solvent at the apex of a protein loop and is critical for promoting the association of B2M in MHC I.

Normally, B2M is noncovalently linked with the other polypeptide chain (α chain) to form MHC I or like structures, including MHC I, neonatal Fc receptor (FcRn), a cluster of differentiation 1 (CD1), human hemochromatosis protein (HFE), Qa, and so on. B2M makes extensive contacts with all three domains of the α chain. Thus, the conformation of α chain is highly dependent on the presence of B2M. Although α1 and α2 domains differ among molecules, α3 domain and B2M are relatively conserved, where the intermolecular interaction occurs. A number of residues at the points of contact with B2M are shared among MHC I or like molecules. Furthermore, interactions with α1 and α2 domains are important for the paired association of α3 domain and B2M in the presence of native antigens. B2M can dissociate from such molecules and shed into the serum, where it is transported to the kidneys to be degraded and excreted. An 88-kD protein (calnexin) associates rapidly and quantitatively with newly synthesized murine MHC I molecules within the endoplasmic reticulum. Both B2M and peptide are required for efficient calnexin dissociation and subsequent MHC I transport.

B2M can stabilize the tertiary structure of the MHC I or like molecules. It is also extensively involved in the functional regulation of survival, proliferation, apoptosis, and even metastasis in cancer cells.

A detailed description of B2M and its function can be found, e.g., in Li et al., “The implication and significance of beta 2 microglobulin: A conservative multifunctional regulator.” Chinese Medical Journal 129.4 (2016): 448; and Wang et al., “Targeted Disruption of the β2-Microglobulin Gene Minimizes the Immunogenicity of Human Embryonic Stem Cells,” Stem Cells Translational Medicine 4.10 (2015): 1234-1245; each of which is incorporated herein by reference in its entirety.

In human genomes, B2M gene (Gene ID: 567) locus has four exons, exon 1, exon 2, exon 3, and exon 4. The B2M protein also has a signal peptide. The nucleotide sequence for human B2M mRNA is NM_004048.4, and the amino acid sequence for human B2M is NP_004039.1 (SEQ ID NO: 56). The location for each exon and each region in human B2M nucleotide sequence and amino acid sequence is listed below.

TABLE 12

| | | NP_004039.1 |
|---|---|---|
| Human B2M | NM_004048.3 | 119 aa |
| (approximate location) | 1675 bp | (SEQ ID NO: 56) |
| Exon 1 | 1-97 | 1-22 |
| Exon 2 | 98-376 | 23-115 |
| Exon 3 | 377-404 | 116-119 |
| Exon 4 | 405-1675 | Non-coding |
| Signal peptide | 31-90 | 1-20 |
| B2M excluding signal peptide | 91-387 | 21-119 |

The Human B2M gene (Gene ID: 567) is located in Chromosome 15 of the human genome, which is located from 44,711,487 to 44,718,877, of NC_000015.10 (GRCh38.p13 (GCF_000001405.39)). The 5′-UTR is from 44,711,517 to 44,711,546, exon 1 is from 44,711,614 to 44,711,613, the first intron is from 44,711,614 to 44,715,422, exon 2 is from 44,715,423 to 44,715,701, the second intron is from 44,715,702 to 44,716,328, exon 3 is from 44,716,329 to 44,716,356, the third intron is from 44,716,357 to 44,717,606, exon 4 is from 44,717,607 to 44,718,145, the 3′-UTR is from 44,716,343 to 44,716,356 and 44,717,607 to 44,718,145, based on transcript NM_004048.3. All relevant information for human B2M locus can be found in the NCBI website with Gene ID: 567, which is incorporated by reference herein in its entirety.

In mice, B2M gene locus has four exons, exon 1, exon 2, exon 3, and exon 4. The mouse B2M protein also has a signal peptide. The nucleotide sequence for mouse B2M mRNA is NM_009735.3, the amino acid sequence for mouse B2M is NP_033865.2 (SEQ ID NO: 57). The location for each exon and each region in the mouse B2M nucleotide sequence and amino acid sequence is listed below:

TABLE 13

| Mouse B2M | | NP_033865.2 |
|---|---|---|
| (approximate | NM_009735.3 | 119 aa |
| location) | 858 bp | (SEQ ID NO: 57) |
| Exon 1 | 1-118 | 1-22 |
| Exon 2 | 119-397 | 23-115 |
| Exon 3 | 398-426 | 116-119 |
| Exon 4 | 427-858 | Non-coding |
| Signal peptide | 52-111 | 1-20 |
| B2M excluding signal | 52-426 | 21-119 |
| peptide | | |

The mouse B2M gene (Gene ID: 12010) is located in Chromosome 2 of the mouse genome, which is located from 122,147,686 to 122,153,083, of NC_000068.7 (GRCm38.p6 (GCF_000001635.26)). The 5′-UTR is from 122,147,686 to 122,147,736, exon 1 is from 122,147,686 to 122,147,804, the first intron is from 122,147,805 to 122,150,872, exon 2 is from 122,150,873 to 122,151,151, the second intron is from 122,151,152 to 122,151,646, exon 3 is from 122,151,647 to 122,151,675, the third intron is from 122,151,676 to 122,152,650, exon 4 is from 122,152,651 to 122,153,083, the 3′-UTR is from 122,151,661 to 122,153,083, based on transcript NM_009735.3. All relevant information for mouse B2m locus can be found in the NCBI website with Gene ID: 12010, which is incorporated by reference herein in its entirety.

B2M genes, proteins, and locus of the other species are also known in the art. For example, the gene ID for B2M in Rattus norvegicus (rat) is 24223, the gene ID for B2M in Macaca mulatta (Rhesus monkey) is 712428, the gene ID for B2M in Equus caballus (horse) is 100034203, and the gene ID for B2M in Sus scrofa (pig) is 397033. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) B2M nucleotide sequence and/or amino acid sequences. In some embodiments, the entire sequence of mouse exon 1, exon 2, exon 3, exon 4, and/or signal peptide, are replaced by the corresponding human sequence. In some embodiments, a “region” or “portion” of mouse exon 1, exon 2, exon 3, exon 4, and/or signal peptide, are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, or 110 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, or signal peptide. In some embodiments, a region, a portion, or the entire sequence of mouse exon 1, exon 2, exon 3, and/or exon 4 (e.g., a part of exon 1, exon 2, and a part of exon 3) are replaced by a region, a portion, or the entire sequence of the human exon 1, exon 2, exon 3, and/or exon 4 (e.g., a part of exon 1, exon 2, and a part of exon 3) sequence.

In some embodiments, the present disclosure is related to a genetically-modified, non-human animal whose genome comprises a chimeric (e.g., humanized) B2M nucleotide sequence. In some embodiments, the chimeric (e.g., humanized) B2M nucleotide sequence encodes a B2M protein comprising a signal peptide. In some embodiments, the signal peptide described herein is at least 80%, 85%, 90%, 95%, or 100% identical to amino acids 1-22 of SEQ ID NO: 56. In some embodiments, the signal peptide described herein is at least 80%, 85%, 90%, 95%, or 100% identical to amino acids 1-22 of SEQ ID NO: 57. In some embodiments, the humanized protein has a sequence that is at least 80%, 85%, 90%, 95%, or 100% identical to amino acids 1-119 or 23-119 of SEQ ID NO: 56. In some embodiments, the humanized protein has a sequence that is at least 80%, 85%, 90%, 95%, or 100% identical to amino acids 1-119, 23-119, or 21-119 of SEQ ID NO: 57.

In some embodiments, the present disclosure also provides a chimeric (e.g., humanized) B2M nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse B2M mRNA sequence, mouse B2M amino acid sequence (e.g., SEQ ID NO: 57), or a portion thereof (a portion of exon 1, exon 2, and a portion of exon 3); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human B2M mRNA sequence, human B2M amino acid sequence (e.g., SEQ ID NO: 56, or a portion thereof (e.g., a portion of exon 1, exon 2, and a portion of exon 3).

In some embodiments, the sequence encoding a region of mouse B2M (e.g., amino acids 1-119 of SEQ ID NO: 57) is replaced. In some embodiments, the sequence is replaced by a sequence encoding a corresponding region of human B2M (e.g., amino acids 1-119 of human B2M (SEQ ID NO: 56)).

In some embodiments, the nucleic acids as described herein are operably linked to a promotor or regulatory element, e.g., an endogenous mouse B2M promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse B2M nucleotide sequence (e.g., exon 1, exon 2, exon 3, exon 4, or NM_009735.3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse B2M nucleotide sequence (e.g., exon 1, exon 2, exon 3, exon 4, or NM_009735.3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human B2M nucleotide sequence (e.g., exon 1, exon 2, exon 3, exon 4, or NM_004048.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human B2M nucleotide sequence (e.g., exon 1, exon 2, exon 3, exon 4, or NM_004048.4).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse B2M amino acid sequence (e.g., amino acids encoded by exon 1, exon 2, exon 3, and/or exon 4 of NM_009735.3; or NP_033865.2 (SEQ ID NO: 57)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse B2M amino acid sequence (e.g., amino acids encoded by exon 1, exon 2, exon 3, and/or exon 4 of NM_009735.3; or NP_033865.2 (SEQ ID NO: 57)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human B2M amino acid sequence (e.g., amino acids encoded by exon 1, exon 2, exon 3, and/or exon 4 of NM_004048.4; or NP_004039.1 (SEQ ID NO: 56)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human B2M amino acid sequence (e.g., amino acids encoded by exon 1, exon 2, exon 3, and/or exon 4 of NM_004048.4; or NP_004039.1 (SEQ ID NO: 56)).

### Human Leukocyte Antigen (HLA)

Human leukocyte antigens (HLAs) corresponding to MHC class I molecules include, e.g., HLA-A, HLA-B, and HLA-C. Human HLAs corresponding to MHC class II molecules include, e.g., HLA-DP, HLA-DM, HLA-DO, HLA-DQ, and HLA-DR.

Human HLA-A can have many serotype groups, e.g., HLA-A1 and HLA-A*02. For HLA-A1 (A1), the serotype is determined by the antibody recognition of α1 subset of HLA-A α-chains. For A1, the α chain is encoded by the HLA-A*01 allele group and the β-chain is encoded by B2M locus. This group currently is dominated by A*0101 (A*01:01:01:01). For HLA-A*02 (HLA-A2), the serotype is determined by the antibody recognition of the α2 domain of the HLA-A α-chain. For A*02, the α chain is encoded by the HLA-A*02 gene and the 0 chain is encoded by the B2M locus. A subtype of HLA-A2 is HLA-A2.1. Details of HLA nomenclature can be found, e.g., in Marsh, S. G. et al., “Nomenclature for factors of the HLA system, 2010.” Tissue antigens 75.4 (2010): 291, which is incorporated herein by reference in its entirety.

In human genomes, a typical HLA-A gene locus has eight exons, exon 1, exon 2, exon 3, exon 4, exon 5, exon 6, exon 7, and exon 8. The HLA protein also has a signal peptide, an extracellular region, a transmembrane region, and a cytoplasmic region. Further, the extracellular region includes an α1 domain, an α2 domain, an α3 domain, and a connecting peptide. The nucleotide sequence for human HLA-A*0101 mRNA is NM_001242758.1, and the amino acid sequence for human HLA-A*0101 is NP_001229687.1 (SEQ ID NO: 58). In addition, the nucleotide sequence for human HLA-A is nucleic acids 95493-99436 of AF055066.1, and the amino acid sequence for human HLA-A is AAC24825.1 (SEQ ID NO: 59). The location for each exon and each region in human HLA-A nucleotide sequence and amino acid sequence is listed below.

TABLE 14

| | | HLA-A*0101 | |
|---|---|---|---|
| | | NP_{—} | HLA-A |
| | | 001229687.1 | AAC24825.1 |
| Human HLA-A | NM_{—} | 365 aa | 362aa |
| (approximate | 001242758.1 | (SEQ ID | (SEQ ID |
| location) | 1611 bp | NO: 58) | NO: 59) |
| Exon 1 | 1-157 | 1-24 | 1-21 |
| Exon 2 | 158-427 | 25-114 | 22-111 |
| Exon 3 | 428-703 | 115-206 | 112-203 |
| Exon 4 | 704-979 | 207-298 | 204-295 |
| Exon 5 | 980-1096 | 299-337 | 296-334 |
| Exon 6 | 1097-1129 | 338-348 | 335-345 |
| Exon 7 | 1130-1177 | 349-364 | 346-362 |
| Exon 8 | 1178-1611 | 365 | Non-coding |
| Signal peptide | 85-156 | 1-24 | 1-21 |
| Extracellular | 157-1008 | 25-308 | 22-305 |
| Transmembrane | 1009-1080 | 309-332 | 306-329 |
| Cytoplasmic | 1081-1611 | 333-365 | 330-362 |
| Alpha-1 | 157-426 | 25-114 | 22-111 |
| Alpha-2 | 427-702 | 115-206 | 112-203 |
| Alpha-3 | 703-978 | 207-298 | 204-295 |
| Connecting | 979-1008 | 299-308 | 296-305 |
| peptide | | | |

Human MHC class I region (GenBank ID: AF055066.1) is located in Chromosome 6 of the human genome. The Human HLA-A gene is located from 95493 to 99436 of AF055066.1. Exon 1 is from 99566 to 99629, the first intron is from 99436 to 99565, exon 2 is from 99166 to 99435, the second intron is from 98925 to 99165, exon 3 is from 98649 to 98924, the third intron is from 98049 to 98648, exon 4 is from 97773 to 98048, the fourth intron is from 97674 to 97772, exon 5 is from 97557 to 97673, the fifth intron is from 97119 to 97556, exon 6 is from 97086 to 97118, the sixth intron is from 97085 to 96944, exon 7 is from 96896 to 96943, the seventh intron is from 96727 to 96895, exon 8 is from 96726 to 96322, the 3′-UTR is from 96721 to 96322, based on AF055066.1. All relevant information for human HLA-A gene locus can be found in the NCBI website with GenBank ID: AF055066.1, which is incorporated by reference herein in its entirety.

In some embodiments, the HLA described herein is HLA-A (e.g., HLA-A*0101, HLA-A*0201, HLA-A*0301, HLA-A*0302, HLA-A*1101, HLA-A*2402, HLA-A*2901, HLA-A*3101, HLA-A*3201, HLA-A*3301, or HLA-A*3303), HLA-B (e.g., HLA-B*4402, or HLA-B*0702), or HLA-C(e.g., HLA-C*0702, HLA-C*0102, HLA-C*0701, HLA-C*0401, HLA-C*0801, or HLA-C*0802).

HLA-A*0101 has an amino acid sequence set forth in SEQ ID NO: 58. Specifically, the signal peptide of HLA-A*0101 corresponds to position 1 to position 24 of SEQ ID NO: 58, the α1 domain corresponds to position 25 to position 114 of SEQ ID NO: 58, the α2 domain corresponds to position 115 to position 206 of SEQ ID NO: 58, the α3 domain corresponds to position 207 to position 298 of SEQ ID NO: 58, the connecting peptide corresponds to position 299 to position 308 of SEQ ID NO: 58, the transmembrane region corresponds to position 309 to position 332 of SEQ ID NO: 58, and the cytoplasmic region corresponds to position 333 to position 365 of SEQ ID NO: 58.

HLA-A*0201 has an amino acid sequence set forth in SEQ ID NO: 6. Specifically, the signal peptide of HLA-A*0201 corresponds to position 1 to position 24 of SEQ ID NO: 6, the α1 domain corresponds to position 25 to position 114 of SEQ ID NO: 6, the α2 domain corresponds to position 115 to position 206 of SEQ ID NO: 6, the α3 domain corresponds to position 207 to position 298 of SEQ ID NO: 6, the connecting peptide corresponds to position 299 to position 308 of SEQ ID NO: 6, the transmembrane region corresponds to position 309 to position 332 of SEQ ID NO: 6, and the cytoplasmic region corresponds to position 333 to position 365 of SEQ ID NO: 6.

HLA-A*0301 has an amino acid sequence set forth in SEQ ID NO: 7. Specifically, the signal peptide of HLA-A*0301 corresponds to position 1 to position 24 of SEQ ID NO: 7, the α1 domain corresponds to position 25 to position 114 of SEQ ID NO: 7, the α2 domain corresponds to position 115 to position 206 of SEQ ID NO: 7, the α3 domain corresponds to position 207 to position 298 of SEQ ID NO: 7, the connecting peptide corresponds to position 299 to position 308 of SEQ ID NO: 7, the transmembrane region corresponds to position 309 to position 332 of SEQ ID NO: 7, and the cytoplasmic region corresponds to position 333 to position 365 of SEQ ID NO: 7.

HLA-A*0302 has an amino acid sequence set forth in SEQ ID NO: 8. Specifically, the signal peptide of HLA-A*0302 corresponds to position 1 to position 24 of SEQ ID NO: 8, the α1 domain corresponds to position 25 to position 114 of SEQ ID NO: 8, the α2 domain corresponds to position 115 to position 206 of SEQ ID NO: 8, the 3 domain corresponds to position 207 to position 298 of SEQ ID NO: 8, the connecting peptide corresponds to position 299 to position 308 of SEQ ID NO: 8, the transmembrane region corresponds to position 309 to position 332 of SEQ ID NO: 8, and the cytoplasmic region corresponds to position 333 to position 365 of SEQ ID NO: 8.

HLA-A*1101 has an amino acid sequence set forth in SEQ ID NO: 9. Specifically, the signal peptide of HLA-A*1101 corresponds to position 1 to position 24 of SEQ ID NO: 9, the α1 domain corresponds to position 25 to position 114 of SEQ ID NO: 9, the α2 domain corresponds to position 115 to position 206 of SEQ ID NO: 9, the α3 domain corresponds to position 207 to position 298 of SEQ ID NO: 9, the connecting peptide corresponds to position 299 to position 308 of SEQ ID NO: 9, the transmembrane region corresponds to position 309 to position 332 of SEQ ID NO: 9, and the cytoplasmic region corresponds to position 333 to position 365 of SEQ ID NO: 9.

HLA-A*2402 has an amino acid sequence set forth in SEQ ID NO: 10. Specifically, the signal peptide of HLA-A*2402 corresponds to position 1 to position 24 of SEQ ID NO: 10, the α1 domain corresponds to position 25 to position 114 of SEQ ID NO: 10, the α2 domain corresponds to position 115 to position 206 of SEQ ID NO: 10, the α3 domain corresponds to position 207 to position 298 of SEQ ID NO: 10, the connecting peptide corresponds to position 299 to position 308 of SEQ ID NO: 10, the transmembrane region corresponds to position 309 to position 332 of SEQ ID NO: 10, and the cytoplasmic region corresponds to position 333 to position 365 of SEQ ID NO: 10.

HLA-A*2901 has an amino acid sequence set forth in SEQ ID NO: 11. Specifically, the signal peptide of HLA-A*2901 corresponds to position 1 to position 24 of SEQ ID NO: 11, the α1 domain corresponds to position 25 to position 114 of SEQ ID NO: 11, the α2 domain corresponds to position 115 to position 206 of SEQ ID NO: 11, the α3 domain corresponds to position 207 to position 298 of SEQ ID NO: 11, the connecting peptide corresponds to position 299 to position 308 of SEQ ID NO: 11, the transmembrane region corresponds to position 309 to position 332 of SEQ ID NO: 11, and the cytoplasmic region corresponds to position 333 to position 365 of SEQ ID NO: 11.

HLA-A*3101 has an amino acid sequence set forth in SEQ ID NO: 12. Specifically, the signal peptide of HLA-A*3101 corresponds to position 1 to position 24 of SEQ ID NO: 12, the α1 domain corresponds to position 25 to position 114 of SEQ ID NO: 12, the α2 domain corresponds to position 115 to position 206 of SEQ ID NO: 12, the α3 domain corresponds to position 207 to position 298 of SEQ ID NO: 12, the connecting peptide corresponds to position 299 to position 308 of SEQ ID NO: 12, the transmembrane region corresponds to position 309 to position 332 of SEQ ID NO: 12, and the cytoplasmic region corresponds to position 333 to position 365 of SEQ ID NO: 12.

HLA-A*3201 has an amino acid sequence set forth in SEQ ID NO: 13. Specifically, the signal peptide of HLA-A*3201 corresponds to position 1 to position 24 of SEQ ID NO: 13, the α1 domain corresponds to position 25 to position 114 of SEQ ID NO: 13, the α2 domain corresponds to position 115 to position 206 of SEQ ID NO: 13, the α3 domain corresponds to position 207 to position 298 of SEQ ID NO: 13, the connecting peptide corresponds to position 299 to position 308 of SEQ ID NO: 13, the transmembrane region corresponds to position 309 to position 332 of SEQ ID NO: 13, and the cytoplasmic region corresponds to position 333 to position 365 of SEQ ID NO: 13.

HLA-A*3301 has an amino acid sequence set forth in SEQ ID NO: 14. Specifically, the signal peptide of HLA-A*3301 corresponds to position 1 to position 24 of SEQ ID NO: 14, the α1 domain corresponds to position 25 to position 114 of SEQ ID NO: 14, the α2 domain corresponds to position 115 to position 206 of SEQ ID NO: 14, the α3 domain corresponds to position 207 to position 298 of SEQ ID NO: 14, the connecting peptide corresponds to position 299 to position 308 of SEQ ID NO: 14, the transmembrane region corresponds to position 309 to position 332 of SEQ ID NO: 14, and the cytoplasmic region corresponds to position 333 to position 365 of SEQ ID NO: 14.

HLA-C*0802 has an amino acid sequence set forth in SEQ ID NO: 15. Specifically, the signal peptide of HLA-C*0802 corresponds to position 1 to position 24 of SEQ ID NO: 15, the α1 domain corresponds to position 25 to position 114 of SEQ ID NO: 15, the α2 domain corresponds to position 115 to position 206 of SEQ ID NO: 15, the α3 domain corresponds to position 207 to position 298 of SEQ ID NO: 15, the connecting peptide corresponds to position 299 to position 308 of SEQ ID NO: 15, the transmembrane region corresponds to position 309 to position 333 of SEQ ID NO: 15, and the cytoplasmic region corresponds to position 334 to position 366 of SEQ ID NO: 15.

HLA-A*3303 has an amino acid sequence set forth in SEQ ID NO: 92. Specifically, the signal peptide of HLA-A*3303 corresponds to position 1 to position 24 of SEQ ID NO: 92, the α1 domain corresponds to position 25 to position 114 of SEQ ID NO: 92, the α2 domain corresponds to position 115 to position 206 of SEQ ID NO: 92, the α3 domain corresponds to position 207 to position 298 of SEQ ID NO: 92, the connecting peptide corresponds to position 299 to position 308 of SEQ ID NO: 92, the transmembrane region corresponds to position 309 to position 332 of SEQ ID NO: 92, and the cytoplasmic region corresponds to position 333 to position 365 of SEQ ID NO: 92.

HLA-B*4402 has an amino acid sequence set forth in SEQ ID NO: 93. Specifically, the signal peptide of HLA-B*4402 corresponds to position 1 to position 24 of SEQ ID NO: 93, the α1 domain corresponds to position 25 to position 114 of SEQ ID NO: 93, the α2 domain corresponds to position 115 to position 206 of SEQ ID NO: 93, the α3 domain corresponds to position 207 to position 298 of SEQ ID NO: 93, the connecting peptide corresponds to position 299 to position 308 of SEQ ID NO: 93, the transmembrane region corresponds to position 309 to position 332 of SEQ ID NO: 93, and the cytoplasmic region corresponds to position 333 to position 362 of SEQ ID NO: 93.

HLA-B*0702 has an amino acid sequence set forth in SEQ ID NO: 94. Specifically, the signal peptide of HLA-B*0702 corresponds to position 1 to position 24 of SEQ ID NO: 94, the α1 domain corresponds to position 25 to position 114 of SEQ ID NO: 94, the α2 domain corresponds to position 115 to position 206 of SEQ ID NO: 94, the α3 domain corresponds to position 207 to position 298 of SEQ ID NO: 94, the connecting peptide corresponds to position 299 to position 308 of SEQ ID NO: 94, the transmembrane region corresponds to position 309 to position 332 of SEQ ID NO: 94, and the cytoplasmic region corresponds to position 333 to position 362 of SEQ ID NO: 94.

HLA-C*0702 has an amino acid sequence set forth in SEQ ID NO: 95. Specifically, the signal peptide of HLA-C*0702 corresponds to position 1 to position 24 of SEQ ID NO: 95, the α1 domain corresponds to position 25 to position 114 of SEQ ID NO: 95, the α2 domain corresponds to position 115 to position 206 of SEQ ID NO: 95, the α3 domain corresponds to position 207 to position 298 of SEQ ID NO: 95, the connecting peptide corresponds to position 299 to position 308 of SEQ ID NO: 95, the transmembrane region corresponds to position 309 to position 333 of SEQ ID NO: 95, and the cytoplasmic region corresponds to position 334 to position 366 of SEQ ID NO: 95.

HLA-C*0102 has an amino acid sequence set forth in SEQ ID NO: 96. Specifically, the signal peptide of HLA-C*0102 corresponds to position 1 to position 24 of SEQ ID NO: 96, the α1 domain corresponds to position 25 to position 114 of SEQ ID NO: 96, the α2 domain corresponds to position 115 to position 206 of SEQ ID NO: 96, the α3 domain corresponds to position 207 to position 298 of SEQ ID NO: 96, the connecting peptide corresponds to position 299 to position 308 of SEQ ID NO: 96, the transmembrane region corresponds to position 309 to position 333 of SEQ ID NO: 96, and the cytoplasmic region corresponds to position 334 to position 366 of SEQ ID NO: 96.

HLA-C*0701 has an amino acid sequence set forth in SEQ ID NO: 97. Specifically, the signal peptide of HLA-C*0701 corresponds to position 1 to position 24 of SEQ ID NO: 97, the α1 domain corresponds to position 25 to position 114 of SEQ ID NO: 97, the α2 domain corresponds to position 115 to position 206 of SEQ ID NO: 97, the α3 domain corresponds to position 207 to position 298 of SEQ ID NO: 97, the connecting peptide corresponds to position 299 to position 308 of SEQ ID NO: 97, the transmembrane region corresponds to position 309 to position 333 of SEQ ID NO: 97, and the cytoplasmic region corresponds to position 334 to position 366 of SEQ ID NO: 97.

HLA-C*0401 has an amino acid sequence set forth in SEQ ID NO: 98. Specifically, the signal peptide of HLA-C*0401 corresponds to position 1 to position 24 of SEQ ID NO: 98, the α1 domain corresponds to position 25 to position 114 of SEQ ID NO: 98, the α2 domain corresponds to position 115 to position 206 of SEQ ID NO: 98, the α3 domain corresponds to position 207 to position 298 of SEQ ID NO: 98, the connecting peptide corresponds to position 299 to position 308 of SEQ ID NO: 98, the transmembrane region corresponds to position 309 to position 333 of SEQ ID NO: 98, and the cytoplasmic region corresponds to position 334 to position 366 of SEQ ID NO: 98.

HLA-C*0801 has an amino acid sequence set forth in SEQ ID NO: 99. Specifically, the signal peptide of HLA-C*0801 corresponds to position 1 to position 24 of SEQ ID NO: 99, the α1 domain corresponds to position 25 to position 114 of SEQ ID NO: 99, the α2 domain corresponds to position 115 to position 206 of SEQ ID NO: 99, the α3 domain corresponds to position 207 to position 298 of SEQ ID NO: 99, the connecting peptide corresponds to position 299 to position 308 of SEQ ID NO: 99, the transmembrane region corresponds to position 309 to position 333 of SEQ ID NO: 99, and the cytoplasmic region corresponds to position 334 to position 366 of SEQ ID NO: 99.

In mice, H-2K, H-2D and H-2L are MHC class I genes. Particularly, the H2-D1 gene locus has eight exons, exon 1, exon 2, exon 3, exon 4, exon 5, exon 6, exon 7 and exon 8. The mouse H2-D1 protein (encoding MHC class I α chain) also has a signal peptide, an extracellular region, a transmembrane region, and a cytoplasmic region. Specifically, the extracellular region includes an α1 domain, an α2 domain, an α3 domain, and a connecting peptide. The nucleotide sequence for mouse H2-D1 mRNA is NM_010380.3, the amino acid sequence for mouse H2-D1 is NP_034510.3 (SEQ ID NO: 60). The location for each exon and each region in the mouse H2-D1 nucleotide sequence and amino acid sequence is listed below:

TABLE 15

| Mouse H2-D1 | | NP_034510.3 |
|---|---|---|
| (approximate | NM_010380.3 | 362aa |
| location) | 1736 bp | (SEQ ID NO: 60) |
| Exon 1 | 1-93 | 1-24 |
| Exon 2 | 94-363 | 25-114 |
| Exon 3 | 364-639 | 115-206 |
| Exon 4 | 640-915 | 207-298 |
| Exon 5 | 916-1032 | 299-337 |
| Exon 6 | 1033-1065 | 338-348 |
| Exon 7 | 1066-1104 | 349-361 |
| Exon 8 | 1105-1736 | 362 |
| Signal peptide | 21-92 | 1-24 |
| Extracellular | 93-947 | 25-309 |
| Transmembrane | 948-1013 | 310-331 |
| Cytoplasmic | 1014-1736 | 332-362 |
| Alpha-1 | 93-362 | 25-114 |
| Alpha-2 | 363-638 | 115-206 |
| Alpha-3 | 639-914 | 207-298 |
| Connecting peptide | 915-947 | 299-309 |

The mouse H2-D1 gene (Gene ID: 14964; MGI: 95896) is located in Chromosome 17 of the mouse genome, which is located from 35482070 to 35486473 of NC_000083.7 (GRCm39 (GCF_000001635.27)). The 5′-UTR is from 35,262,730 to 35,263,113, exon 1 is from 35,262,730 to 35,263,186, the first intron is from 35,263,187 to 35,263,378, exon 2 is from 35,263,379 to 35,263,648, the second intron is from 35,263,649 to 35,263,838, exon 3 is from 35,263,839 to 35,264,114, the third intron is from 35,264,115 to 35,265,783, exon 4 is from 35,265,784 to 35,266,059, the forth intron is from 35,266,060 to 35,266,186, exon 5 is from 35,266,187 to 35,266,303, the fifth intron is from 35,266,304 to 35,266,481, exon 6 is from 35,266,482 to 35,266,514, the sixth intron is from 35,266,515 to 35,266,687, exon 7 is from 35,266,688 to 35,266,726, the seventh intron is from 35,266,727 to 35,266,865, exon 8 is from 35,266,866 to 35,267,499, the 3′-UTR is from 35,266,871 to 35,267,499, based on transcript NM_010380.3. All relevant information for mouse H2-D1 locus can be found in the NCBI website with Gene ID: 14964, which is incorporated by reference herein in its entirety.

Mouse H2-D1 has an amino acid sequence set forth in SEQ ID NO: 60. Specifically, the signal peptide of mouse H2-D1 corresponds to position 1 to position 24 of SEQ ID NO: 60, the α1 domain corresponds to position 25 to position 114 of SEQ ID NO: 60, the α2 domain corresponds to position 115 to position 206 of SEQ ID NO: 60, the α3 domain corresponds to position 207 to position 298 of SEQ ID NO: 60, the connecting peptide corresponds to position 299 to position 309 of SEQ ID NO: 60, the transmembrane region corresponds to position 310 to position 3331 of SEQ ID NO: 60, and the cytoplasmic region corresponds to position 332 to position 362 of SEQ ID NO: 60.

MHC molecule genes, proteins, and locus of the other species are also known in the art. For example, the gene ID and the relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) MHC molecule (e.g., MHC class I alpha chain) nucleotide sequence and/or amino acid sequences. This disclosure also relates to genetically modified animals which express a human or chimeric (e.g., humanized) HLA protein complex and/or HLA polypeptide. As used herein, the term “HLA complex” or “HLA protein complex” refers to the complex formed by the HLA α chain polypeptide and the B2M polypeptide. In some embodiments, the HLA α chain polypeptide and the B2M polypeptide are fused together. The term “HLA” or “HLA polypeptide” as used herein refers to the HLA α chain polypeptide.

In some embodiments, the entire sequence of mouse H2-D1 exon 1, exon 2, exon 3, exon 4, exon 5, exon 6, exon 7, exon 8, signal peptide, extracellular region (e.g., α1 domain, α2 domain, α3 domain, and/or connecting peptide), transmembrane region, and/or cytoplasmic region are replaced by the corresponding human sequence. In some embodiments, a “region” or “portion” of mouse H2-D1 exon 1, exon 2, exon 3, exon 4, exon 5, exon 6, exon 7, exon 8, signal peptide, extracellular region (e.g., α1 domain, α2 domain, 3 domain, and/or connecting peptide), transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, or 360 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, signal peptide, extracellular region (e.g., α1 domain, α2 domain, α3 domain, and/or connecting peptide), transmembrane region, and/or cytoplasmic region. In some embodiments, a region, a portion, or the entire sequence of mouse H2-D1 exon 1, exon 2, exon 3, exon 4, exon 5, exon 6, exon 7, and/or exon 8 (e.g., exon 1, exon 2, and exon 3) are replaced by a region, a portion, or the entire sequence of the human HLA (e.g., HLA-A, HLA-B, or HLA-C) exon 1, exon 2, exon 3, exon 4, exon 5, exon 6, exon 7, and/or exon 8 (e.g., exon 1, exon 2, exon 3) sequence. In some embodiments, the HLA described herein comprises an amino acid sequence that is at least 80%, 85%, 90%, 91%, 92%, 93%, 94%, 95%, 96%, 97%, 98%, or at least 99% identical to SEQ ID NO: 58, 59, 6, 7, 8, 9, 10, 11, 12, 13, 14, 15, 92, 93, 94, 95, 96, 97, 98, or 99.

In some embodiments, the present disclosure is related to a genetically-modified, non-human animal whose genome comprises a chimeric (e.g., humanized) MHC molecule (e.g., human HLA/mouse H2-D1) nucleotide sequence. In some embodiments, the chimeric (e.g., humanized) MHC molecule nucleotide sequence encodes a MHC molecule protein comprising an extracellular region, a transmembrane region, a cytoplasmic region, and a signal peptide. In some embodiments, the extracellular region comprises the entire or part of human HLA (e.g., HLA-A*0101, HLA-A, or any of the HLA molecules described herein) extracellular region. For example, the extracellular region described herein comprises an amino acid sequence that is at least 80%, 85%, 90%, 95%, or 100% identical to human HLA extracellular region (e.g., amino acids 25-308 of SEQ ID NO: 58, or amino acids 22-305 of SEQ ID NO: 59). In some embodiments, the transmembrane region comprises the entire or part of human HLA (e.g., HLA-A*0101, HLA-A, or any of the HLA molecules described herein) transmembrane region. For example, the transmembrane region is at least 80%, 85%, 90%, 95%, or 100% identical to human HLA transmembrane region (e.g., amino acids 309-332 of SEQ ID NO: 58, or amino acids 306-329 of SEQ ID NO: 59). In some embodiments, the cytoplasmic region comprises the entire or part of human HLA (e.g., HLA-A*0101, HLA-A, or any of the HLA molecules described herein) cytoplasmic region. For example, the cytoplasmic region is at least 80%, 85%, 90%, 95%, or 100% identical to human HLA cytoplasmic region (e.g., amino acids 333-365 of SEQ ID NO: 58, or amino acids 330-362 of SEQ ID NO: 59).

In some embodiments, the chimeric (e.g., humanized) MHC molecule nucleotide sequence encodes a MHC molecule protein comprising a signal peptide. In some embodiments, the signal peptide described herein is at least 80%, 85%, 90%, 95%, or 100% identical to amino acids 1-24 of SEQ ID NO: 60. In some embodiments, the signal peptide described herein is at least 80%, 85%, 90%, 95%, or 100% identical to amino acids 1-24 of SEQ ID NO: 58, or amino acids 1-21 of SEQ ID NO: 59. In some embodiments, the signal peptide described herein is at least 80%, 85%, 90%, 95%, or 100% identical to the signal peptide of any HLA molecules described herein.

In some embodiments, the present disclosure also provides a chimeric (e.g., humanized) MHC molecule nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse H2-D1 mRNA sequence, mouse H2-D1 amino acid sequence (e.g., SEQ ID NO: 60), or a portion thereof (e.g., exon 4, exon 5, exon 6, exon 7, and a portion of exon 8); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human HLA molecule mRNA sequence, human HLA amino acid sequence (e.g., SEQ ID NO: 58, SEQ ID NO: 59, or any one of SEQ ID NOs: 6-15 and 92-99), or a portion thereof (e.g., a portion of exon 1, exon 2, and exon 3).

In some embodiments, the sequence encoding a region of mouse H2-D1 (e.g., amino acids 1-206 or 25-206 of SEQ ID NO: 60) is replaced. In some embodiments, the sequence is replaced by a sequence encoding a corresponding region of human HLA (e.g., amino acids 1-206 or 25-206 of human HLA-A*0101 (SEQ ID NO: 58); or amino acids 1-203 or 22-203 of human HLA-A (SEQ ID NO: 59)).

In some embodiments, the nucleic acids as described herein are operably linked to a promotor or regulatory element, e.g., an endogenous mouse H2-D1 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse H2-D1 nucleotide sequence (e.g., exon 1, exon 2, exon 3, exon 4, exon 5, exon 6, exon 7, exon 8, or NM_010380.3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse H2-D1 nucleotide sequence (e.g., exon 1, exon 2, exon 3, exon 4, exon 5, exon 6, exon 7, exon 8, or NM_010380.3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human HLA nucleotide sequence (e.g., exon 1, exon 2, exon 3, exon 4, exon 5, exon 6, exon 7, exon 8, or NM_001242758.1).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human HLA nucleotide sequence (e.g., exon 1, exon 2, exon 3, exon 4, exon 5, exon 6, exon 7, exon 8, or NM_001242758.1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse H2-D1 amino acid sequence (e.g., amino acids encoded by exon 1, exon 2, exon 3, exon 4, exon 5, exon 6, exon 7, and/or exon 8 of NM_010380.3; or NP_034510.3 (SEQ ID NO: 60)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse H2-D1 amino acid sequence (e.g., amino acids encoded by exon 1, exon 2, exon 3, exon 4, exon 5, exon 6, exon 7, and/or exon 8 of NM_010380.3; or NP_034510.3 (SEQ ID NO: 60)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human HLA amino acid sequence (e.g., amino acids encoded by exon 1, exon 2, exon 3, exon 4, exon 5, exon 6, exon 7, and/or exon 8 of NM_001242758.1; NP_001229687.1 (SEQ ID NO: 58); or AAC24825.1 (SEQ ID NO: 59)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human HLA amino acid sequence (e.g., amino acids encoded by exon 1, exon 2, exon 3, exon 4, exon 5, exon 6, exon 7, and/or exon 8 of NM_001242758.1; NP_001229687.1 (SEQ ID NO: 58); or AAC24825.1 (SEQ ID NO: 59)).

The present disclosure further relates to a B2M or MHC molecule genomic DNA sequence of a humanized mouse. The DNA sequence is obtained by reverse transcription of the mRNA obtained by transcription thereof is consistent with or complementary to the DNA sequence homologous to the sequence as described herein.

The disclosure also provides an amino acid sequence that has a homology of at least 90% with, or at least 90% identical to the sequence shown in any one of SEQ ID NOs: 61-80 and 100-117, and has protein activity. In some embodiments, the homology with the sequence shown in any one of SEQ ID NOs: 61-80 and 100-117 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in any one of SEQ ID NOs: 61-80 and 100-117 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) MHC from an endogenous non-human B2M or MHC gene locus.

### Genetically Modified Animals

As used herein, the term “genetically-modified non-human animal” refers to a non-human animal having a modified sequence in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the modified sequence in its genome. The cell having the modified sequence can be various kinds of cells, e.g., an endogenous cell, a somatic cell, an immune cell, a T cell, a B cell, a germ cell, a blastocyst, or an endogenous tumor cell. In some embodiments, genetically-modified non-human animals are provided that comprise a human or humanized B2M and/or human or humanized MHC molecule gene (e.g., MHC class I α chain) at the endogenous B2M or MHC gene locus. The animals are generally able to pass the modification to progeny, i.e., through germline transmission.

As used herein, the term “humanized” and the like refers to a molecule (e.g., a nucleic acid, protein, etc.) that was non-human in origin and for which a portion has been replaced with a corresponding portion of a corresponding human molecule in such a manner that the modified (e.g., humanized) molecule retains its biological function and/or maintains the structure that performs the retained biological function. A humanized molecule may be considered derived from a human molecule where the humanized molecule is encoded by a nucleotide comprising a nucleic acid sequence that encodes the human molecule (or a portion thereof).

In some embodiments, the genetically-modified non-human animal does not express an endogenous B2M (e.g., mouse B2M). In some embodiments, the genetically-modified non-human animal does not express a functional endogenous B2M (e.g., mouse B2M). In some embodiments, the genetically-modified non-human animal does not express an endogenous MHC molecule (e.g., mouse H2-D1). In some embodiments, the genetically-modified non-human animal does not express a functional endogenous MHC molecule (e.g., mouse H2-D1) or a functional endogenous MHC protein complex.

In some embodiments, the genetically-modified non-human animal described herein is immunodeficient. In some embodiments, the animal has a NOD-Prkdc^{scid} IL-2rγ^{nu1}, NOD-Rag 1^{-/-}-IL2rg^{-/-} (NRG), Rag 2^{-/-}-IL2rg^{-/-} (RG), or NOD/SCID (NOD-Prkdc^{scid}) background.

In some embodiments, the genetically-modified non-human animal described herein (e.g., mouse) have a disrupted endogenous B2M gene. In some embodiments, the genetically-modified non-human animal described herein (e.g., mouse) expresses a dysfunctional endogenous B2M protein (e.g., mouse B2M). In some embodiments, the genetically-modified non-human animal described herein (e.g., mouse) have a disrupted endogenous MHC gene. In some embodiments, the genetically-modified non-human animal described herein (e.g., mouse) expresses a dysfunctional endogenous MHC molecule (e.g., mouse H2-D1) or a dysfunctional endogenous MHC protein complex.

In some embodiments, the genetically-modified non-human animal is a RenMab™ mouse (hVH/hVL mice), which have both a humanized immunoglobulin heavy chain locus and a humanized immunoglobulin kappa chain locus. Detailed descriptions of RenMab™ mice can be found, e.g., in PCT/CN2020/075698 and US20200390073A1, which are incorporated herein by reference in the entirety.

In some embodiments, the genetically-modified non-human animal is a RenLite™ mouse (or hVH/hcVL mice), which have a humanized immunoglobulin heavy chain locus and a humanized common light chain locus. Detailed descriptions of RenLite™ mice can be found, e.g., in PCT/CN2021/097652, which is incorporated herein by reference in its entirety.

In some embodiments, the genetically-modified non-human animal is a mouse. In some embodiments, the genetically-modified non-human animal is a B-NDG mouse. Details of B-NDG mice can be found, e.g., in PCT/CN2018/079365; U.S. Ser. No. 10/820,580B2, each of which is incorporated herein by reference in its entirety. In some embodiments, the genetically modified animal is a NSG mouse or NOG mouse. A detailed description of the NSG mice and NOD mice can be found, e.g., in Ishikawa et al. “Development of functional human blood and immune systems in NOD/SCID/IL2 receptor γ chainnull mice.” Blood 106.5 (2005): 1565-1573; Katano et al. “NOD-Rag2null IL-2Rγnull mice: an alternative to NOG mice for generation of humanized mice.” Experimental animals 63.3 (2014): 321-330, both of which are incorporated herein by reference in the entirety.

The genetically modified non-human animal can also be various other animals, e.g., a rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable ES cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiment, the rodent is selected from the superfamily Muroidea. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In one embodiment, the non-human animal is a mouse.

In some embodiments, the animal is a mouse of a strain selected from BALB/c, A, A/He, A/J, A/WySN, AKR, AKR/A, AKR/J, AKR/N, TA1, TA2, RF, SWR, C3H, C57BR, SJL, C57L, DBA/2, KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, C57BL/O1a, C57BL, C58, CBA/Br, CBA/Ca, CBA/J, CBA/st, and CBA/H. In some embodiments, the mouse is a 129 strain selected from the group consisting of a strain that is 129P1, 129P2, 129P3, 129X1, 129S1 (e.g., 129SI/SV, 129S1/SvIm), 129S2, 12954, 129S5, 129S9/SvEvH, 12956 (129/SvEvTac), 12957, 12958, 129T1, 129T2. These mice are described, e.g., in Festing et al., Revised nomenclature for strain 129 mice, Mammalian Genome 10:836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BU6 strains. In some embodiment, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the animal expressing human or humanized B2M and/or MHC molecule (e.g., MHC class I α chain) is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes).

Non-limiting examples of such mice include, e.g., NOD mice, SCID mice, NOD/SCID mice, nude mice, NOD/SCID nude mice, NOD-Rag 1^{-/-}-IL2rg^{-/-} (NRG) mice, Rag 2^{-/-}-IL2rg^{-/-} (RG) mice, B-NDG (NOD-Prkdc^{scid} IL-2rγ^{null}) mice, and Rag1 and/or Rag2 knockout mice. In some embodiments, these mice can optionally be irradiated, or otherwise treated to destroy one or more immune cell types. Thus, in various embodiments, a genetically modified mouse is provided that can include one or more mutations at the endogenous non-human B2M or MHC gene locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, B-NDG (NOD-Prkdc^{scid} IL-2rγ^{null}) mice, nude mice, Rag1 and/or Rag2 knockout mice, and a combination thereof. These genetically modified animals are described, e.g., in U.S. Ser. No. 10/820,580B2 and PCT/CN2018/079365; each of which is incorporated herein by reference in its entirety.

Although genetically modified cells are also provided that can comprise the modifications (e.g., disruption, mutations) described herein (e.g., ES cells, somatic cells), in many embodiments, the genetically modified non-human animals comprise the modification of the endogenous B2M and/or MHC gene locus in the germline of the animal.

Furthermore, the genetically modified animal can be homozygous with respect to the modifications (e.g., replacement) of the endogenous B2M and/or MHC gene. In some embodiments, the animal can be heterozygous with respect to the modification (e.g., replacement) of the endogenous B2M and/or MHC gene.

The present disclosure further relates to a non-human mammal generated through the methods as described herein. In some embodiments, the genome thereof contains human gene(s).

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains a modification (e.g., replacement) of the B2M and/or MHC gene in the genome of the animal.

In some embodiments, the expression of human or humanized MHC protein complex, human or humanized B2M, human or humanized MHC gene (e.g., MHC class I α chain), and/or the fusion protein in a genetically modified animal is controllable, as by the addition of a specific inducer or repressor substance. In some embodiments, the specific inducer is selected from Tet-Off System/Tet-On System, or Tamoxifen System.

In some embodiments, the genetically-modified, non-human animal comprising a humanized heavy chain immunoglobulin locus and/or a humanized light chain immunoglobulin locus. In some embodiments, the animal comprises one or more human IGHV genes, one or more human IGHD genes, one or more human IGHJ genes, one or more human IGKV genes and/or one or more human IGKJ genes. In some embodiments, these genes are at the endogenous immunoglobulin gene locus.

In some embodiments, the animal comprises a human lambda chain immunoglobulin locus. In some embodiments, the animal comprises a disruption in the animal's endogenous lambda light chain immunoglobulin gene locus. In some embodiments, the animal does not have a disruption in the animal's endogenous lambda light chain immunoglobulin gene locus.

The animal can have one or more other genetic modifications, and/or other modifications, that are suitable for the particular purpose for which the humanized animal is made.

Genetically modified non-human animals that comprise a modification of an endogenous non-human immunoglobulin gene locus. In some embodiments, the modification can comprise a human nucleic acid sequence encoding at least a portion of a human protein (e.g., at least 10%, 20%, 30%, 40%, 50%, 60%, 70%, 80%, 90%, 95%, 96%, 97%, 98%, or 99% identical to the human heavy chain variable domain or light chain variable domain sequence). Although genetically modified cells are also provided that can comprise the modifications described herein (e.g., ES cells, somatic cells), in many embodiments, the genetically modified non-human animals comprise the modification of the endogenous locus in the germline of the animal.

Genetically modified animals can express a humanized antibody and/or a chimeric antibody from endogenous mouse loci, wherein one or more endogenous mouse immunoglobulin genes have been replaced with human immunoglobulin genes and/or a nucleotide sequence that is at least 10%, 20%, 30%, 40%, 50%, 60%, 70%, 80%, 90%, 95%, 96%, 97%, 98%, or 99% identical to the human immunoglobulin gene sequences (e.g., IGHV, IGHD, IGHJ, IGKV and/or IGKJ genes). In various embodiments, an endogenous non-human immunoglobulin gene locus is modified in whole or in part to comprise human nucleic acid sequence.

Genetic, molecular and behavioral analyses for the non-human mammals described above can performed. The present disclosure also relates to the progeny produced by the non-human mammal provided by the present disclosure mated with the same or other genotypes. Non-human mammals can be any non-human animal known in the art and which can be used in the methods as described herein. Preferred non-human mammals are mammals, (e.g., rodents). In some embodiments, the non-human mammal is a mouse.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human or humanized immunoglobulins can be detected by a variety of methods.

There are many analytical methods that can be used to detect exogenous DNA or modifications on the genomic DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized proteins.

Genetic, molecular and behavioral analyses for the non-human mammals described above can be performed. The present disclosure also relates to the progeny produced by the non-human mammal provided by the present disclosure mated with the same or other genotypes.

In some embodiments, the genome of the genetically modified non-human animal described herein includes a sequence that is at least 80%, 85%, 90%, 91%, 92%, 93%, 94%, 95%, 96%, 97%, 98%, or at least 99% identical to any of SEQ ID NOs: 81, 82, 83, 84, 85, 86, 87, 88, 89, 90, and/or 91.

### Antibodies and Antigen Binding Fragments

The present disclosure provides antibodies and antigen-binding fragments thereof (e.g., humanized antibodies or chimeric antibodies) that are produced by the methods described herein.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or subclasses including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001.422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Boume, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgG4, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain.

In some embodiments, the scFV has one heavy chain variable domain, and one light chain variable domain. In some embodiments, the scFV has two heavy chain variable domains, and two light chain variable domains. In some embodiments, the scFV has two antigen binding regions, and the two antigen binding regions can bind to the respective target antigens.

The antibodies and antigen-binding fragments thereof (e.g., humanized antibodies or chimeric antibodies) that are produced by the methods described herein have various advantages. In some embodiments, no further optimization is required to obtain desired properties (e.g., binding affinities, thermal stabilities, and/or limited aggregation).

In some implementations, the antibody (or antigen-binding fragments thereof) specifically binds to a target with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1>10⁻⁸ M, less than 1×10⁻⁹ M, or less than 1×10⁻¹⁰ M. In some embodiments, the KD is less than 50 nM, 40 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. In some embodiments, the antibody binds to a target with KD less than or equal to about 0.9 nM, 0.8 nM, 0.7 nM, 0.6 nM, 0.5 nM, 0.4 nM, 0.3 nM, 0.2 nM, or 0.1 nM.

In some embodiments, thermal stabilities are determined. The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

As IgG can be described as a multi-domain protein, the melting curve sometimes shows two transitions, or three transitions, with a first denaturation temperature, Tm D1, and a second denaturation temperature Tm D2, and optionally a third denaturation temperature Tm D3.

In some embodiments, the antibodies or antigen binding fragments as described herein has a Tm D1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm D2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm D3 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, Tm, Tm D1, Tm D2, Tm D3 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies or antigen binding fragments as described herein do not form aggregation when the temperate is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies and antigen binding fragments thereof, e.g. TCR-like antibodies, specifically recognize a peptide epitope in the context of an MHC molecule, such as an MHC class I. In some cases, the MHC class I molecule is an HLA-A2 molecule, e.g. HLA-A2*01. In some embodiments, the MHC class I molecule is any one of the HLA molecules described herein, or any one of the fusion proteins described herein.

In some embodiments, the antibodies and antigen binding fragments thereof can specifically recognize a peptide epitope in an MHC molecule dependent manner. In some embodiments, the antibodies and antigen binding fragments thereof can specifically recognize a peptide epitope in an MHC molecule independent manner.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

In some embodiments, the genetically modified animals can be made by introducing human or humanized genes into the genome of non-human animals. In some embodiments, the methods first involve modifying the human immunoglobulin locus on the human chromosome. The modified human chromosomes are then introduced into the mouse recipient cell. The human immunoglobulin variable region is then introduced into the corresponding region of the mouse genome by direct replacement. Then, the recipient cells are screened. In some embodiments, the cells do not contain the human chromosomes. The cells are then injected to blastocysts to prepare chimeric mice. Subsequent breeding can be performed to obtain mice containing intact humanized immunoglobulin locus.

In some embodiments, the methods for making a genetically modified, humanized animal, can include the step of replacing at an endogenous locus (or site), a nucleic acid (e.g., V, D, J regions, or V, J regions) with a corresponding region of human sequence. The sequence can include a region (e.g., a part or the entire region) of IGHV, IGHD, IGHJ, IGKV, and/or IGKJ genes. In some embodiments, the replacement is mediated by homologous recombination. In some embodiments, the replacement is mediated by Cre recombinase.

In some embodiments, the disclosure provides replacing in at least one cell of the animal, at an endogenous B2M or MHC gene locus, a sequence encoding a region of an endogenous B2M or MHC α chain with a sequence encoding a fusion protein described herein. In some embodiments, the replacement occurs in a germ cell, a somatic cell, a blastocyst, or a fibroblast, etc. The nucleus of a somatic cell or the fibroblast can be inserted into an enucleated oocyte.

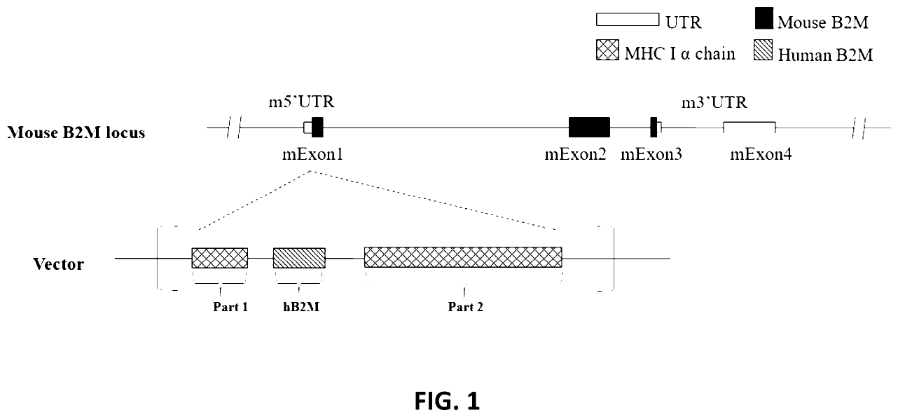

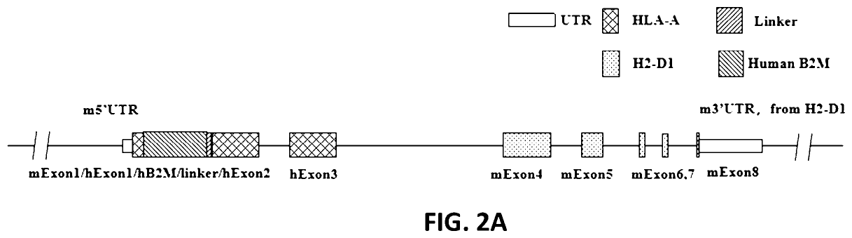

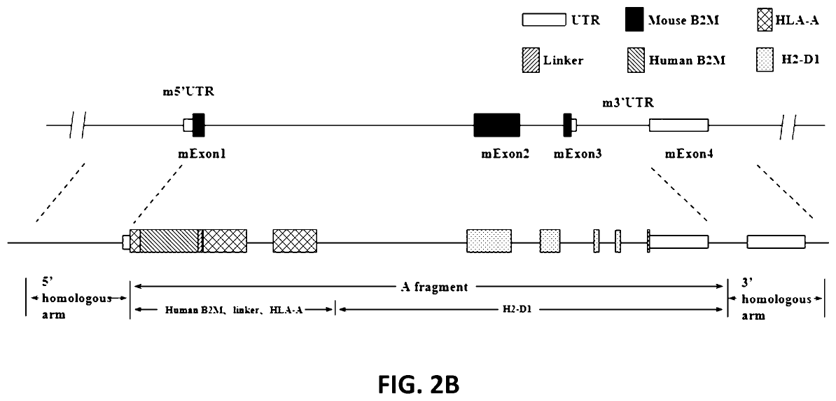

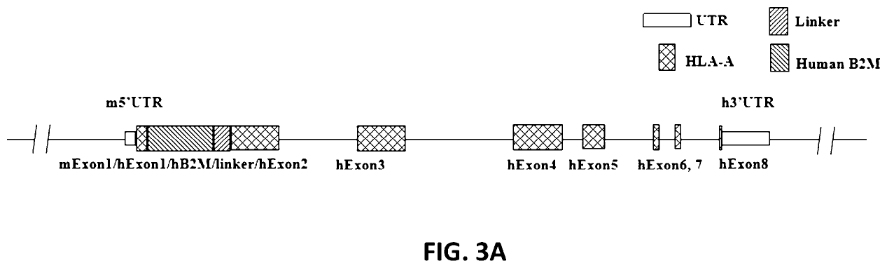

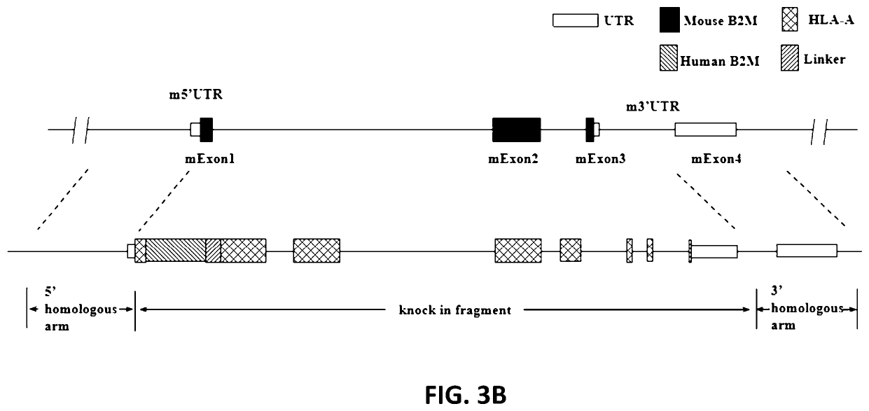

FIGS. 1, 2B and 3B show a MHC protein complex humanization strategy for at mouse B2M gene locus. In FIG. 2B and FIG. 3B, the targeting strategy involves a vector comprising the 5′ homologous arm, a sequence encoding the fusion protein, and the 3′ homologous arm. The process can involve replacing endogenous B2M gene sequence with the sequence encoding the fusion protein by homologous recombination. In some embodiments, the cleavage at the upstream and the downstream of the target site (e.g., by zinc finger nucleases, TALEN or CRISPR) can result in DNA double strands break, and the homologous recombination is used to replace endogenous B2M gene sequence with the sequence encoding the fusion protein.

In some embodiments, the targeting strategy in FIG. 2B can be used to generate a genetically modified non-human animal expressing a fusion protein (or a chimeric fusion protein). In some embodiments, the expressed fusion protein comprises, from N-terminus to C-terminus: (a) a signal peptide of a human HLA (e.g., any one of the HLA molecules described herein), (b) human B2M, (c) a linker peptide, (d) α1 and α2 domains of the human HLA, (e) α3 domain, connecting peptide, transmembrane region, and cytoplasmic region of mouse H2-D1. In some embodiments, the fusion protein comprises or consists of an amino acid sequence that is at least 80%, 85%, 90%, 91%, 92%, 93%, 94%, 95%, 96%, 97%, 98%, or at least 99% identical to SEQ ID NO: 61, 100, 101, 102, 103, 104, 105, 106, 107, 108, 109, 110, 111, 112, 113, 114, 115, 116, or 117.

In some embodiments, the targeting strategy in FIG. 3B can be used to generate a genetically modified non-human animal expressing a fusion protein. In some embodiments, the expressed fusion protein comprises, from N-terminus to C-terminus: (a) a signal peptide of a human HLA (e.g., any one of the HLA molecules described herein), (b) human B2M, (c) a linker peptide, and (d) α1 domain, α2 domain, α3 domain, connecting peptide, transmembrane region, and cytoplasmic region of the human HLA. In some embodiments, the fusion protein comprises or consists of an amino acid sequence that is at least 80%, 85%, 90%, 91%, 92%, 93%, 94%, 95%, 96%, 97%, 98%, or at least 99% identical to SEQ ID NO: 62, 63, 64, 65, 66, 67, 68, 69, 70, 71, 72, 73, 74, 75, 76, 77, 78, 79, or 80.

In some embodiments, the linker peptide described herein comprises at least 1, 2, 3, 4, 5, 6, 7, or 8 repeats of GGGGS (SEQ ID NO: 5). In some embodiments, the linker peptide is a flexible linker. Details of flexible linkers can be found, e.g., Chen, X., et al. “Fusion protein linkers: property, design and functionality.” Advanced Drug Delivery Reviews 65.10 (2013): 1357-1369, which is incorporated herein by reference in its entirety. In some embodiments, the linker peptide comprises or consists of an amino acid sequence that is at least 85%, 90%, 95%, 96%, 97%, 98%, 99%, or 100% identical to SEQ ID NO: 4.

In some embodiments, the sequence between the 5′ end targeting site and the 3′ end targeting site is knocked out. In some embodiments, the sequence between the 5′ end targeting site and the 3′ end targeting site is replaced. In some embodiments, the replaced sequence starts from within exon 1 or intron 1 of mouse B2M gene. In some embodiments, the replaced sequence ends within exon 3 or intron 3 of mouse B2M gene.

Thus, in some embodiments, the methods for making a genetically modified, humanized animal, can include the step of replacing at an endogenous B2M locus (or site), a nucleic acid encoding a sequence encoding a region of endogenous B2M with a sequence encoding the fusion protein described herein. The sequence can include a region (e.g., a part or the entire region) of exon 1, exon 2, exon 3, exon 4 of a an endogenous B2M gene. In some embodiments, the sequence encoding the fusion protein includes a region (e.g., a part or the entire region) of exon 1, exon 2, exon 3, exon 4 of a human B2M gene, and a region (e.g., a part or the entire region) of exon 1, exon 2, exon 3, exon 4, exon 5, exon 6, exon 7, and exon 8 of an endogenous or human MHC α chain gene. In some embodiments, the endogenous B2M locus is a portion of exon 1, exon 2, and a portion of exon 3 of mouse B2M gene.

In some embodiments, the methods of modifying a B2M gene locus of a mouse to express the fusion protein described herein can include the steps of replacing at the endogenous mouse B2M gene locus a nucleotide sequence encoding a mouse B2M with a nucleotide sequence encoding the fusion protein, thereby generating a sequence encoding a fusion protein comprising a human B2M and a human or chimeric MHC α chain.

In some embodiments, the nucleotide sequences as described herein do not overlap with each other (e.g., the 5′ homologous arm, the A fragment (or the BNDG-A fragment), and/or the 3′ homologous arm do not overlap). In some embodiments, the amino acid sequences as described herein do not overlap with each other.

Zinc finger proteins, TAL-effector domains, or single guide RNA (sgRNA) DNA-binding domains can be designed to target regions within exon 1, exon 2, exon 3, exon 4, intron 1, intron 2, and/or intron 3 of endogenous (e.g., mouse) B2M gene locus. After the zinc finger proteins, TAL-effector domains, or single guide RNA (sgRNA) DNA-binding domains bind to the target sequences, the nuclease cleaves the genomic DNA. In some embodiments, the nuclease is CRISPR associated protein 9 (Cas9).

Thus, the methods of producing a mouse expressing a human or humanized MHC protein complex, human or humanized B2M, and/or human or humanized MHC molecules can involve one or more of the following steps: transforming a mouse embryonic stem cell with a gene editing system that targets endogenous B2M or MHC gene, thereby producing a transformed embryonic stem cell; introducing the transformed embryonic stem cell into a mouse blastocyst; implanting the mouse blastocyst into a pseudopregnant female mouse; and allowing the blastocyst to undergo fetal development to term.

In some embodiments, the transformed embryonic cell is directly implanted into a pseudopregnant female mouse instead, and the embryonic cell undergoes fetal development.

In some embodiments, the gene editing system can involve Zinc finger proteins, TAL-effector domains, or single guide RNA (sgRNA) DNA-binding domains.

The present disclosure further provides a method for establishing an animal model expressing a human or humanized MHC protein complex, human or humanized B2M, and/or human or humanized MHC molecules, involving the following steps:
(a) providing the cell (e.g. a fertilized egg cell) with the genetic modification based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a RenMab mouse, a C57BL/6 mouse, a NOD/scid mouse, a NOD/scid nude mouse, or a B-NDG mouse). In some embodiments, the non-human mammal is a B-NDG (NOD-Prkdc^{scid} IL-2rγ^{null}) mouse. In some embodiments, the non-human mammal is a NOD/scid mouse.

In some embodiments, the fertilized eggs for the methods described above are RenMab™ mouse fertilized egges, RenLite™ mouse fertilized eggs, NOD/scid fertilized eggs, NOD/scid nude fertilized eggs, or B-NDG fertilized eggs. Other fertilized eggs that can also be used in the methods as described herein include, but are not limited to, C57BL/6 fertilized eggs, FVB/N fertilized eggs, BALB/c fertilized eggs, DBA/l fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the method described above.

The present disclosure further provides a method for establishing a humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57 mouse, a BALB/c mouse, or a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/l fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

Cells, tissues, and animals (e.g., mouse) are also provided that comprise the nucleotide sequences as described herein, as well as cells, tissues, and animals (e.g., mouse) that express humanized or chimeric antibodies from an endogenous non-human locus.

The present disclosure further relates to methods for generating genetically modified animal model with two or more human or chimeric genes. The animal can comprise one or more human or humanized immunoglobulin locus and a sequence encoding an additional human or chimeric protein. In some embodiments, the additional human or chimeric protein can be programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), or TNF Receptor Superfamily Member 4 (TNFRSF4 or OX40).

The methods of generating genetically modified animal model with additional human or chimeric genes (e.g., humanized genes) can include the following steps:
(a) using the methods as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric PD-1, CTLA-4, LAG-3, BTLA, PD-L1, CD27, CD28, CD47, CD137, CD154, TIGIT, TIM-3, GITR, SIRPa, or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

### Methods of Using Genetic Modified Animals

The genetic modified animals can be used to generate humanized or chimeric antibodies that can bind specifically to a target. In some embodiments, the target (e.g., a protein or a fragment of the protein) can be used as an immunogen to generate antibodies in these animals using standard techniques for polyclonal and monoclonal antibody preparation. In some embodiments, the genetic modified animal is exposed to a selected antigen for a time and under conditions which permit the animal to produce antibody specific for the antigen.

Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence and encompasses an epitope of the protein such that an antibody raised against the peptide forms a specific immune complex with the protein.

An immunogen typically is used to prepare antibodies by immunizing a suitable subject (e.g., the genetically modified animal as described herein). An appropriate immunogenic preparation can contain, for example, a recombinantly-expressed or a chemically-synthesized polypeptide (e.g., a fragment of the protein). The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

In one aspect, the disclosure provides a mouse that comprises a modification of an endogenous immunoglobulin heavy chain locus, wherein the mouse produces a B cell that comprises a rearranged immunoglobulin sequence operably linked to a heavy chain constant region gene sequence. In some embodiment, the rearranged immunoglobulin sequence operably linked to the heavy chain constant region gene sequence comprises a human heavy chain V, D, and/or J sequence. In some embodiments, the heavy chain constant region gene sequence comprises a human or a mouse heavy chain sequence selected from the group consisting of a CH1, a hinge, a CH2, a CH3, and a combination thereof.

In one aspect, the disclosure provides a mouse that comprises a modification of an endogenous immunoglobulin light chain (e.g., kappa or lambda) locus, wherein the mouse produces a B cell that comprises a rearranged immunoglobulin sequence operably linked to a light chain constant region gene sequence. In some embodiments, the rearranged immunoglobulin sequence operably linked to the light chain constant region gene sequence comprises a human light chain V and/or J sequence. In some embodiments, the light chain constant region gene sequence comprises a human or a mouse light chain constant region.

The mouse B cells or spleen cells can comprise a rearranged non-mouse immunoglobulin variable gene sequence, e.g., operably linked to a mouse immunoglobulin constant region gene. The sequences for encoding human heavy chain variable region and human light chain variable region are determined. The sequences can be determined by e.g., sequencing the hybridoma of interest or B cells. In some embodiments, single B cell screening is used. It can screen the natural antibody repertoire without the need for hybridoma fusion and combinatorial display. For example, B cells can be mixed with a panel of DNA-barcoded antigens, such that both the antigen barcode(s) and B-cell receptor (BCR) sequences of individual B cells are recovered via single-cell sequencing protocols.

The antibodies can be further modified to obtain a humanized antibody or a human antibody, e.g., by operably linking the sequence encoding human heavy chain variable region to a sequence encoding a human heavy chain constant region, and/or operably linking the sequence encoding human light chain variable region to a sequence encoding a human light chain constant region.

In some embodiments, if the mouse expresses a protein that is very similar to the antigen of interest, it can be difficult to elicit an immune response in the mouse. This is because during immune cell development, B-cells and T-cells that recognize MHC molecules bound to peptides of self-origin are deleted from the repertoire of immune cells. In those cases, the humanized mouse can be further modified. The corresponding gene in the mouse can be knocked out, and the mouse is then exposed to the antigen of interest. Because the mouse does not go through negative selection for the gene product, the mouse can generate an antibody that can specifically bind to the target easily.

The disclosure also provides methods of making antibodies, nucleic acids, cells, tissues (e.g., spleen tissue). In some embodiments, the methods involve exposing the animal as described herein to the antigen. Antibodies (e.g., hybrid antibodies), nucleic acids encoding the antibodies, cells, and/or tissues (e.g., spleen tissue) can be obtained from the animal. In some embodiments, the nucleic acids encoding human heavy and light chain immunoglobulin variable regions are determined, e.g., by sequencing. In some embodiments, the nucleic acid encoding the human heavy chain immunoglobulin variable region can be operably linked with a nucleic acid encoding a human heavy chain immunoglobulin constant region. In some embodiments, the nucleic acid encoding the human light chain immunoglobulin variable region can be operably linked with a nucleic acid encoding a human light chain immunoglobulin constant region. In some embodiments, the cells containing the nucleic acids as described herein are cultured and the antibodies are collected.

In some embodiments, no mouse immunoglobulin V, D, J genes (e.g., no mouse IGHV, IGHD, IGHJ, IGKV, or IGKJ genes) contributes to the heavy chain and/or light chain variable region sequence. In some embodiments, the heavy chain and/or light chain variable region sequence produced by the animal are fully human, and are completely contributed by human immunoglobulin V, D, J genes (e.g., human IGHV, IGHD, IGHJ, IGKV, and IGKJ genes).

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

Additional modifications to the antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In one aspect, the MHC-I VH/VL mice described herein can be immunized with one or more antigen-MHC complexes. For specific antigens, MHC-tolerant mice for the corresponding antigens can be selected for immunization (i.e., mice expressing the corresponding HLA). Antigen peptides that can be used for immunization are shown in the table below. In some embodiment, the antigen of the antigen-MHC complex described herein include a peptide comprising an amino acid sequence as set forth in SEQ ID NOs: 23-55.

TABLE 16

| Antigen | MHC haplotype | Sequence | SEQ ID NO |
|---|---|---|---|
| AFP | HLA-A*0201 | FMNKFIYEI | 23 |
| gp100 | HLA-A*0201 | KTWGQYWQV | 24 |
| gp100 | HLA-A*0201 | IMDQVPFSV | 25 |
| gp100 | HLA-A*0201 | YLEPGPVTV/A | 26 |
| gp100 | HLA-A*0201 | IMDQVPFSV | 27 |
| gp100 | HLA-A*0201 | ITDQVPFSV | 28 |
| HA-1H | HLA-A*0201 | VLHDDLLEA | 29 |
| hCGβ | HLA-A*0201 | GVLPALPQV | 30 |
| hCGβ | HLA-A*0201 | TMTRVLQGC | 31 |
| Her/Neu | HLA-A*0201 | KIFGSLAFL | 32 |
| kras G12D | HLA-C*0802 | GADGVGKSA | 33 |
| kras G12V | HLA-A*03 | VVVGAVGVGK | 34 |
| kras Q61H | HLA-A*01 | ILDTAGHEEY | 35 |
| MAGE3 | HLA-A*0201 | FLWGPRALV | 36 |
| MAGEA1 | HLA-A*0101 | EADPTGHSY | 37 |
| MART-1 | HLA-A*0201 | AAGIGILTV | 38 |
| MIF | HLA-A*0201 | FLSELTQQL | 39 |
| MUC1 | HLA-A*0201 | LLLTVLTW | 40 |
| NPM1c | HLA-A*0201 | AIQDLCLAV | 41 |
| NY-ESO-1 | HLA-A*0201 | SLLMWITQC | 42 |
| p53 | HLA-A*0201 | LLGRNSFEV | 43 |
| p53 | HLA-A*0201 | RMPEAAPPV | 44 |
| p53 | HLA-A*0201 | GLAPPQHLIRV | 45 |
| p53 R175H | HLA-A*0201 | HMTEVVRHC | 46 |
| p53 Y220C | HLA-A*0201 | VVPCEPPEV | 47 |
| p68 | HLA-A*0201 | YLLPAIVHI | 48 |
| PR1 | HLA-A*0201 | VLQELNVTV | 49 |
| PRAME | HLA-A*0201 | ALYVDSLFFL | 50 |
| PRAME | HLA-A*0201 | ALYVDSLFFL | 51 |
| TARP | HLA-A*0201 | FLRNFSLML | 52 |
| TERT | HLA-A*0201 | ILAKFLHWL | 53 |
| TERT | HLA-A*0201 | RLVDDFLLV | 54 |
| WT1 | HLA-A*0201 | RMFPNAPYL | 55 |

In some embodiments, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, or at least 60% antibodies generated in any of the genetically-modified non-human animal comprises a VH CDR3 having an amino acid sequence that is longer than 15, 16, 17, 18, 19, or 20 amino acids.

The antibody or antigen-binding portion thereof can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against a peptide in the context of an MHC molecule can also be referred to as a TCR-like CAR. Thus, among the provided binding molecules are antigen receptors, such as those that include one of the provided antibodies, e.g., TCR-like antibodies. In some embodiments, the antigen receptors and other chimeric receptors specifically bind to a region or epitope, e.g. TCR-like antibodies. Among the antigen receptors are functional non-TCR antigen receptors, such as chimeric antigen receptors (CARs). Also provided are cells expressing the CARs and uses thereof in adoptive cell therapy, such as treatment of diseases and disorders associated with antigen expression.

TCR-like CARs that contain a non-TCR molecule that exhibits T cell receptor specificity, such as for a T cell epitope or peptide epitope when displayed or presented in the context of an MHC molecule. In some embodiments, a TCR-like CAR can contain an antibody or antigen-binding portion thereof, e.g., TCR-like antibody, such as described herein. In some embodiments, the antibody or antibody-binding portion thereof is reactive against specific peptide epitope in the context of an MHC molecule, wherein the antibody or antibody fragment can differentiate the specific peptide in the context of the MHC molecule from the MHC molecule alone, the specific peptide alone, and, in some cases, an irrelevant peptide in the context of an MHC molecule. In some embodiments, an antibody or antigen-binding portion thereof can exhibit a higher binding affinity than a T cell receptor.

In some embodiments, the antibody or antigen-binding portion thereof can be expressed on cells as part of a T cell receptor.

The present disclosure also provides methods of determining an antibody that can specifically binds to an antigen peptide-MHC complex. The methods involve incubating the antibodies with cells presenting the antigen peptide of interest, cells presenting a control antigen peptide and/or cells do not present any antigen peptide; and determining that the antibodies can specifically bind to cells presenting the antigen peptide of interest, and optionally determining that the antibodies cannot bind to the cells presenting the control antigen peptide or the cells that do not present any antigen peptide. The MHC can be any MHC as descried herein.

The present disclosure also provides methods of determining VH and VL sequences for an antibody that can specifically binds to an antigen of interest (e.g., an peptide-MHC complex). In some embodiments, the methods involve exposing the animal as described herein to the antigen of interest (e.g., a peptide-MHC complex comprising an antigen peptide of interest); and determining a cell from a tissue of the animal (e.g., a spleen tissue, a lymphoid tissue) that expresses an antibody that binds to the antigen of interest; and sequencing nucleic acids encoding human heavy and light chain immunoglobulin variable regions in the cell that expresses the antibody. In some embodiments, the sequences are determined by Beacon® system.

The present disclosure provides a genetically engineered animal that expresses a limited repertoire of light chains that can be associated with a diversity of heavy chains. In various embodiments, the endogenous kappa light chain variable region genes are deleted and replaced with a single, two, three, four, or five human light chain variable region genes, operably linked to the endogenous kappa constant region gene. In various embodiments, the animal also comprises a nonfunctional lambda light chain locus, or a deletion thereof or a deletion that renders the locus unable to make a lambda light chain.

In some embodiments, the animal comprises a light chain variable region locus lacking an endogenous light chain variable gene and comprising a rearranged human V/J sequence, operably linked to an endogenous constant region, and wherein the locus expresses a light chain comprising the human V/J sequence linked to the endogenous constant region.

In some embodiments, the methods described here are designed to make a bispecific antibody, e.g., a bispecific antibody that binds to two antigen-MHC complexes.

In some embodiments, the animal comprises a rearranged light chain variable region locus. In some embodiments, the bispecific antibody can have a common light chain. In some embodiments, the methods involve immunizing a first animal comprising a rearranged light chain variable region locus, and obtaining the VH and the VL sequence for the antibody. Then the methods involve immunizing a second animal comprising the rearranged light chain variable region locus, and obtaining the VH and the VL sequence for the antibody.

Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, knob-into-hole (KIH) technology can be used, which involves engineering CH3 domains to create either a “knob” or a “hole” in each heavy chain to promote heterodimerization. The KIH technique is described e.g., in Xu, Yiren, et al. “Production of bispecific antibodies in ‘knobs-into-holes’ using a cell-free expression system.” MAbs. Vol. 7. No. 1. Taylor & Francis, 2015, which is incorporated by reference in its entirety. In some embodiments, one heavy chain has a T366W, and/or S354C (knob) substitution (EU numbering), and the other heavy chain has an Y349C, T366S, L368A, and/or Y407V (hole) substitution (EU numbering). In some embodiments, one heavy chain has one or more of the following substitutions Y349C and T366W (EU numbering). The other heavy chain can have one or more the following substitutions E356C, T366S, L368A, and Y407V (EU numbering). In some embodiments, one heavy chain has a T366Y (knob) substitution, and the other heavy chain has one, two, or three of these substitutions T366S, L368A, Y407V (hole).

### EXAMPLES

The invention is further described in the following examples, which do not limit the scope of the invention described in the claims.

### Example 1: Generation of MHC Humanized Mice

Nucleotide sequences encoding human B2M and HLA Class I proteins were introduced into non-human animals comprising rearranged or unrearranged human or humanized immunoglobulin heavy and/or light chain loci, to obtain genetically-modified non-human animals (MHC-I VH/VL background) that can express and tolerate human MHC protein molecules in vivo. The non-human animals can be RenMab™ mice (hVH/hVL mice), which have both a humanized immunoglobulin heavy chain locus and a humanized immunoglobulin kappa chain locus. Detailed descriptions of RenMab™ mice can be found, e.g., in PCT/CN2020/075698 and US20200390073A1, which are incorporated herein by reference in the entirety. Alternatively, the non-human animals can be RenLite™ mice (or hVH/hcVL mice), which have a humanized immunoglobulin heavy chain locus and a humanized common light chain locus. Detailed descriptions of RenLite™ mice can be found, e.g., in PCT/CN2021/097652, which is incorporated herein by reference in its entirety. One or more gene editings can be performed directly in non-human animals containing human immunoglobulin sequences, to introduce gene sequences that can express human B2M and MHC class I proteins. Such animals can also be obtained by breeding non-human animals containing human immunoglobulin sequences with MHC humanized non-human animals. As shown in FIG. 1, non-limiting MHC humanization strategies include insertion of artificial MHC Class I genes encoding a B2M and MHC class I α chain fusion protein into the endogenous B2M locus. Optionally, the coding sequence for endogenous B2M can also be inactivated (e.g., knocked out). A specific scheme of MHC humanization can be found, e.g., in PCT/CN2021/070967, which is incorporated by reference herein in its entirety.

The targeting vector shown in FIG. 1 can include the following main features, in a 5′ to 3′ order: a homologous arm sequence upstream (SEQ ID NO: 1) of the insertion site, MHC class I α chain part 1 sequence, human B2M coding sequence, MHC class I α chain part 2 sequence, and a homologous arm sequence downstream (SEQ ID NO: 2) of the insertion site. Specifically, the MHC class I α chain part 1 sequence can include a sequence encoding the signal peptide sequence of human MHC class I α chain; the MHC class I α chain part 2 sequence can include sequences other than the sequence encoding the signal peptide, e.g., the coding sequence of the MHC class I α chain (e.g., a sequence encoding α1, α2, α3, transmembrane, and intracellular domains of human HLA protein; or a sequence encoding α1 and α2 domains of human HLA protein followed by a sequence encoding α3, transmembrane, and intracellular domains of mouse H2-D1 protein). Optionally, a linker sequence can also be inserted between the human B2M coding sequence and the MHC class I α chain part 2 sequence, e.g., a flexible linker sequence of 5′-GGAGGTGGCGGATCCGGCGGAGGCGGCTCGGGTGGCGGCGGCTCT-3′ (SEQ ID NO: 3), which encodes an amino acid sequence of (GGGGS)₃ (SEQ ID NO: 4). In order to facilitate expression of the fusion protein, a MHC class I α chain part 3 sequence can also be included downstream of the MHC class I α chain part 2 sequence. The MHC class I α chain part 3 sequence may include the 3′ UTR sequence of the MHC class I α chain gene and the downstream part of the regulatory sequence (e.g., 3′ UTR sequence and the downstream part of the regulatory sequence of human HLA gene or mouse H2-D1 gene).

In view of the high polymorphism of human HLA class I gene sequences, the large number of different allele genes, as well as their similar structures and functions, the targeting strategy described herein can be applied to all HLA class I genes. Some HLA gene information are shown in the table below. For example, mouse B32M locus can be modified by inserting sequences from human HLA-A gene (NCBI Gene ID: 3105, Primary source: HGNC: 4931, UniProt ID: β04439), to obtain a humanized mouse B2M gene locus shown in FIG. 2A or FIG. 3A. The corresponding targeting strategies are shown in FIG. 2B and FIG. 3B, respectively. The modified mice can express MHC complexes containing human B2M and human or humanized HLA-A.

TABLE 17

| | IMGT/HLA | | SEQ |
|---|---|---|---|
| HLA Gene | Acc No | Amino acid sequence | ID NO |
| HLA-A*0101 | HLA00001 | MAVMAPRTLLLLLSGALALTQTWAGSHSMRYFFTSVSRPGRGE | 58 |
| | | PRFIAVGYVDDTQFVRFDSDAASQKMEPRAPWIEQEGPEYWD | |
| | | QETRNMKAHSQTDRANLGTLRGYYNQSEDGSHTIQIMYGCDV | |
| | | GPDGRFLRGYRQDAYDGKDYIALNEDLRSWTAADMAAQITKR | |
| | | KWEAVHAAEQRRVYLEGRCVDGLRRYLENGKETLQRTDPPKTH | |
| | | MTHHPISDHEATLRCWALGFYPAEITLTWORDGEDQTQDTELV | |
| | | ETRPAGDGTFQKWAAVVVPSGEEQRYTCHVQHEGLPKPLTLR | |
| | | WELSSQPTIPIVGIIAGLVLLGAVITGAVVAAVMWRRKSSDRKG | |
| | | GSYTQAASSDSAQGSDVSLTACKV | |
| HLA-A*0201 | HLA00005 | MAVMAPRTLVLLLSGALALTQTWAGSHSMRYFFTSVSRPGRG | 6 |
| | | EPRFIAVGYVDDTQFVRFDSDAASQRMEPRAPWIEQEGPEYW | |
| | | DGETRKVKAHSQTHRVDLGTLRGYYNQSEAGSHTVQRMYGCD | |
| | | VGSDWRFLRGYHQYAYDGKDYIALKEDLRSWTAADMAAQTTK | |
| | | HKWEAAHVAEQLRAYLEGTCVEWLRRYLENGKETLQRTDAPKT | |
| | | HMTHHAVSDHEATLRCWALSFYPAEITLTWORDGEDQTQDTE | |
| | | LVETRPAGDGTFQKWAAVVVPSGQEQRYTCHVQHEGLPKPLT | |
| | | LRWEPSSQPTIPIVGIIAGLVLFGAVITGAVVAAVMWRRKSSDR | |
| | | KGGSYSQAASSDSAQGSDVSLTACKV | |
| HLA-A*0301 | HLA00101 | MAVMAPRTLLLLLSGALALTQTWAGSHSMRYFFTSVSRPGRGE | 7 |
| | | PRFIAVGYVDDTQFVRFDSDAASQRMEPRAPWIEQEGPEYWD | |
| | | QETRNVKAQSQTDRVDLGTLRGYYNQSEAGSHTIQIMYGCDV | |
| | | GSDGRFLRGYRQDAYDGKDYIALNEDLRSWTAADMAAQITKR | |
| | | KWEAAHEAEQLRAYLDGTCVEWLRRYLENGKETLQRTDPPKTH | |
| | | MTHHPISDHEATLRCWALGFYPAEITLTWORDGEDQTQDTELV | |
| | | ETRPAGDGTFQKWAAVVVPSGEEQRYTCHVQHEGLPKPLTLR | |
| | | WELSSQPTIPIVGIIAGLVLLGAVITGAVVAAVMWRRKSSDRKG | |
| | | GSYTQAASSDSAQGSDVSLTACKV | |
| HLA-A*0302 | HLA00040 | MAVMAPRTLLLLLSGALALTQTWAGSHSMRYFFTSVSRPGRGE | 8 |
| | | PRFIAVGYVDDTQFVRFDSDAASQRMEPRAPWIEQEGPEYWD | |
| | | QETRNVKAQSQTDRVDLGTLRGYYNQSEAGSHTIQIMYGCDV | |
| | | GSDGRFLRGYRQDAYDGKDYIALNEDLRSWTAADMAAQITKR | |
| | | KWEAAHVAEQQRAYLDGTCVEWLRRYLENGKETLQRTDPPKT | |
| | | HMTHHPISDHEATLRCWALGFYPAEITLTWORDGEDQTQDTEL | |
| | | VETRPAGDGTFQKWAAVVVPSGEEQRYTCHVQHEGLPKPLTLR | |
| | | WELSSQPTIPIVGIIAGLVLLGAVITGAVVAAVMWRRKSSDRKG | |
| | | GSYTQAASSDSAQGSDVSLTACKV | |
| HLA-A*1101 | HLA00043 | MAVMAPRTLLLLLSGALALTQTWAGSHSMRYFYTSVSRPGRGE | 9 |
| | | PRFIAVGYVDDTQFVRFDSDAASQRMEPRAPWIEQEGPEYWD | |
| | | QETRNVKAQSQTDRVDLGTLRGYYNQSEDGSHTIQIMYGCDV | |
| | | GPDGRFLRGYRQDAYDGKDYIALNEDLRSWTAADMAAQITKR | |
| | | KWEAAHAAEQQRAYLEGRCVEWLRRYLENGKETLQRTDPPKT | |
| | | HMTHHPISDHEATLRCWALGFYPAEITLTWORDGEDQTQDTEL | |
| | | VETRPAGDGTFQKWAAVVVPSGEEQRYTCHVQHEGLPKPLTLR | |
| | | WELSSQPTIPIVGIIAGLVLLGAVITGAVVAAVMWRRKSSDRKG | |
| | | GSYTQAASSDSAQGSDVSLTACKV | |
| HLA-A*2402 | HLA00050 | MAVMAPRTLVLLLSGALALTQTWAGSHSMRYFSTSVSRPGRG | 10 |
| | | EPRFIAVGYVDDTQFVRFDSDAASQRMEPRAPWIEQEGPEYW | |
| | | DEETGKVKAHSQTDRENLRIALRYYNQSEAGSHTLQMMFGCD | |
| | | VGSDGRFLRGYHQYAYDGKDYIALKEDLRSWTAADMAAQITKR | |
| | | KWEAAHVAEQQRAYLEGTCVDGLRRYLENGKETLQRTDPPKTH | |
| | | MTHHPISDHEATLRCWALGFYPAEITLTWORDGEDQTQDTELV | |
| | | ETRPAGDGTFQKWAAVVVPSGEEQRYTCHVQHEGLPKPLTLR | |
| | | WEPSSQPTVPIVGIIAGLVLLGAVITGAVVAAVMWRRNSSDRK | |
| | | GGSYSQAASSDSAQGSDVSLTACKV | |
| HLA-A*2901 | HLA00085 | MAVMAPRTLLLLLLGALALTQTWAGSHSMRYFTTSVSRPGRGE | 11 |
| | | PRFIAVGYVDDTQFVRFDSDAASQRMEPRAPWIEQEGPEYWD | |
| | | LQTRNVKAQSQTDRANLGTLRGYYNQSEAGSHTIQMMYGCH | |
| | | VGSDGRFLRGYRQDAYDGKDYIALNEDLRSWTAADMAAQITQ | |
| | | RKWEAARVAEQLRAYLEGTCVEWLRRYLENGKETLQRTDAPKT | |
| | | HMTHHAVSDHEATLRCWALSFYPAEITLTWQRDGEDQTQDTE | |
| | | LVETRPAGDGTFQKWASVVVPSGQEQRYTCHVQHEGLPKPLTL | |
| | | RWEPSSQPTIPIVGIIAGLVLFGAVFAGAVVAAVRWRRKSSDRK | |
| | | GGSYSQAASSDSAQGSDMSLTACKV | |
| HLA-A*3101 | HLA00097 | MAVMAPRTLLLLLLGALALTQTWAGSHSMRYFTTSVSRPGRGE | 12 |
| | | PRFIAVGYVDDTQFVRFDSDAASQRMEPRAPWIEQERPEYWD | |
| | | QETRNVKAHSQIDRVDLGTLRGYYNQSEAGSHTIQMMYGCDV | |
| | | GSDGRFLRGYQQDAYDGKDYIALNEDLRSWTAADMAAQITQR | |
| | | KWEAARVAEQLRAYLEGTCVEWLRRYLENGKETLQRTDPPKTH | |
| | | MTHHAVSDHEATLRCWALSFYPAEITLTWORDGEDQTQDTEL | |
| | | VETRPAGDGTFQKWASVVVPSGQEQRYTCHVQHEGLPKPLTL | |
| | | RWEPSSQPTIPIVGIIAGLVLFGAVFAGAVVAAVRWRRKSSDRK | |
| | | GGSYSQAASSDSAQGSDMSLTACKV | |
| HLA-A*3201 | HLA00101 | MAVMAPRTLLLLLLGALALTQTWAGSHSMRYFFTSVSRPGRGE | 13 |
| | | PRFIAVGYVDDTQFVRFDSDAASQRMEPRAPWIEQEGPEYWD | |
| | | QETRNVKAHSQTDRESLRIALRYYNQSEAGSHTIQMMYGCDV | |
| | | GPDGRLLRGYQQDAYDGKDYIALNEDLRSWTAADMAAQITQR | |
| | | KWEAARVAEQLRAYLEGTCVEWLRRYLENGKETLQRTDAPKTH | |
| | | MTHHAVSDHEATLRCWALSFYPAEITLTWORDGEDQTQDTEL | |
| | | VETRPAGDGTFQKWASVVVPSGQEQRYTCHVQHEGLPKPLTL | |
| | | RWEPSSQPTIPIVGIIAGLVLFGAMFAGAVVAAVRWRRKSSDRK | |
| | | GGSYSQAASSDSAQGSDMSLTACKV | |
| HLA-A*3301 | HLA00104 | MAVMAPRTLLLLLLGALALTQTWAGSHSMRYFTTSVSRPGRGE | 14 |
| | | PRFIAVGYVDDTQFVRFDSDAASQRMEPRAPWIEQEGPEYWD | |
| | | RNTRNVKAHSQIDRVDLGTLRGYYNQSEAGSHTIQMMYGCDV | |
| | | GSDGRFLRGYQQDAYDGKDYIALNEDLRSWTAADMAAQITQR | |
| | | KWEAARVAEQLRAYLEGTCVEWLRRHLENGKETLQRTDPPRTH | |
| | | MTHHAVSDHEATLRCWALSFYPAEITLTWORDGEDQTQDTEL | |
| | | VETRPAGDGTFQKWASVVVPSGQEQRYTCHVQHEGLPKPLTL | |
| | | RWEPSSQPTIPIVGIIAGLVLFGAVFAGAVVAAVRWRRKSSDRK | |
| | | GGSYSQAASSDSAQGSDMSLTACKV | |
| HLA-C*0802 | HLA00446 | MRVMAPRTLILLLSGALALTETWACSHSMRYFYTAVSRPGRGE | 15 |
| | | PRFIAVGYVDDTQFVQFDSDAASPRGEPRAPWVEQEGPEYWD | |
| | | RETQKYKRQAQTDRVSLRNLRGYYNQSEAGSHTLQRMYGCDL | |
| | | GPDGRLLRGYNQFAYDGKDYIALNEDLRSWTAADKAAQITQRK | |
| | | WEAAREAEQRRAYLEGTCVEWLRRYLENGKKTLQRAEHPKTHV | |
| | | THHPVSDHEATLRCWALGFYPAEITLTWORDGEDQTQDTELVE | |
| | | TRPAGDGTFQKWAAVVVPSGEEQRYTCHVQHEGLPEPLTLRW | |
| | | GPSSQPTIPIVGIVAGLAVLAVLAVLGAVMAVVMCRRKSSGGK | |
| | | GGSCSQAASSNSAQGSDESLIACKA | |
| HLA-A*3303 | HLA00106 | MAVMAPRTLLLLLLGALALTQTWAGSHSMRYFTTSVSRPGRGE | 92 |
| | | PRFIAVGYVDDTQFVRFDSDAASQRMEPRAPWIEQEGPEYWD | |
| | | RNTRNVKAHSQIDRVDLGTLRGYYNQSEAGSHTIQMMYGCDV | |
| | | GSDGRFLRGYQQDAYDGKDYIALNEDLRSWTAADMAAQITQR | |
| | | KWEAARVAEQLRAYLEGTCVEWLRRYLENGKETLQRTDPPKTH | |
| | | MTHHAVSDHEATLRCWALSFYPAEITLTWQRDGEDQTQDTEL | |
| | | VETRPAGDGTFQKWASVVVPSGQEQRYTCHVQHEGLPKPLTL | |
| | | RWEPSSQPTIPIVGIIAGLVLFGAVFAGAVVAAVRWRRKSSDRK | |
| | | GGSYSQAASSDSAQGSDMSLTACKV | |
| HLA-B*4402 | HLA00318 | MRVTAPRTLLLLLWGAVALTETWAGSHSMRYFYTAMSRPGRG | 93 |
| | | EPRFITVGYVDDTLFVRFDSDATSPRKEPRAPWIEQEGPEYWDR | |
| | | ETQISKTNTQTYRENLRTALRYYNQSEAGSHIIQRMYGCDVGPD | |
| | | GRLLRGYDQDAYDGKDYIALNEDLSSWTAADTAAQITQRKWE | |
| | | AARVAEQDRAYLEGLCVESLRRYLENGKETLQRADPPKTHVTHH | |
| | | PISDHEVTLRCWALGFYPAEITLTWORDGEDQTQDTELVETRPA | |
| | | GDRTFQKWAAVVVPSGEEQRYTCHVQHEGLPKPLTLRWEPSS | |
| | | QSTVPIVGIVAGLAVLAVVVIGAVVAAVMCRRKSSGGKGGSYS | |
| | | QAACSDSAQGSDVSLTA | |
| HLA-B*0702 | HLA00132 | MLVMAPRTVLLLLSAALALTETWAGSHSMRYFYTSVSRPGRGE | 94 |
| | | PRFISVGYVDDTQFVRFDSDAASPREEPRAPWIEQEGPEYWDR | |
| | | NTQIYKAQAQTDRESLRNLRGYYNQSEAGSHTLQSMYGCDVG | |
| | | PDGRLLRGHDQYAYDGKDYIALNEDLRSWTAADTAAQITQRK | |
| | | WEAAREAEQRRAYLEGECVEWLRRYLENGKDKLERADPPKTHV | |
| | | THHPISDHEATLRCWALGFYPAEITLTWORDGEDQTQDTELVE | |
| | | TRPAGDRTFQKWAAVVVPSGEEQRYTCHVQHEGLPKPLTLRW | |
| | | EPSSQSTVPIVGIVAGLAVLAVVVIGAVVAAVMCRRKSSGGKGG | |
| | | SYSQAACSDSAQGSDVSLTA | |
| HLA-C*0702 | HLA00434 | MRVMAPRALLLLLSGGLALTETWACSHSMRYFDTAVSRPGRGE | 95 |
| | | PRFISVGYVDDTQFVRFDSDAASPRGEPRAPWVEQEGPEYWD | |
| | | RETQKYKRQAQADRVSLRNLRGYYNQSEDGSHTLQRMSGCDL | |
| | | GPDGRLLRGYDQSAYDGKDYIALNEDLRSWTAADTAAQITQRK | |
| | | LEAARAAEQLRAYLEGTCVEWLRRYLENGKETLORAEPPKTHVT | |
| | | HHPLSDHEATLRCWALGFYPAEITLTWORDGEDQTQDTELVET | |
| | | RPAGDGTFQKWAAVVVPSGQEQRYTCHMQHEGLQEPLTLSW | |
| | | EPSSQPTIPIMGIVAGLAVLVVLAVLGAVVTAMMCRRKSSGGK | |
| | | GGSCSQAACSNSAQGSDESLITCKA | |
| HLA-C*0102 | HLA00401 | MRVMAPRTLILLLSGALALTETWACSHSMKYFFTSVSRPGRGEP | 96 |
| | | RFISVGYVDDTQFVRFDSDAASPRGEPRAPWVEQEGPEYWDR | |
| | | ETQKYKRQAQTDRVSLRNLRGYYNQSEAGSHTLQWMCGCDL | |
| | | GPDGRLLRGYDQYAYDGKDYIALNEDLRSWTAADTAAQITQRK | |
| | | WEAAREAEQRRAYLEGTCVEWLRRYLENGKETLQRAEHPKTHV | |
| | | THHPVSDHEATLRCWALGFYPAEITLTWQWDGEDQTQDTELV | |
| | | ETRPAGDGTFQKWAAVMVPSGEEQRYTCHVQHEGLPEPLTLR | |
| | | WEPSSQPTIPIVGIVAGLAVLAVLAVLGAVVAVVMCRRKSSGGK | |
| | | GGSCSQAASSNSAQGSDESLIACKA | |
| HLA-C*0701 | HLA00433 | MRVMAPRALLLLLSGGLALTETWACSHSMRYFDTAVSRPGRGE | 97 |
| | | PRFISVGYVDDTQFVRFDSDAASPRGEPRAPWVEQEGPEYWD | |
| | | RETQNYKRQAQADRVSLRNLRGYYNQSEDGSHTLQRMYGCDL | |
| | | GPDGRLLRGYDQSAYDGKDYIALNEDLRSWTAADTAAQITQRK | |
| | | LEAARAAEQLRAYLEGTCVEWLRRYLENGKETLQRAEPPKTHVT | |
| | | HHPLSDHEATLRCWALGFYPAEITLTWORDGEDQTQDTELVET | |
| | | RPAGDGTFQKWAAVVVPSGQEQRYTCHMQHEGLQEPLTLSW | |
| | | EPSSQPTIPIMGIVAGLAVLVVLAVLGAVVTAMMCRRKSSGGK | |
| | | GGSCSQAACSNSAQGSDESLITCKA | |
| HLA-C*0401 | HLA00420 | MRVMAPRTLILLLSGALALTETWAGSHSMRYFSTSVSWPGRGE | 98 |
| | | PRFIAVGYVDDTQFVRFDSDAASPRGEPREPWVEQEGPEYWD | |
| | | RETQKYKRQAQADRVNLRKLRGYYNQSEDGSHTLQRMFGCDL | |
| | | GPDGRLLRGYNQFAYDGKDYIALNEDLRSWTAADTAAQITQRK | |
| | | WEAAREAEQRRAYLEGTCVEWLRRYLENGKETLQRAEHPKTHV | |
| | | THHPVSDHEATLRCWALGFYPAEITLTWQWDGEDQTQDTELV | |
| | | ETRPAGDGTFQKWAAVVVPSGEEQRYTCHVQHEGLPEPLTLR | |
| | | WKPSSQPTIPIVGIVAGLAVLAVLAVLGAMVAVVMCRRKSSGG | |
| | | KGGSCSQAASSNSAQGSDESLIACKA | |
| HLA-C*0801 | HLA00445 | MRVMAPRTLILLLSGALALTETWACSHSMRYFYTAVSRPGRGE | 99 |
| | | PRFIAVGYVDDTQFVQFDSDAASPRGEPRAPWVEQEGPEYWD | |
| | | RETQKYKRQAQTDRVSLRNLRGYYNQSEAGSHTLQRMYGCDL | |
| | | GPDGRLLRGYNQFAYDGKDYIALNEDLRSWTAADTAAQITQRK | |
| | | WEAARTAEQLRAYLEGTCVEWLRRYLENGKKTLQRAEHPKTHV | |
| | | THHPVSDHEATLRCWALGFYPAEITLTWORDGEDQTQDTELVE | |
| | | TRPAGDGTFQKWAAVVVPSGEEQRYTCHVQHEGLPEPLTLRW | |
| | | GPSSQPTIPIVGIVAGLAVLAVLAVLGAVMAVVMCRRKSSGGK | |
| | | GGSCSQAASSNSAQGSDESLIACKA | |

In FIG. 2B, a targeting vector was designed, containing homologous arm sequences upstream and downstream of mouse B2M gene, and an “A fragment” encoding human B2M protein, a portion of human HLA-A protein, and a portion of mouse H2-D1 protein. The “A fragment” contains sequences from 5′ end to 3′ end that encode the following polypeptides: the signal peptide of human HLA-A; human B2M; a flexible linker polypeptide sequence; a portion of the human HLA-A protein; and a portion of the mouse H2-D1 protein. Due to the existence of the flexible linker polypeptide sequence, the protein can have the functional domains of human B2M and HLA-A protein. In FIG. 3B, the targeting vector is similar to the targeting vector used in FIG. 2B, except that the knock in fragment (similar to the “A fragment” in FIG. 2B) does not contain any sequences encoding moues H2-D1 protein, but contains a sequence encoding α1, α2, α3, transmembrane, and intracellular domains of human HLA-A protein.

### Preparation of Mice Expressing a Humanized MHC Complex Including HLA-A*0201

Based on the sequence information of HLA-A*0201 (SEQ ID NO: 6), genetically modified mice expressing a humanized MHC complex including HLA-A*0201 were prepared by the methods described above. As shown in the targeting strategy in FIG. 2B, a sequence (about 3.9 kb) of mouse B2M gene locus was replaced with a sequence (about 3.5 kb) encoding the signal peptide sequence of human HLA-A*0201 protein, human B2M, a flexible linker sequence, α1 and α2 domains of human HLA-A*0201 protein, and α3, transmembrane, and intracellular domains of mouse H2-D1 protein, thereby humanizing the mouse MHC complex. The targeting vector contains homologous arm sequences upstream and downstream of mouse B2M gene, and an “A fragment” containing HLA-A*0201 and B2M gene (or cDNA) fragments. Specifically, sequence of the upstream homologous arm is shown in SEQ ID NO: 1, and sequence of the downstream homologous arm is shown in SEQ ID NO: 2. The connection between the human HLA-A*0201 sequence and the mouse H2-D1 sequence was designed as: 5′-CGGGTACCAGGGGCCACGGGGCGCCTCCCTGATCGCCTGTAGATCTAGATCTGGATCCTGTGT GACATACCTGTACCTTGTCCTCCAGAGTCAGGGGCTGGGAGTCATTTTCTCTGGC-3′ (SEQ ID NO: 81), wherein the last “T” in sequence “GATCT” is the last nucleotide of the human HLA-A*0201 sequence, and the first “G” in sequence “GGATC” is the first nucleotide of the mouse H2-D1 sequence. The encoded fusion protein sequence is shown in SEQ ID NO: 61.

Alternatively, as shown in the schematic diagram of the targeting strategy in FIG. 3B, a sequence (about 3.9 kb) of mouse B2M gene locus was replaced with a sequence (about 4.4 kb) encoding the signal peptide sequence of human HLA-A*0201 protein, human B2M, a flexible linker sequence, and α1, α2, α3 domains, transmembrane, and intracellular domains of human HLA-A*0201 protein, thereby humanizing the mouse MHC complex. The targeting vector contains homologous arm sequences upstream and downstream of mouse B2M gene, and a “knock in fragment” containing HLA-A*0201 and B2M gene (or cDNA) fragments. Specifically, sequence of the upstream homologous arm is shown in SEQ ID NO: 1, and sequence of the downstream homologous arm is shown in SEQ ID NO: 2. The connection between the downstream of human HLA-A*0201 sequence and the mouse sequence was designed as: 5′-CATTCAACTGACATTTGTTGTCTGCTAGGGATATGACTGCTTTTGCGGCCTCGACAG AGCTCTGCGGGTACATCTTAGCCCTTTCAGTCTCTAGTTGGTAGGCACTAGATTTA-3′ (SEQ ID NO: 82), wherein the last “C” in sequence “GCCGC” is the last nucleotide of the human HLA-A*0201 sequence, and the first “G” in sequence “GACAG” is the first nucleotide of the mouse sequence. The encoded fusion protein sequence is shown in SEQ ID NO: 62.

### Preparation of Mice Expressing a Humanized MHC Complex Including HLA-A*2402

Based on the sequence information of HLA-A*2402 (SEQ ID NO: 10), genetically modified mice expressing a humanized MHC complex including HLA-A*2402 were prepared by the methods described above. As shown in the schematic diagram of the targeting strategy in FIG. 3B, a sequence (about 3.9 kb) of mouse B2M gene locus was replaced with a sequence (about 3.7 kb) encoding the signal peptide sequence of human HLA-A*2402 protein (part 1), human B2M, a flexible linker sequence, and α1, α2, α3 domains, transmembrane, and intracellular domains of human HLA-A*2402 protein (part 2), thereby humanizing the mouse MHC complex. The targeting vector contains homologous arm sequences upstream and downstream of mouse B2M gene, and a “knock in fragment” containing HLA-A*2402 and B2M gene (or cDNA) fragments. Specifically, sequence of the upstream homologous arm is shown in SEQ ID NO: 1, and sequence of the downstream homologous arm is shown in SEQ ID NO: 2. The connection between the human HLA-A*2402 part 1 sequence and the upstream of the human B2M-encoding sequence was designed as: 5′-CTCGGGGGCCCTGGCCCTGACCCAGACCTGGGCAATCCAGCGTACTCCAAAGATTC AGGTTTACTCACGTCATCCAGCAGAG-3′ (SEQ ID NO: 83), wherein the “A” in sequence “GGGCA” is the last nucleotide of the human HLA-A*2402 part I sequence, and the first “A” in sequence “ATCCA” is the first nucleotide of the human B2M-encoding sequence. The connection between the human B2M-encoding sequence, the flexible linker sequence, and the human HLA-A*2402 part 2 sequence was designed as: 5′-CATGTGACTTTGTCACAGCCCAAGATAGTTAAGTGGGATCGAGACATGGGAGGTGGC GGATCCGGCGGAGGCGGCTCGGGTGGCGGCGGCTCTGGCTCCCACTCCATGAGGTAT TTCTCCACATCCGTGTCCCGGCC-3′ (SEQ ID NO: 84), wherein the “G” in sequence “ACATG” is the last nucleotide of the human B2M-encoding sequence, and the first “G” in sequence “GGCTC” is the first nucleotide of the human HLA-A*2402 part 2 sequence. The connection between the downstream of the human HLA-A*2402 part 2 sequence and the mouse sequence was designed as: 5′-GCTGCTCCATTGTCCTTGGCCCTTCAGTAGAACCTTGTCCCACCAAGACCCGTGATT ACGACAGAGCTCTGCGGGTACATCTTAGCCCTTTCAGTCTCTAGTTGGTAGGCACTA GATTTACCTTCTGGAGGCTTCCGGACACTC-3′ (SEQ ID NO: 85), whereas the “C” in sequence “ATTAC” is the last nucleotide of the human HLA-A*2402 part 2 sequence, and the first “G” in sequence “GACAG” is the first nucleotide of the mouse sequence. The encoded fusion protein sequence is shown in SEQ ID NO: 65.

### Preparation of Mice Expressing a Humanized MHC Complex Including HLA-A*1101

Based on the sequence information of HLA-A*1101 (SEQ ID NO: 9), genetically modified mice expressing a humanized MHC complex including HLA-A*1101 were prepared by the methods described above. As shown in the schematic diagram of the targeting strategy in FIG. 3B, a sequence (about 3.9 kb) of mouse B2M gene locus was replaced with a sequence (about 3.7 kb) encoding the signal peptide sequence of human HLA-A*1101 protein (part 1), human B2M, a flexible linker sequence, and α1, α2, α3 domains, transmembrane, and intracellular domains of human HLA-A*1101 protein (part 2), thereby humanizing the mouse MHC complex. The targeting vector contains homologous arm sequences upstream and downstream of mouse B2M gene, and a “knock in fragment” containing HLA-A*1101 and B2M gene (or cDNA) fragments. Specifically, sequence of the upstream homologous arm is shown in SEQ ID NO: 1, and sequence of the downstream homologous arm is shown in SEQ ID NO: 2. The connection between the human HLA-A*1101 part 1 sequence and the upstream of the human B2M-encoding sequence was designed as: 5′-CCTGCTACTCTCGGGGGCCCTGGCCCTGACCCAGACCTGGGCGATCCAGCGTACTCC AAAGATTCAGGTTTACTCACGTCATC-3′ (SEQ ID NO: 86), wherein the last “G” in sequence “GGGCG” is the last nucleotide of the human HLA-A*1101 part 1 sequence, and the first “A” in sequence “ATCCA” is the first nucleotide of the human B2M-encoding sequence. The connection between the human B2M-encoding sequence, the flexible linker sequence, and the human HLA-A*1101 part 2 sequence was designed as: 5′-GACTTTGTCACAGCCCAAGATAGTTAAGTGGGATCGAGACATGGGAGGTGGCGGATC CGGCGGAGGCGGCTCGGGTGGCGGCGGCTCTGGCTCCCACTCCATGAGGTATTTCTA CACCTCCGTGTCCCGGC-3′ (SEQ ID NO: 87), wherein the “G” in sequence “ACATG” is the last nucleotide of the human B2M-encoding sequence, and the first “G” in sequence “GGCTC” is the first nucleotide of the human HLA-A*1101 part 2 sequence. The connection between the downstream of the human HLA-A*1101 part 2 sequence and the mouse sequence was designed as: 5′-CAGTTTCTGTTCCGGCCACCATGGGCTTTATGTGGTCACAGCTCACCTGGGTCATCTT TGCTGCTCCATGACAGAGTCTGCGGGTACATCTTAGCCCTTTCAGTCTCTAGTTGGT AGGCACTAGAT-3′ (SEQ ID NO: 88), whereas the last “T” in sequence “TCCAT” is the last nucleotide of the human HLA-A*1101 part 2 sequence, and the first “G” in sequence “GACAG” is the first nucleotide of the mouse sequence. The encoded fusion protein sequence is shown in SEQ ID NO: 64.

### Preparation of Mice Expressing a Humanized MHC Complex Including HLA-A*0302

Based on the sequence information of HLA-A*0302 (SEQ ID NO: 8), genetically modified mice expressing a humanized MHC complex including HLA-A*0302 were prepared by the methods described above. As shown in the schematic diagram of the targeting strategy in FIG. 3B, a sequence (about 3.9 kb) of mouse B2M gene locus was replaced with a sequence (about 4.3 kb) encoding the signal peptide sequence of human HLA-A*0302 protein (part 1), human B2M, a flexible linker sequence, and α1, α2, α3 domains, transmembrane, and intracellular domains of human HLA-A*0302 protein (part 2), thereby humanizing the mouse MHC complex. The targeting vector contains homologous arm sequences upstream and downstream of mouse B2M gene, and a “knock in fragment” containing HLA-A*0302 and B2M gene (or cDNA) fragments. Specifically, sequence of the upstream homologous arm is shown in SEQ ID NO: 1, and sequence of the downstream homologous arm is shown in SEQ ID NO: 2. The connection between the human HLA-A*0302 part 1 sequence and the upstream of the human B2M-encoding sequence was designed as: 5′-GGCCCTGGCCCTGACCCAGACCTGGGCGATCCAGCGTACTCCAAAGATTCAGGTTTA CTCACGTCATCCAGCA-3′ (SEQ ID NO: 89), wherein the last “G” in sequence “GGGCG” is the last nucleotide of the human HLA-A*0302 part 1 sequence, and the first “A” in sequence “ATCCA” is the first nucleotide of the human B2M-encoding sequence. The connection between the human B2M-encoding sequence, the flexible linker sequence, and the human HLA-A*0302 part 2 sequence was designed as: 5′-GTCACAGCCCAAGATAGTTAAGTGGGATCGAGACATGGGAGGTGGCGGATCCGGCG GAGGCGGCTCGGGTGGCGGCGGCTCTGGCTCCCACTCCATGAGGTATTTCTTCACAT CCGTGTCCCGGCC-3′ (SEQ ID NO: 90), wherein the “G” in sequence “ACATG” is the last nucleotide of the human B2M-encoding sequence, and the first “G” in sequence “GGCTC” is the first nucleotide of the human HLA-A*0302 part 2 sequence. The connection between the downstream of the human HLA-A*0302 part 2 sequence and the mouse sequence was designed as: 5′-GAATAGGGAGGTGCTCTACAGTGATCATTCATTCAACTGACATTTGTTGTCTGCTAG GGAGACAGAGCTCTGCGGGTACATCTTAGCCCTTTCAGTCTCTAGTTGGTAGGCACT AGATTTACC-3′ (SEQ ID NO: 91), whereas the last “A” in sequence “AGGGA” is the last nucleotide of the human HLA-A*0302 part 2 sequence, and the first “G” in sequence “GACAG” is the first nucleotide of the mouse sequence. The encoded fusion protein sequence is shown in SEQ ID NO: 63.

Humanized mice with other HLA genes in Table 17 can also be prepared by similar methods as described above, and the fusion proteins expressed by these humanized mice are shown in SEQ ID NOs: 66, 67, 68, 69, 70, 71, 72, 73, 74, 75, 76, 77, 78, 79, and 80 (via the targeting strategy in FIG. 3B), or SEQ ID NOs: 100, 101, 102, 103, 104, 105, 106, 107, 108, 109, 110, 111, 112, 113, 114, 115, 116, and 117 (via the targeting strategy in FIG. 2B).

### Detection of Human B2M and MHC Class I Expression in Mice

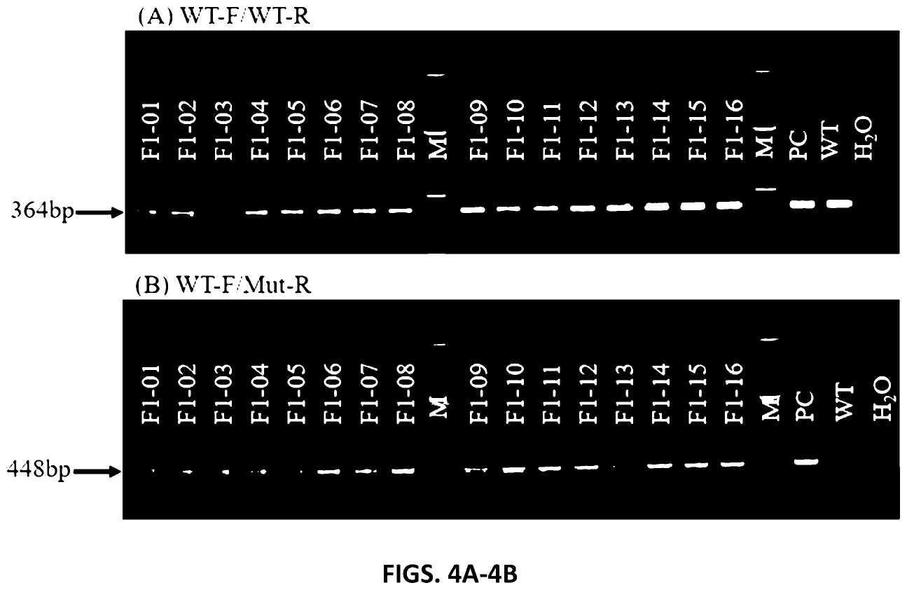

Genotypes of offspring mouse somatic cells (according to the targeting strategy of FIG. 3B) were determined by PCR and Southern Blot. For example, the PCR identification results of HLA-A*2402 heterozygous hVH/hVL mice (HLA-A*2402^{H/+} hVH/hVL) are shown in FIGS. 4A-4B. The results indicate that all 16 mice numbered F1-01 to F1-16 were positive heterozygous mice. The primers used in PCR identification are listed in the table below.

TABLE 18

| Primer | SEQ ID NO | Sequence (5′-3′) | Product size |
|---|---|---|---|
| WT-F | 16 | AGTTCAGCATCAACAGCTAGGAGAC | 364 bp |
| WT-R | 17 | TCCACCCTGTAGCCTCAAAG | |
| WT-F | 16 | AGTTCAGCATCAACAGCTAGGAGAC | 448 bp |
| Mut-R | 18 | AGTGGGGGTGAATTCAGTGTAG | |

Specifically, WT-F is located upstream of the 5′ homologous arm; WT-R is located within intron 1 of the mouse B2M gene; and Mut-R is located within the human B2M coding sequence.

The positive mice were further tested by Southern Blot to confirm whether there was random insertion. Genomic DNA was extracted from mouse tail blood, and restriction enzyme BclI or BglII was selected to digest the genomic DNA, followed by membrane transfer and hybridization. Both 5′ Probe and 3′ Probe are located within the HLA part 2 sequence. Specific probe and target fragment sizes are shown in the table below.

TABLE 19

| Restriction enzyme | Probe | WT size | Targeted size |
|---|---|---|---|
| BclI | 5′ Probe | 5.5 kb | 7.2 kb |
| BglII | 3′ Probe | 4.0 kb | 2.8 kb |

The following primers were used to synthesize Southern Blot probes:

5′ Probe-F:
(SEQ ID NO: 19)
5′-ATGAGGTCTTTTTGTGGGCAGAGCA-3′,
5′ Probe-R:
(SEQ ID NO: 20)
5′-CTCCCTACGGCCACATCACCATTAC-3′;
3′ Probe-F:
(SEQ ID NO: 21)
5′-TAACTTCATGTAAGGCACCGTCAC-3′,
3′ Probe-R:
(SEQ ID NO: 22)
5′-TCCAGACCTCACCATCAAATGAG-3′.

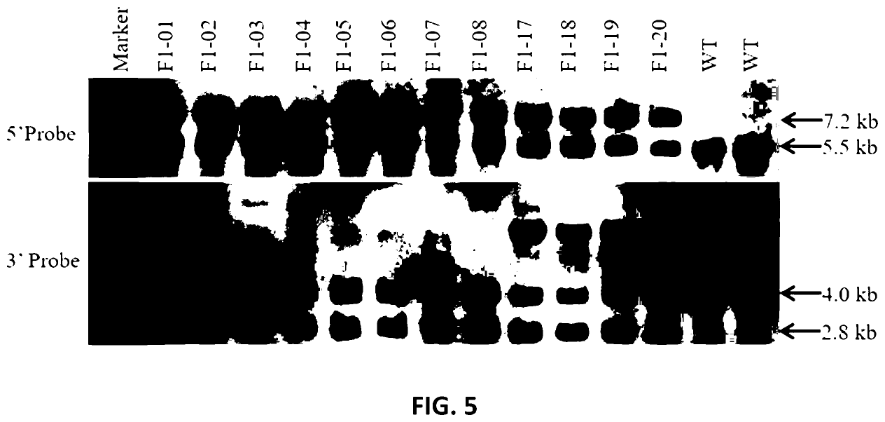

An exemplary Southern blot detection result is shown in FIG. 5. The result was obtained by using the 5′ Probe and 3′ Probe, which was further verified by sequencing. 8 mice numbered F1-01 to F1-08 were identified as positive clones without random insertions. The result indicates that MHC-I VH/VL mice can be generated using the method described herein, and the mice can be stably passed without random insertions. MHC-I VH/VL homozygous mice can be obtained by breeding the heterozygous mice identified as positive with each other.

Anti-mouse β2M antibody (BioLegend, Cat #: 154503), anti-human β2M antibody (BioLegend, Cat #: 395711), and anti-HLA-A3 antibody (Invitrogen Cat #: 17-5754-42) were used to detect the expression of human B2M protein and HLA protein in wild-type C57BL/6 mice and HLA-A*0302 humanized heterozygous mice (HLA-A*0302^{H/+} hVH/hVL) in vivo. The analysis results based on flow cytometry detection results are shown in the table below. The results indicate that the HLA-A*0302 hVH/hVL mice generated herein can successfully express human B2M protein and HLA protein in vivo.

TABLE 20

| Positive cells | C57BL/6 | HLA-A*0302^{H/+} hVH/hVL |
|---|---|---|
| Mouse B2M positive cells (%) | 43.4% | 10.5% |
| Human B2M positive cells (%) | 0.99% | 60.9% |
| HLA positive cells (%) | 0.17% | 75.2% |

Similarly, APC anti-human β2-microglobulin Antibody (BioLegend, Cat #: 395712), PE anti-mouse β2-microglobulin Antibody (BioLegend, Cat #: 154504), and PE anti-human HLA-A,B,C Antibody (BioLegend, Cat #: 311406) were used to detect the expression of human B2M protein and HLA protein in wild-type C57BL/6 mice and HLA-A*1101 humanized heterozygous mice (HLA-A*1101^{H/+} hVH/hVL) in vivo. The analysis results are shown in the table below. The results indicate that the HLA-A*1101 hVH/hVL mice generated herein can successfully express human B2M protein and HLA protein in vivo.

TABLE 21

| Positive cells | C57BL/6 | HLA-A*1101^{H/+} hVH/hVL |
|---|---|---|
| Mouse B2M positive cells (%) | 91.4% | 29.2% |
| Human B2M positive cells (%) | 0.65% | 96.8% |
| HLA positive cells (%) | 0.94% | 98.3% |

Similarly, the protein expression of humanized mice with other HLA genes in Table 17 can be detected by the methods described above. The results showed that the MHC-I VH/VL mice generated by the methods described herein can successfully express human B2M protein and HLA protein in vivo.

### Example 2: Generation of Human Antibodies

The MHC-I VH/VL mice obtained as described herein were immunized with antigen-MHC complexes. For example, the mice were immunized with antigen-MHC complexes multiple (e.g., four) times, and orbital blood was collected before immunization (as a negative control). Freund's complete adjuvant (CFA) was used for the first immunization, and Freund's incomplete adjuvant (IFA) was used for the second to fourth immunizations, with an interval of two weeks between each immunization. Orbital blood was collected 1 week after the third immunization, and serum titers of antigen-specific antibodies were detected by ELISA.

In subsequent experiments, MHC-I VH/VL mice were immunized with an antigen-MHC complex including one of the following heterologous antigens: Peptide A, Peptide B, Peptide C, Peptide D, Peptide E and Peptide F. These antigenic peptides are compatible with HLA-A*0201, and the immunized mice can express HLA-A*0201 protein.

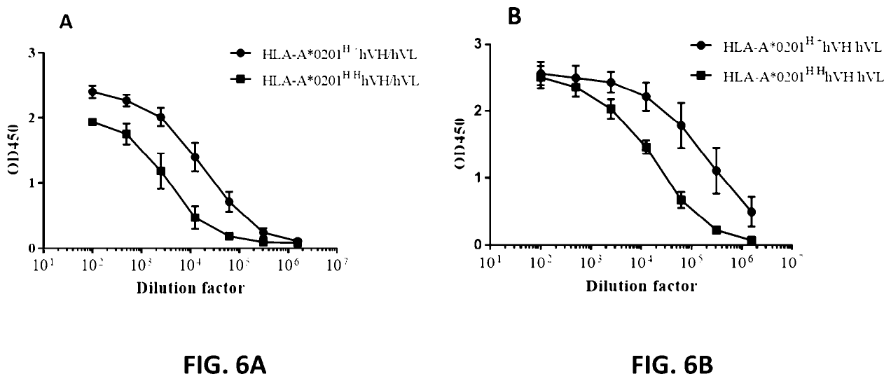

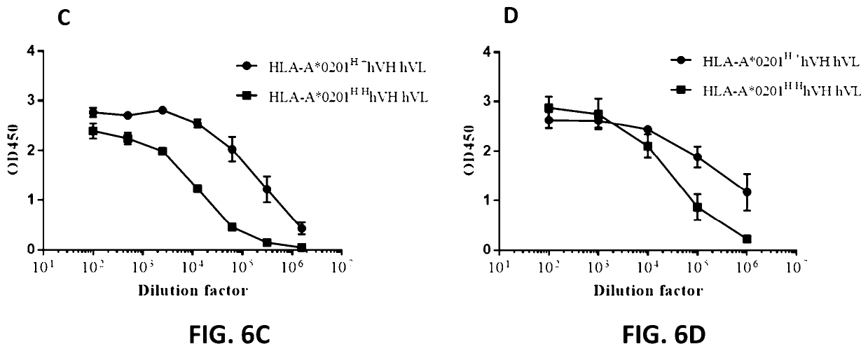

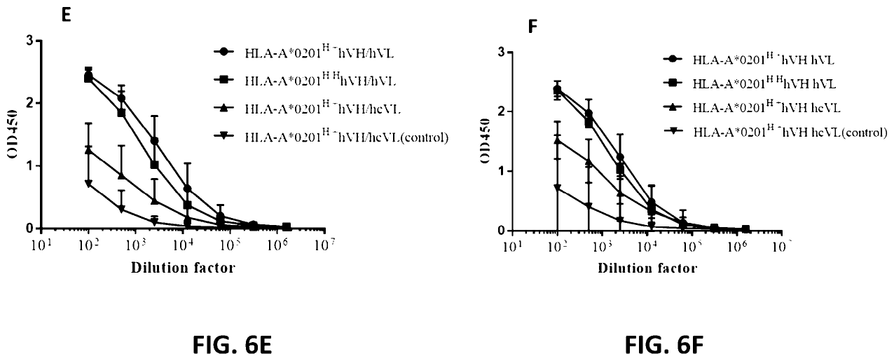

The ELISA was performed as follows. The His-tagged antigen was diluted in 1×PBS to 0.5 μg/ml and added to a 96-well plate with 0.1 ml/well, followed by an incubation at 37° C. for 2 hours. After the incubation, each well was washed with 300 μl 1×PBS three times, and then blocked by 250 μl 1×PBS supplemented with 5% non-fat milk at 37° C. for 1 hour. Each well was then washed by 300 μl 1×PBS twice. Serum samples from the MHC-I VH/VL mice were first diluted using 1×PBS at 1:100, 1:500, 1:2500, 1:12500, 1:62500, 1:312500, or 1:1562500, and then added to the 96-well plate. Serum samples from unimmunized mice were also diluted at 1:100 and added to the plate as a blank control. The diluted serum samples (120 μl/well) were incubated at 37° C. in the 96-well plate for 1 hour. After the incubation, the plate was washed with 300 μl/well of 1×PBST five times. The plate was then incubated with 0.1 ml/well of 1:20000 diluted goat-anti-mouse IgG Fc (HRP) at 37° C. for 1 hour. After the incubation, the plate was washed with 300 μl/well of 1×PBST five times. Next, 0.1 ml TMB (3,3′,5,5′-Tetramethylbenzidine) developing solution was added to each well and the plate was kept in dark at room temperature for 10 minutes, followed by adding 0.1 ml stop solution to each well. OD450 was measured by a plate reader and the standard OD values were calculated. Antigen-specific antibody serum titers after three immunizations of mice with antigen Peptide A, Peptide B, Peptide C, Peptide D, Peptide E and Peptide F are shown in FIGS. 6A-6F, respectively. The results showed that the antibody titer obtained by immunizing HLA heterozygous mice was higher than that of HLA homozygous mice.

Antigen-positive immune cells were isolated from immunized mice, and fused with mouse myeloma cells to form hybridoma cells. The hybridoma cells secreting antigen-specific monoclonal antibodies were screened, to obtain antigen-specific antibodies. The heavy chain and light chain variable region sequences of antigen-specific antibodies can also be directly obtained by isolating antigen-positive B cells from immunized mice. For example, single-cell technologies (e.g., Beacon® Optofluidic System, Berkeley Lights Inc.) can be used to screen and isolate plasma cells secreting antigen-specific monoclonal antibodies, followed by reverse transcription and PCR sequencing to obtain antibody variable region sequences. The sequences can then be used to express the antibodies. The specificity of the expressed antibody binding to the antigen peptide-MHC complex can then be verified by FACS (fluorescence activated cell sorting).

### Example 3: Diversity and Binding Affinity Distribution of Anti-Peptide A Antibodies

After immunization of mice with antigen Peptide A-MHC complex, Beacon® Optofluidic System was used to isolate the plasma cells that can produce antigen-specific monoclonal antibodies. Total RNA was extracted from the plasma cells, which was reverse transcribed into cDNA and then sequenced. A total of 171 pairs of antibody heavy chain variable region (VH) sequences and light chain variable region (VL) sequences were obtained. Sequences expressing the VH and VL of these antibodies were cloned into a vector expressing human IgG1 constant regions to obtain human antibodies.

Antibody binding to Peptide A-MHC complex was detected by FACS. Before FACS detection, antigen Peptide A-loaded T2 cells were prepared. Specifically, T2 cells (ATCC, Cat #: CRL-1992) were centrifuged at 25° C. for 5 minutes, and supernatant was discarded. Medium containing antigen Peptide A and human B2M protein (concentration was 20 μg/mL) was used to resuspend the cells to 2×10⁶ cells/ml. The cells were cultured at 37° C. for more than 16 hours under 5% CO₂ conditions. The control group was added with an irrelevant peptide (control peptide) and human B32M protein. Afterwards, the cells were collected by centrifugation and resuspended to an appropriate density (about 1×10⁵ cells in 10 μl) to obtain Peptide A-loaded T2 cells (T2 plus Peptide A) and irrelevant peptide-loaded T2 cells (T2 plus control peptide). 50 μl of the tested antibody was diluted to a specified concentration, and then 50 μl of a fluorescent antibody was added. The mixture was incubated at 4° C. for 30 minutes, and then washed once by PBS. The washed cells were resuspended and subjected to FACS analysis.

The above method was used, and 115 antigen-specific antibodies (characterized as: T2 negative; T2 plus Peptide A positive, and T2 plus control peptide negative) were screened from the 171 antibodies. Some exemplary flow cytometry detection results are shown in the table below.

TABLE 22

| | | T2 plus | T2 plus | Antigen-specific |
|---|---|---|---|---|
| Antibody | T2 | peptide A | control peptide | antibody |
| A-001 | 0.51% | 99.4% | 0.61% | Yes |
| A-002 | 0.30% | 73.6% | 0.41% | Yes |
| A-003 | 0 | 60.3% | 0 | Yes |
| A-004 | 7.52% | 10.0% | 2.42% | No |
| A-005 | 15.20% | 97.7% | 6.05% | Yes |
| A-006 | 0 | 89.2% | 0.19% | Yes |
| A-007 | 5.15% | 5.4% | 4.18% | No |
| A-008 | 0 | 70.0% | 0 | Yes |
| A-009 | 0.64% | 51.0% | 0.68% | Yes |
| A-010 | 0 | 69.7% | 0 | Yes |
| A-011 | 0.15% | 83.2% | 0.31% | Yes |
| A-012 | 16.4% | 3.8% | 4.94% | No |
| A-013 | 0.45% | 72.8% | 1.32% | Yes |
| A-014 | 0 | 90.6% | 0.2% | Yes |
| A-015 | 6.51% | 3.9% | 2.41% | No |
| A-016 | 0.16% | 79.3% | 0 | Yes |
| A-017 | 0.46% | 95.2% | 0.66% | Yes |
| A-018 | 0 | 78.2% | 0 | Yes |
| A-019 | 0.51% | 68.8% | 0.65% | Yes |
| A-020 | 0.10% | 96.5% | 0.55% | Yes |
| A-021 | 0.10% | 93.1% | 1.79% | Yes |
| A-022 | 0.24% | 99.5% | 2.58% | Yes |
| A-023 | 0.59% | 96.9% | 1.62% | Yes |
| A-024 | 7.90% | 3.3% | 1.56% | No |
| A-025 | 0.61% | 98.7% | 0.59% | Yes |
| A-026 | 0.07% | 91.8% | 0.50% | Yes |
| A-027 | 0.22% | 100% | 1.55% | Yes |
| A-028 | 0.12% | 95.4% | 0.55% | Yes |
| A-029 | 0.66% | 97.1% | 3.30% | Yes |
| A-030 | 0 | 89.3% | 0 | Yes |
| A-001 | 0.51% | 99.4% | 0.61% | Yes |

The Biacore™ system was then used to determine the affinity of these antibodies. Purified anti-antigen Peptide A antibodies were diluted to 1 μg/ml and then injected into the Biacore™ 8K biosensor at 10 μl/min for about 50 seconds to achieve a desired protein density (e.g., about 50 response units (RU)). His-tagged antigen Peptide A-MHC complex at a concentration of 200 nM was then injected at 30 μl/min for 120 seconds. Dissociation was monitored for 600 seconds. The chip was regenerated after the last injection of each titration with a glycine solution (pH 2.0, 30 μl/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

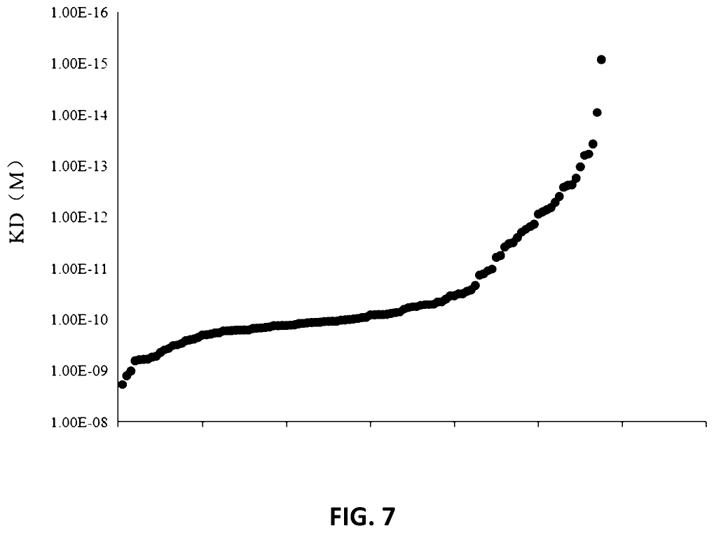

As a person of ordinary skill in the art would understand, the same method with appropriate adjustments for parameters (e.g., antibody concentrations) was performed for each tested antibody. As shown in FIG. 7, the result showed that 55.6% of the tested antibodies had a KD value at or below 10⁻⁸ M; 20% of the tested antibodies (23 out of 115) had a KD value of about 10⁻¹⁰; and 33.9% of the tested antibodies (39 out of 115) had a KD value of less than 10⁻¹¹. The result indicates that the methods described herein can successfully generate antibodies with high binding affinity against an antigen. Meanwhile, the VH and VL sequences of the 171 antibodies obtained from the first screening were aligned, and 16 pairs of antibody VH and VL sequences were obtained after deduplication.

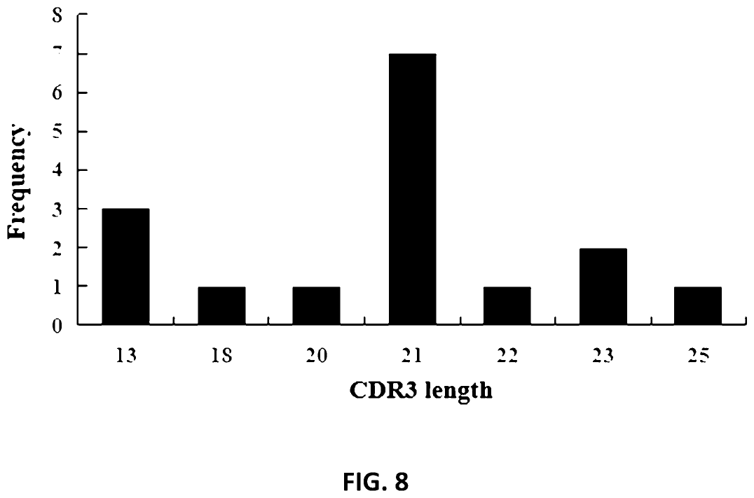

The VH and VL CDR3 sequence length and human germline genes usage of these antibodies were also determined. Length distribution of the VH CDR3 sequences is shown in FIG. 8. The result showed that 75% of the 16 antibodies had a VH CDR3 length greater than 20 amino acids. After further analysis of the VH CDR3 sequences, 10 groups of sister antibodies (i.e., sequences with a VH CDR3 identity no less than 85%) were found, in which group 1 included 6 sequences, group 2 included 2 sequences, and the other groups each included 1 sequence. The result indicates that a diverse collection of antibody sequences can be obtained using the method described herein.

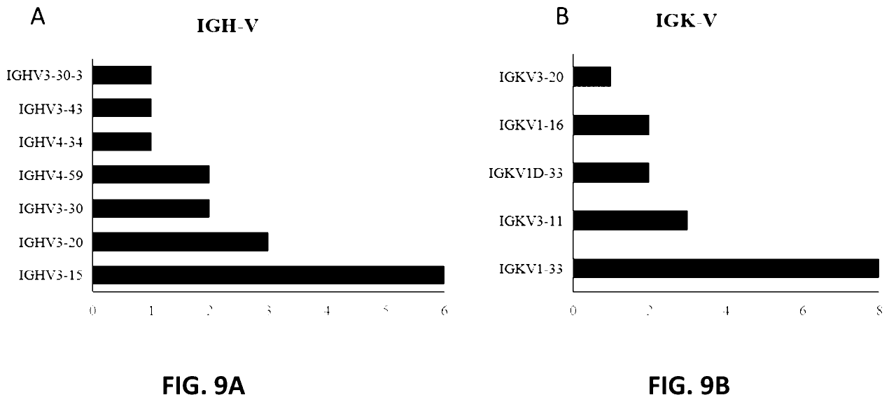

The human germline genes of the 16 antibody heavy chain sequences were confirmed by molecular biology methods, and then the variable region genes were classified into corresponding subgroups according to the IMGT database for statistical analysis. Human germline gene utilization results for these antibodies are shown in FIGS. 9A-9B. The results showed that the 16 antibodies involved 7 IGHV genes and 5 IGKV genes, indicating a diverse collection of antibodies.

### Example 4: Diversity and Binding Affinity Distribution of Anti-Peptide B Antibodies

In the antigen Peptide B group, a total of 269 pairs of VH and VL sequences were obtained. Similar methods as described in Example 3 was used, including high-throughput antigen peptide-loaded cell preparation and flow cytometry detection. The results showed that 34 antigen-specific antibodies were screened from the 269 antibodies. The binding affinity of the antibodies was also measured using the Biacore™ system. The positive cell percentages and affinities of some antibodies are shown in the table below.

TABLE 23

| | | | T2 plus | Antigen- | | | |
|---|---|---|---|---|---|---|---|
| | | T2 plus | control | specific | | Kon | koff |
| Antibody | T2 | peptide B | peptide | antibody | KD (M) | (1/Ms) | (1/s) |
| B-001 | 0.50% | 87.5% | 0.40% | Yes | 6.38E−09 | 5.09E+05 | 3.25E−03 |
| B-002 | 0.46% | 86.5% | 0.39% | Yes | 1.07E−08 | 2.29E+05 | 2.46E−03 |
| B-003 | 0.27% | 86.6% | 0.49% | Yes | 1.09E−08 | 4.16E+05 | 4.54E−03 |
| B-004 | 4.75% | 97.0% | 3.57% | Yes | 1.65E−08 | 2.55E+05 | 4.21E−03 |
| B-005 | 0.85% | 84.2% | 0.13% | Yes | 1.72E−08 | 4.18E+05 | 7.19E−03 |
| B-006 | 3.04% | 96.4% | 1.50% | Yes | 1.84E−08 | 2.43E+05 | 4.46E−03 |
| B-007 | 3.74% | 96.7% | 1.41% | Yes | 1.93E−08 | 2.05E+05 | 3.97E−03 |
| B-008 | 4.13% | 95.2% | 3.10% | Yes | 2.04E−08 | 2.67E+05 | 5.44E−03 |
| B-009 | 0.59% | 88.2% | 0.22% | Yes | 3.98E−08 | 2.12E+05 | 8.44E−03 |
| B-010 | 0.58% | 71.4% | 0.10% | Yes | 5.37E−08 | 9.32E+04 | 5.00E−03 |
| B-011 | 5.53% | 91.8% | 1.31% | Yes | 8.15E−08 | 5.72E+04 | 4.66E−03 |
| B-012 | 0.36% | 78.5% | 0.17% | Yes | 9.53E−08 | 4.09E+04 | 1.19E−02 |
| B-013 | 8.25% | 19.9% | 3.79% | Yes | 1.24E−07 | 8.49E+04 | 1.05E−02 |
| B-014 | 4.50% | 93.6% | 2.19% | Yes | 1.62E−07 | 4.09E+04 | 6.62E−03 |
| B-015 | 0.29% | 80.6% | 0.32% | Yes | 2.26E−07 | 1.20E+05 | 2.70E−02 |
| B-016 | 6.36% | 97.9% | 4.53% | Yes | 4.41E−07 | 1.85E+05 | 8.18E−02 |
| B-017 | 0.50% | 38.9% | 0.10% | No | — | — | — |
| B-018 | 4.10% | 85.8% | 1.48% | Yes | — | — | — |
| B-019 | 3.01% | 92.3% | 0.82% | Yes | 8.32E−07 | 1.21E+04 | 1.01E−02 |

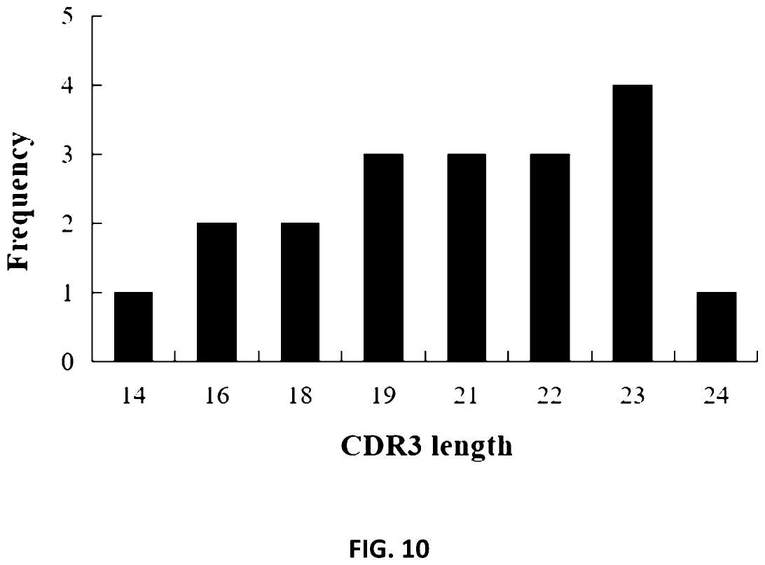

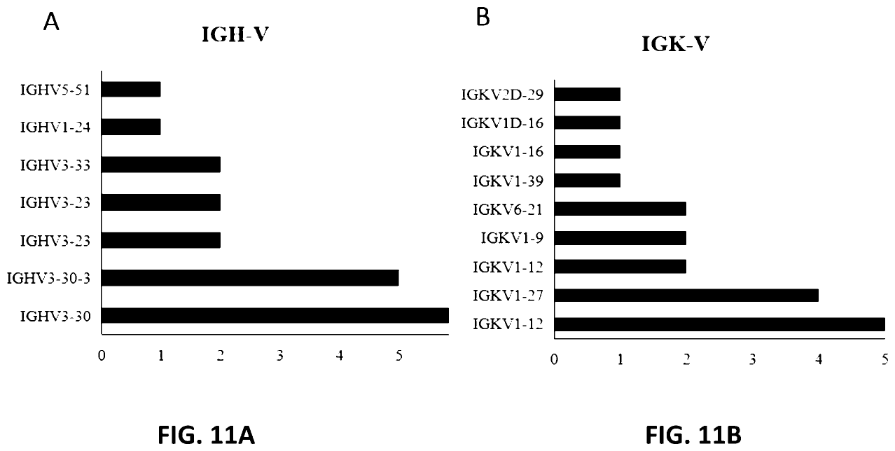

The VH, VL, and CDR sequences of all the antibodies were analyzed. After sequence alignment, 19 antibodies were obtained after deduplication. As shown in FIG. 10, 57.8% of the antibodies had a VH CDR3 length longer than 20 amino acids. The VH CDR3 sequences were further analyzed, and 17 groups of sister antibodies were found. Except for the group 2 and group 6, which both included 2 sequences, the other 15 groups each included only 1 sequence. Germline gene utilization is shown in FIGS. 11A-11B. The results showed that the 19 antibodies involved 7 IGHV genes and 9 IGKV genes, indicating a diverse collection of antibodies.

### Example 5: Diversity and Binding Affinity Distribution of Anti-Peptide C Antibodies

In the antigen Peptide C group, a total of 126 pairs of VH and VL sequences were obtained. Similar methods as described in Example 3 was used, including high-throughput antigen peptide-loaded cell preparation and flow cytometry detection. The results showed that 31 antigen-specific antibodies were screened from the 126 antibodies. The binding affinity of the antibodies was also measured using the Biacore™ system. Some exemplary flow cytometry detection and affinity results are shown in the table below.

TABLE 24

| | | | T2 plus | Antigen- | | | |
|---|---|---|---|---|---|---|---|
| | | T2 plus | control | specific | | Kon | koff |
| Antibody | T2 | peptide C | peptide | antibody | KD (M) | (1/Ms) | (1/s) |
| C-001 | 3.48% | 97.8% | 2.38% | Yes | 2.79E−09 | 1.93E+05 | 5.36E−04 |
| C-002 | 0.69% | 97.4% | 0.65% | Yes | 2.91E−09 | 1.43E+05 | 4.15E−04 |
| C-003 | 0 | 91.3% | 0.66% | Yes | 4.16E−09 | 1.38E+05 | 5.73E−04 |
| C-004 | 0.77% | 88.5% | 0.56% | Yes | 4.66E−09 | 1.39E+05 | 6.48E−04 |
| C-005 | 0.42% | 87.4% | 0.74% | Yes | 5.10E−09 | 1.38E+05 | 7.04E−04 |
| C-006 | 0.51% | 97.3% | 0.35% | Yes | 6.84E−09 | 2.05E+05 | 1.40E−03 |
| C-007 | 0.34% | 97.7% | 0.29% | Yes | 6.93E−09 | 2.81E+05 | 1.95E−03 |
| C-008 | 0.42% | 78.9% | 0.71% | Yes | 7.52E−09 | 1.29E+05 | 9.71E−04 |
| C-009 | 0.40% | 92.1% | 0.63% | Yes | 7.70E−09 | 6.02E+04 | 4.64E−04 |
| C-010 | 8.36% | 98.6% | 4.16% | Yes | 8.83E−09 | 8.10E+04 | 7.15E−04 |
| C-011 | 0.24% | 89.1% | 0.29% | Yes | 8.83E−09 | 5.34E+04 | 4.72E−04 |
| C-012 | 0.74% | 88% | 0.50% | Yes | 9.08E−09 | 8.40E+04 | 7.63E−04 |
| C-013 | 1.26% | 98% | 0.43% | Yes | 9.56E−09 | 6.93E+04 | 6.62E−04 |
| C-014 | 0.55% | 95.9% | 0.47% | Yes | 1.07E−08 | 5.25E+04 | 5.61E−04 |
| C-015 | 0.39% | 84.1% | 0.20% | Yes | 1.22E−08 | 5.81E+04 | 7.07E−04 |
| C-016 | 0.47% | 90.3% | 0.64% | Yes | 1.26E−08 | 5.21E+04 | 6.55E−04 |
| C-017 | 0.56% | 94% | 0.36% | Yes | 1.30E−08 | 1.54E+05 | 1.99E−03 |
| C-018 | 0.34% | 75.8% | 0.45% | Yes | 1.41E−08 | 5.50E+04 | 7.76E−04 |
| C-019 | 0.54% | 94.4% | 0.60% | Yes | 1.48E−08 | 1.63E+05 | 2.41E−03 |
| C-020 | 0.36% | 92.7% | 0.38% | Yes | 1.52E−08 | 1.53E+05 | 2.32E−03 |

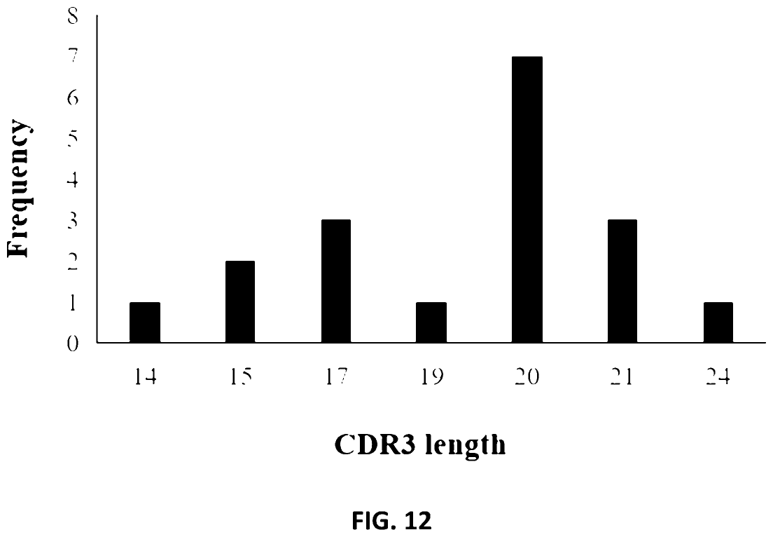

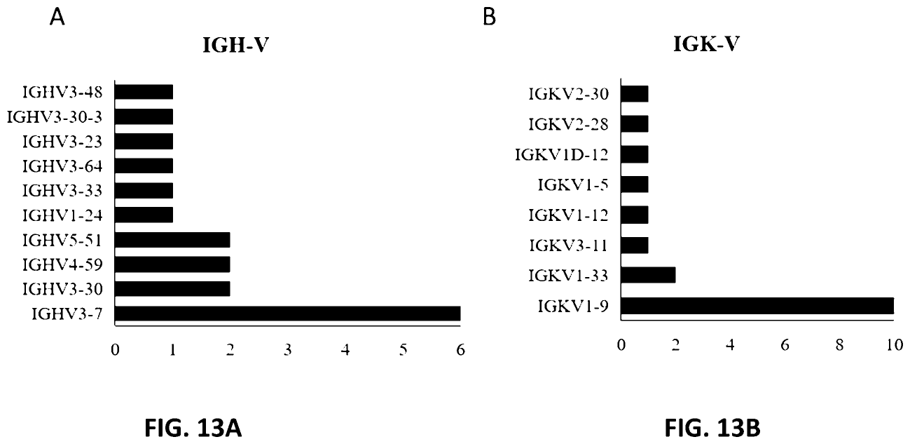

Similar to the method described in Example 3, the VH, VL, and CDR sequences of all the antibodies were analyzed. After sequence alignment, 18 antibodies were obtained after deduplication. As shown in FIG. 12, 11 antibodies had a VH CDR3 length longer than 20 amino acids. The VH CDR3 sequences were further analyzed, and 12 groups of sister antibodies were found. Except for the group 5 that included 6 sequences, the other 11 groups each included only 1 sequence. Germline gene utilization is shown in FIGS. 13A-13B. The results showed that the 18 antibodies involved 10 IGHV genes and 8 IGKV genes, indicating a diverse collection of antibodies.

The percentage of antibody-positive cells produced by HLA homozygous or heterozygous hVH/hVL mice (HLA^{H/+} hVH/hVL and HLA^{H/H} hVH/hVL) that were immunized with antigen Peptide B or antigen Peptide C was further analyzed, and the results are shown in the table below. The results showed that the percentage of antigen-specific antibody positive cells obtained by immunizing HLA heterozygous mice was higher than that of homozygous mice.

TABLE 25

| Antigen peptide | Mouse | No. of positive cells |
|---|---|---|
| Antigen Peptide B | HLA-A*0201^{H/+} hVH/hVL | 87 |
| | HLA-A*0201^{H/H} hVH/hVL | 44 |
| Antigen Peptide C | HLA-A*0201^{H/+} hVH/hVL | 177 |
| | HLA-A*0201^{H/H} hVH/hVL | 92 |

### Example 6: Diversity and Binding Affinity Distribution of Anti-Peptide D Antibodies

In the antigen Peptide D group, a total of 207 pairs of VH and VL sequences were obtained. 31 antigen-specific antibodies were screened using similar methods as described in Example 3 (e.g., flow cytometry detection). The binding affinity of the antibodies binding to antigen Peptide D was also measured using the Biacore™ system. Some exemplary results are shown in the table below. The results showed that the antibodies had high affinity to antigen peptide D, and KD of some antibodies was even below 10⁻⁸ M.

TABLE 26

| Antibody | KD (M) | Kon (1/Ms) | koff (1/s) |
|---|---|---|---|
| D-001 | 3.27E−08 | 7.64E+04 | 2.50E−03 |
| D-002 | 3.36E−08 | 7.85E+04 | 2.64E−03 |
| D-003 | 3.47E−08 | 8.68E+04 | 3.01E−03 |
| D-004 | 3.80E−08 | 6.98E+04 | 2.65E−03 |
| D-005 | 3.89E−08 | 7.81E+04 | 3.04E−03 |
| D-006 | 4.08E−08 | 6.42E+04 | 2.62E−03 |
| D-007 | 4.10E−08 | 8.36E+04 | 3.43E−03 |
| D-008 | 4.28E−08 | 1.34E+05 | 5.75E−03 |
| D-009 | 4.34E−08 | 6.28E+04 | 2.72E−03 |
| D-010 | 4.42E−08 | 6.99E+04 | 3.09E−03 |
| D-011 | 4.43E−08 | 7.42E+04 | 3.28E−03 |
| D-012 | 4.62E−08 | 7.15E+04 | 3.30E−03 |
| D-013 | 5.15E−08 | 2.12E+05 | 1.09E−02 |
| D-014 | 5.74E−08 | 7.37E+04 | 4.23E−03 |
| D-015 | 5.89E−08 | 8.64E+04 | 5.09E−03 |
| D-016 | 6.12E−08 | 9.25E+04 | 5.66E−03 |
| D-017 | 6.37E−08 | 6.22E+04 | 3.96E−03 |
| D-018 | 6.55E−08 | 5.89E+04 | 3.86E−03 |
| D-019 | 7.40E−08 | 1.35E+05 | 9.97E−03 |
| D-020 | 7.95E−08 | 3.07E+05 | 2.44E−02 |
| D-021 | 9.89E−08 | 7.93E+04 | 7.85E−03 |
| D-022 | 1.03E−07 | 7.82E+04 | 8.06E−03 |
| D-023 | 1.10E−07 | 1.02E+05 | 1.12E−02 |
| D-024 | 1.17E−07 | 1.01E+05 | 1.18E−02 |
| D-025 | 1.21E−07 | 1.13E+05 | 1.37E−02 |
| D-026 | 1.26E−07 | 1.17E+05 | 1.47E−02 |
| D-027 | 1.39E−07 | 8.27E+04 | 1.15E−02 |
| D-028 | 1.48E−07 | 8.07E+04 | 1.20E−02 |
| D-029 | 1.49E−07 | 9.26E+04 | 1.38E−02 |
| D-030 | 1.50E−07 | 1.12E+05 | 1.69E−02 |
| D-031 | 1.53E−07 | 1.04E+05 | 1.59E−02 |
| D-032 | 1.81E−07 | 7.23E+04 | 1.31E−02 |
| D-033 | 1.82E−07 | 6.23E+04 | 1.13E−02 |
| D-034 | 1.92E−07 | 9.43E+04 | 1.81E−02 |
| D-035 | 2.13E−07 | 1.24E+05 | 2.64E−02 |
| D-036 | 2.38E−07 | 7.38E+04 | 1.76E−02 |
| D-037 | 2.41E−07 | 1.27E+05 | 3.06E−02 |
| D-038 | 2.49E−07 | 1.23E+05 | 3.07E−02 |
| D-039 | 2.79E−07 | 6.97E+04 | 1.94E−02 |
| D-040 | 2.84E−07 | 1.17E+05 | 3.32E−02 |
| D-041 | 2.93E−07 | 1.09E+05 | 3.18E−02 |
| D-042 | 3.65E−07 | 1.03E+05 | 3.75E−02 |
| D-043 | 3.81E−07 | 9.56E+04 | 3.64E−02 |

The VH, VL, and CDR sequences of all the antibodies were analyzed. After sequence alignment, 15 antibodies were obtained after deduplication. Among them, 8 antibodies had a VH CDR3 of 20 amino acids, and 7 antibodies had a VH CDR3 of 19 amino acids. The VH CDR3 sequences were further analyzed, and 10 groups of sister antibodies were found: group 1 and group 2 had 3 sequences, groups 3-5 each included 2 sequences, and the other groups each had 1 sequence. The results indicate that a diverse collection of antibodies can be generated using the method described herein.

### Example 7: Diversity and Binding Affinity Distribution of Anti-Peptide F Antibodies

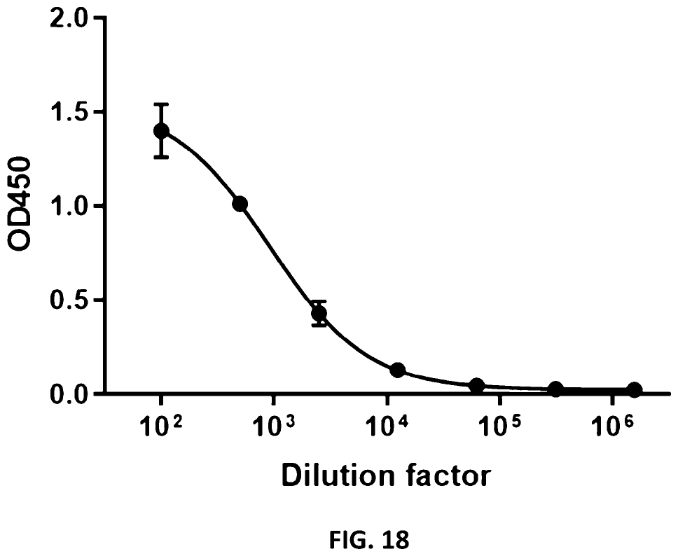

Similar to the methods described in Example 2, HLA-A*0201 hVH/hcVL heterozygous mice (HLA-A*0201^{H/+} hVH/hcVL v2) were immunized with a Peptide F-MHC complex. OD450 was measured by a plate reader and the standard OD values were calculated. Antigen-specific antibody serum titers after four immunizations of mice with antigen Peptide F are shown in FIG. 18.

After immunization of mice with antigen Peptide F-MHC complex, Beacon® Optofluidic System was used to isolate the plasma cells that can produce antigen-specific monoclonal antibodies. Total RNA was extracted from the plasma cells, which was reverse transcribed into cDNA and then sequenced. A total of 384 pairs of antibody heavy chain variable region (VH) sequences and light chain variable region (VL) sequences were obtained. Sequences expressing the VH and VL of these antibodies were cloned into a vector expressing human IgG1 constant regions to obtain human antibodies.

The methods described in Example 3 was used to detect antibody binding to Peptide F-MHC complex by FACS. 103 antigen-specific antibodies were screened from 384 antibodies. Some exemplary flow cytometry detection results are shown in the table below.

TABLE 27

| | | T2 plus | T2 plus control | Antigen-specific |
|---|---|---|---|---|
| Antibody | T2 | peptide F | peptide | antibody |
| F-001 | 1.4% | 90.8% | 3.3% | Yes |
| F-002 | 1.4% | 89.1% | 1.1% | Yes |
| F-003 | 1.1% | 92.6% | 1.2% | Yes |
| F-004 | 1.3% | 93.9% | 1.0% | Yes |
| F-005 | 1.4% | 86.7% | 96.8% | No |
| F-006 | 1.9% | 93.8% | 2.6% | Yes |
| F-007 | 1.4% | 87.6% | 1.2% | Yes |
| F-008 | 1.2% | 87.2% | 1.4% | Yes |

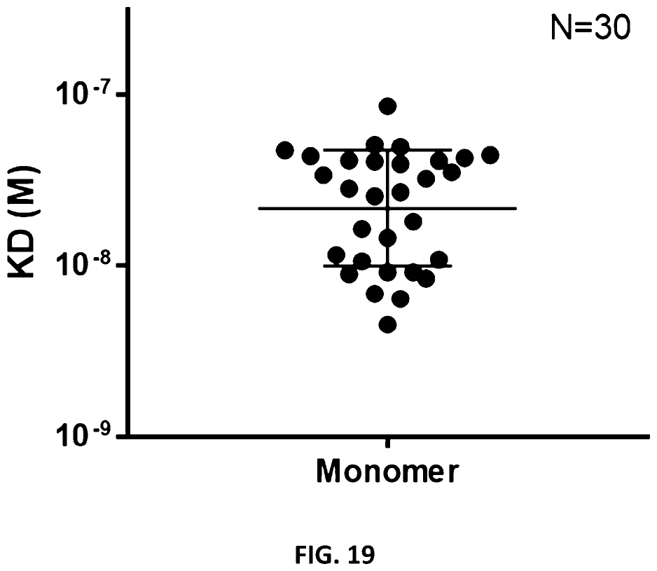

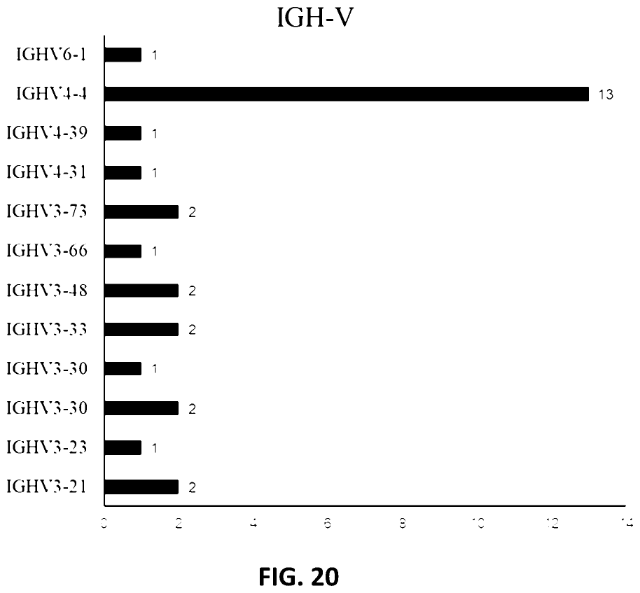

The methods described in Example 3 was used to measure antibody affinity using the Biacore™ system, and the results are shown in FIG. 19. In addition, human germline gene utilization results for these antibodies are shown in FIG. 20. The results showed that the 29 antibodies involved 12 IGHV genes, indicating a diverse collection of antibodies.

### Example 8: Diversity and Binding Affinity Distribution of Anti-Peptide G Antibodies

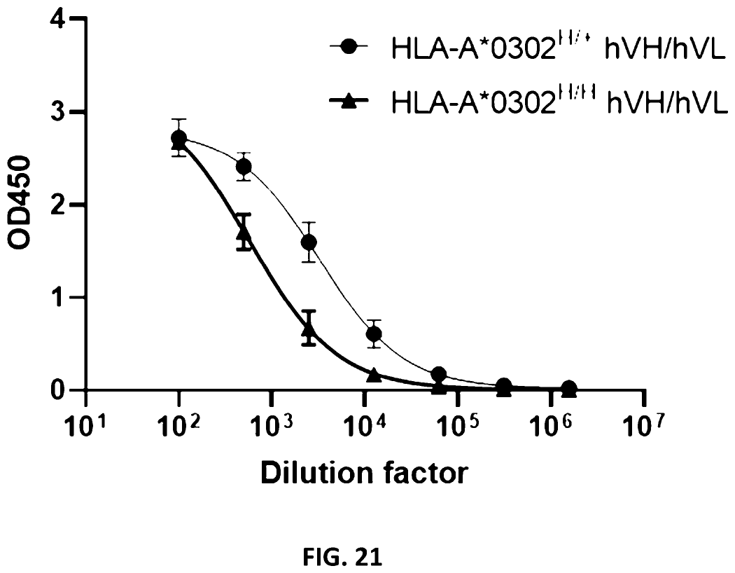

Similar to the methods described in Example 2, HLA-A*0302 hVH/hcVL homozygous mice (HLA-A*0302^{H/H} hVH/hcVL) and HLA-A*0302 hVH/hVL heterozygous mice (HLA-A*0302^{H/+} hVH/hVL) were immunized with a Peptide G-MHC complex. OD450 was measured by a plate reader and the standard OD values were calculated. Antigen-specific antibody serum titers after four immunizations of mice with antigen Peptide G are shown in FIG. 21. The results showed that the antibody titer obtained by immunizing HLA-A*0302 hVH/hVL heterozygous mice (HLA-A*0302^{H/+} hVH/hVL) was higher than that of HLA-A*0302 hVH/hVL homozygous mice (HLA-A*0302^{H/H} hVH/hVL).

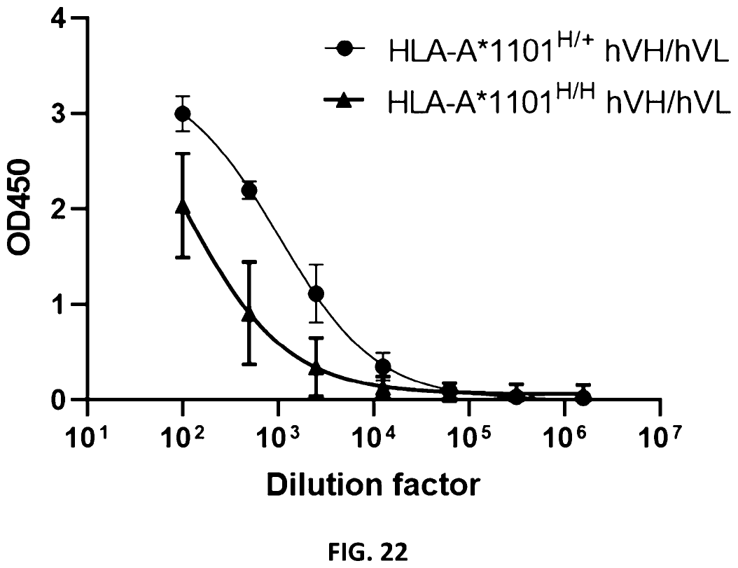

Using similar methods as described above, HLA-A*1101 hVH/hcVL homozygous mice (HLA-A*0101^{H/H} hVH/hcVL) and HLA-A*1101 hVH/hVL heterozygous mice (HLA-A*1101^{H/+} hVH/hVL) were immunized with a Peptide G-MHC complex. OD450 was measured by a plate reader and the standard OD values were calculated. Antigen-specific antibody serum titers after four immunizations of mice with antigen Peptide G are shown in FIG. 22. The results showed that the antibody titer obtained by immunizing HLA-A*1101 hVH/hVL heterozygous mice (HLA-A*1101^{H/+} hVH/hVL) was higher than that of HLA-A*1101 hVH/hVL homozygous mice (HLA-A*1101^{H/H} hVH/hVL).

A total of 289 pairs of antibody heavy chain variable region (VH) sequences and light chain variable region (VL) sequences were obtained. Sequences expressing the VH and VL of these antibodies were cloned into a vector expressing human IgG1 constant regions to obtain human antibodies.

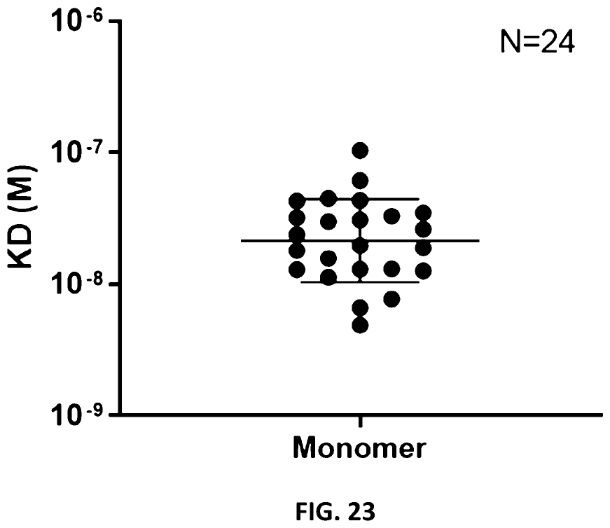

The methods described in Example 3 was used to detect antibody binding to Peptide G-MHC complex by FACS. 74 antigen-specific antibodies were screened from 289 antibodies. The antibody affinity was further measured using the Biacore™ system, and the results are shown in FIG. 23.

### Example 9: Diversity and Binding Affinity Distribution of Anti-Peptide H Antibodies

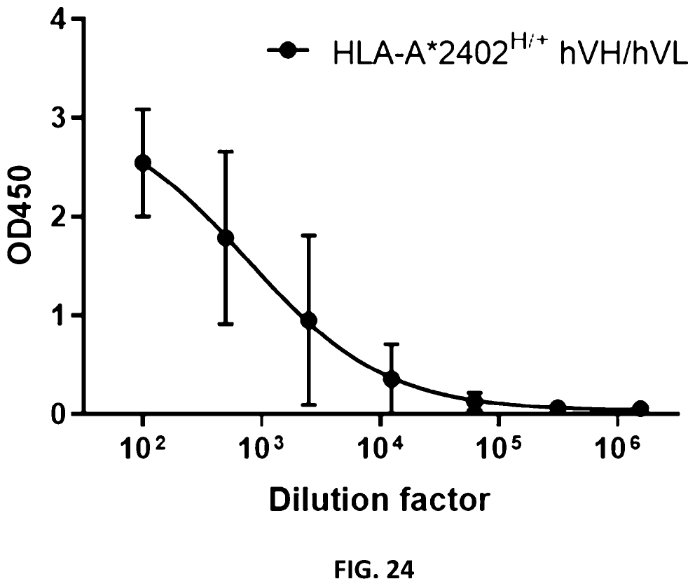

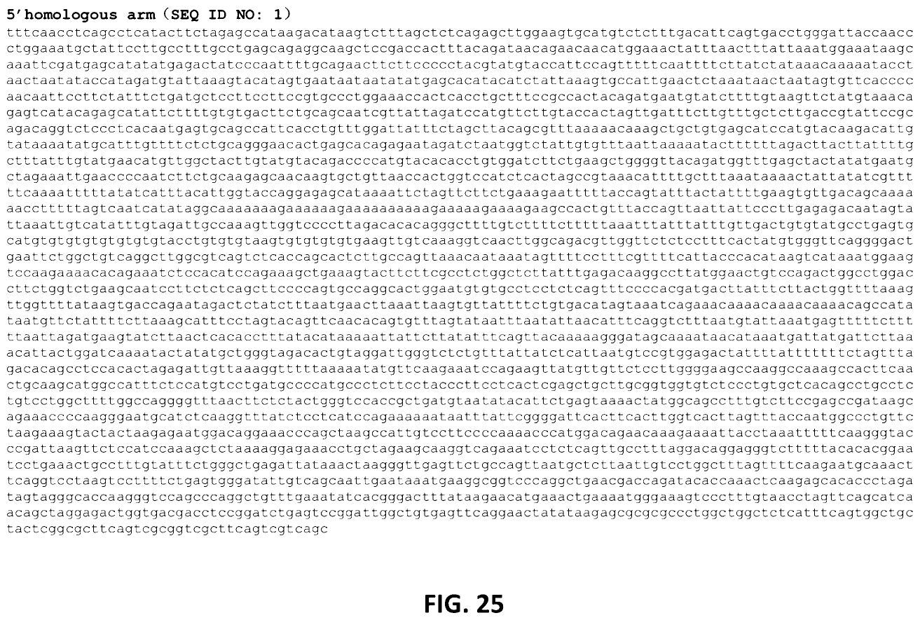

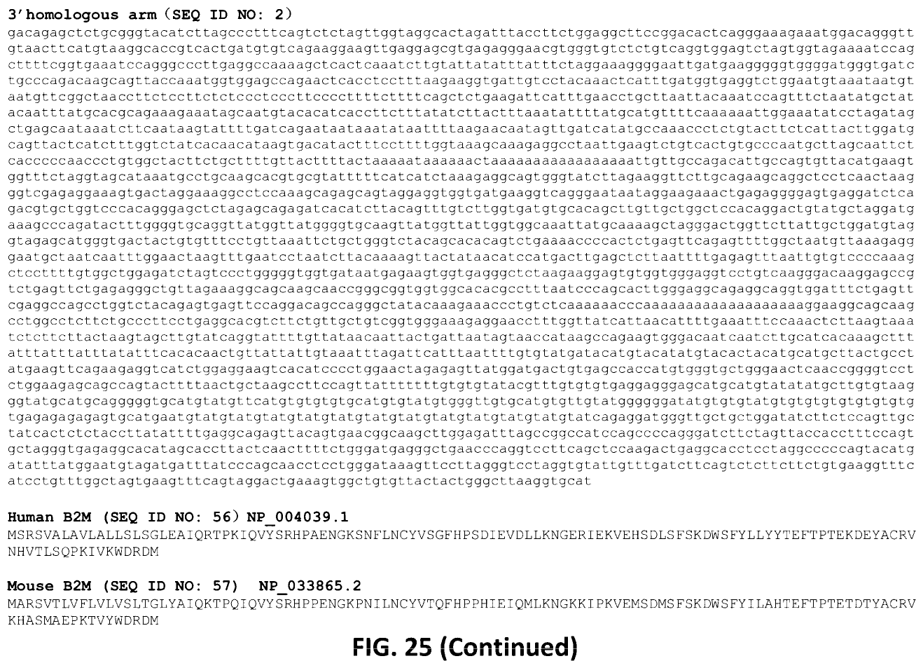

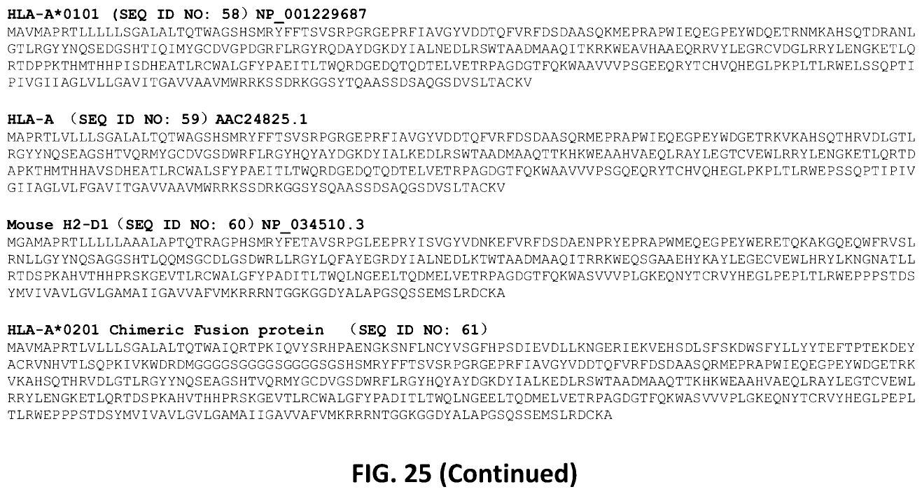

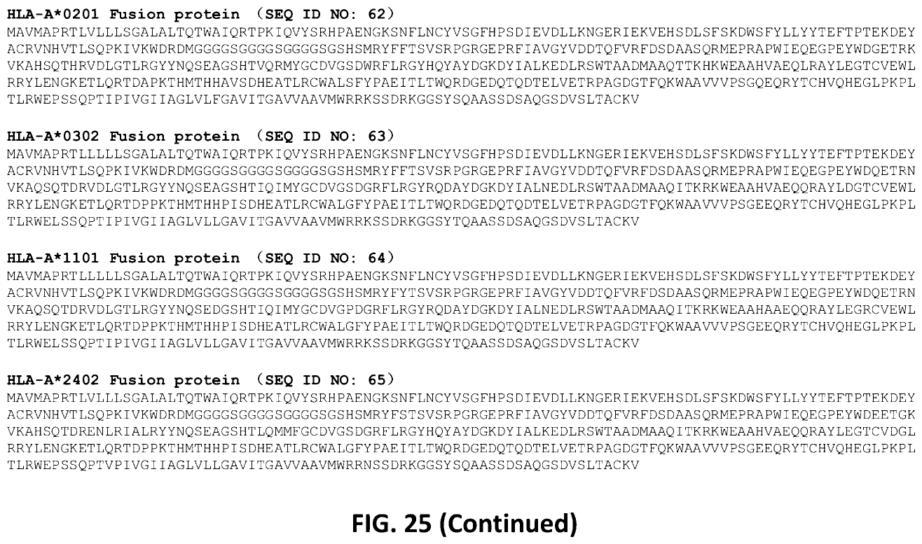

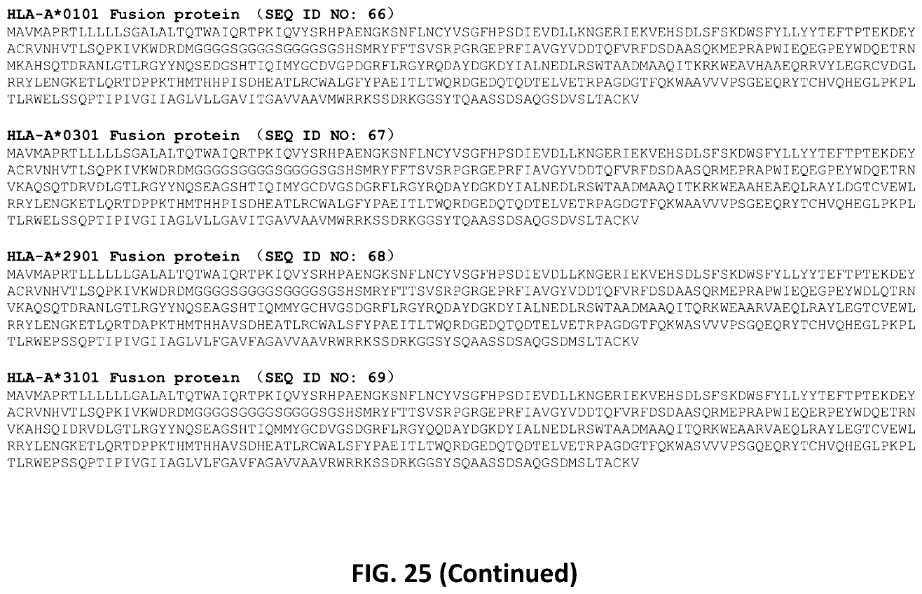

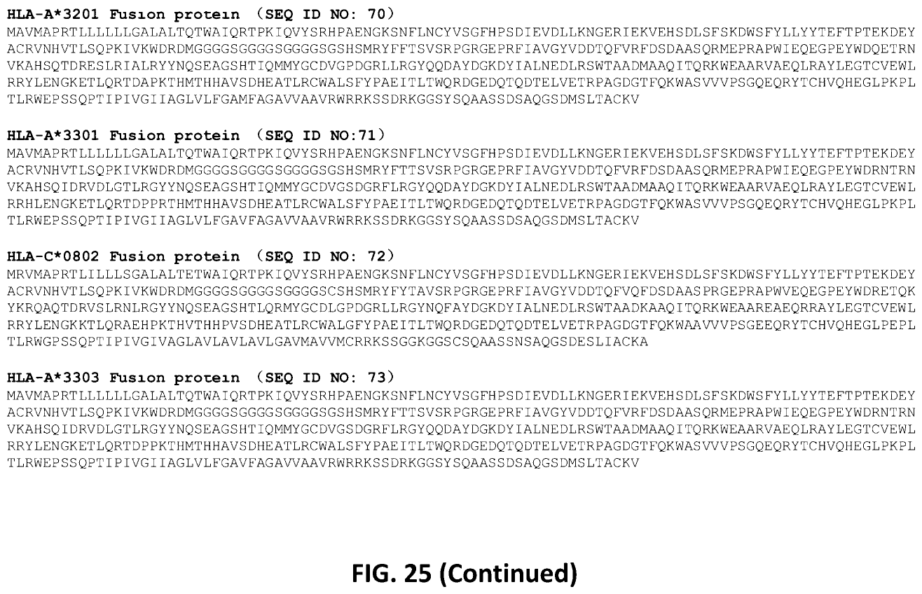

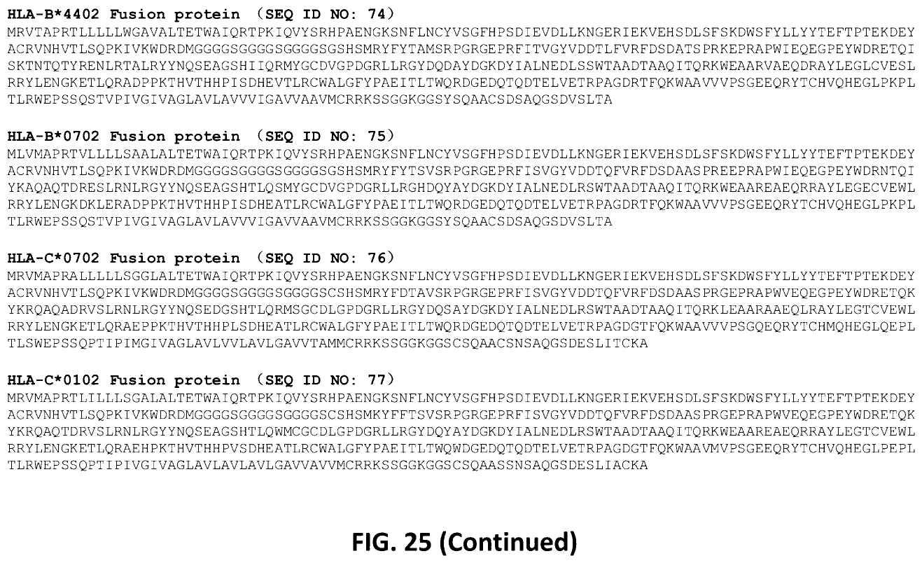

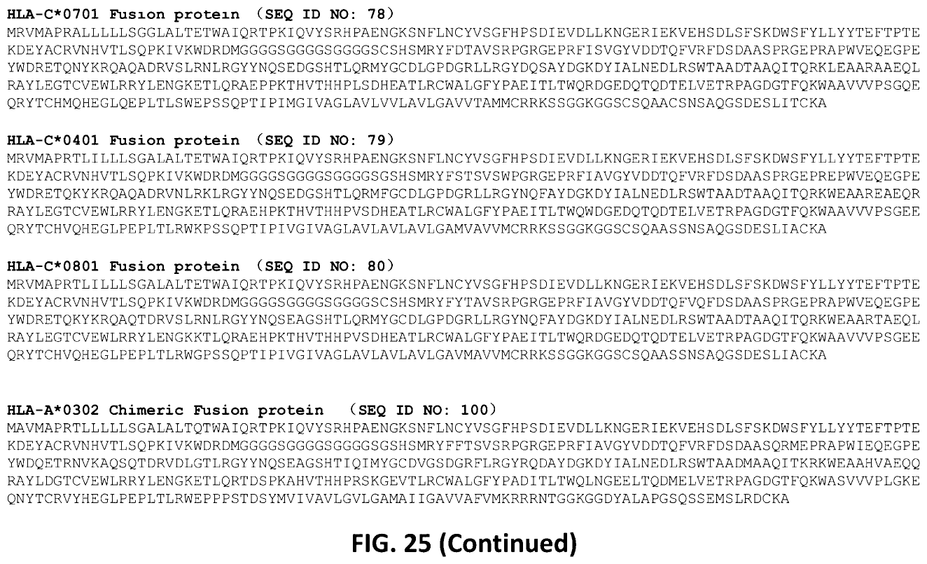

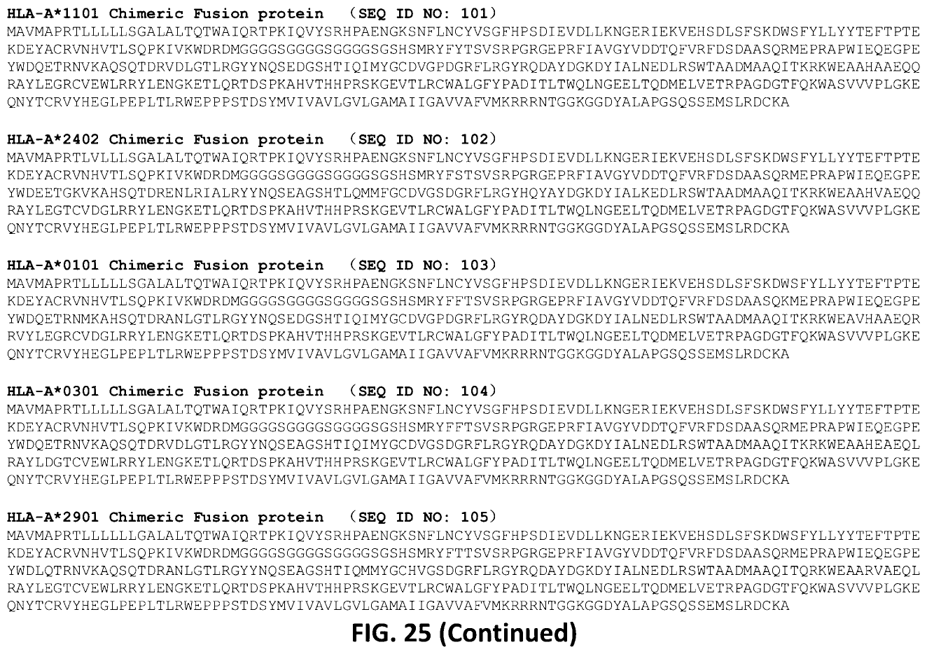

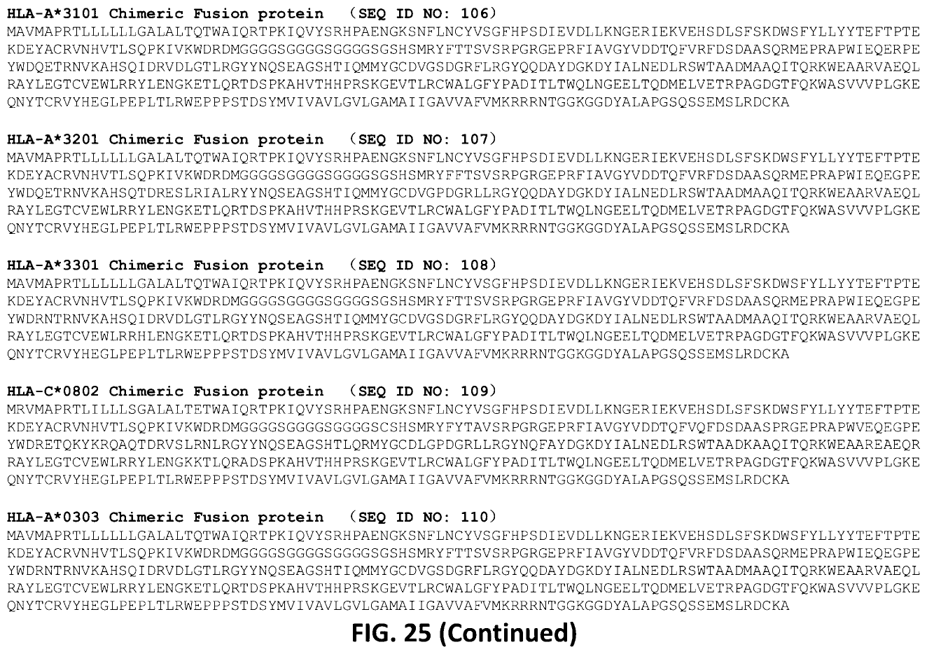

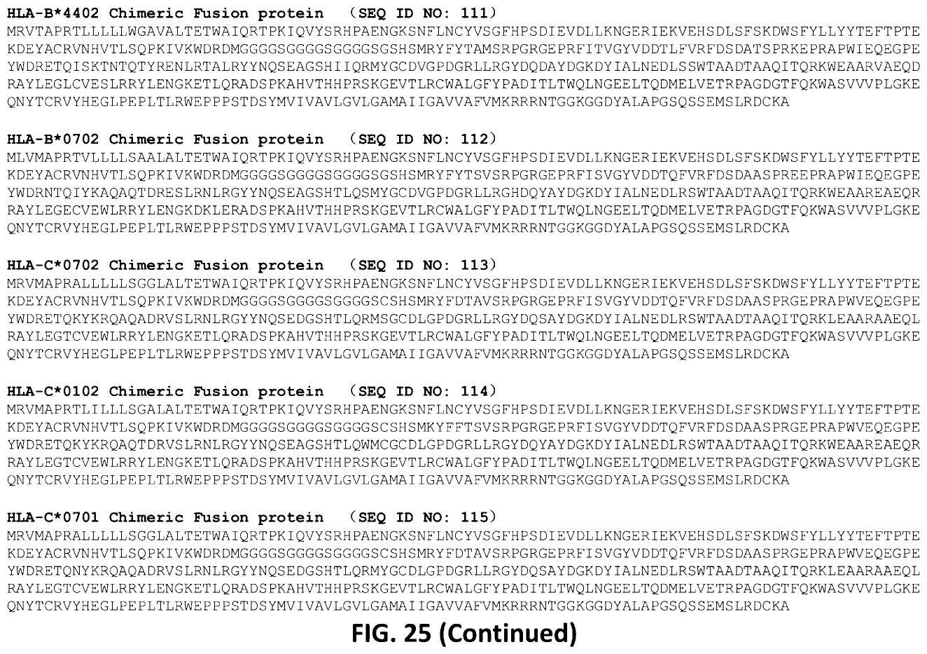

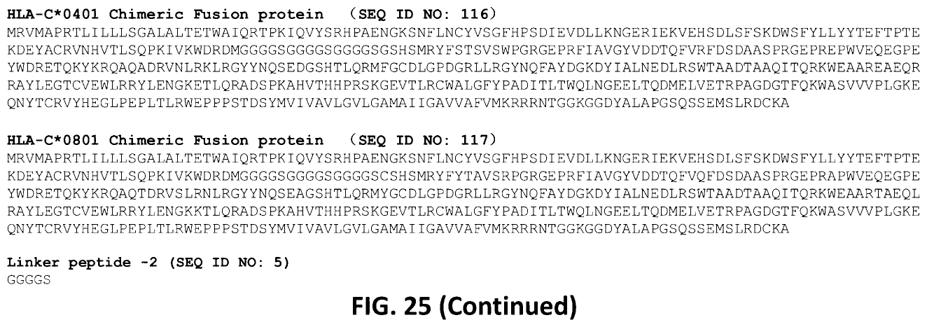

Similar to the methods described in Example 2, HLA-A*2402 hVH/hcVL heterozygous mice (HLA-A*2402^{H/+} hVH/hVL) were immunized with a Peptide H-MHC complex. OD450 was measured by a plate reader and the standard OD values were calculated. Antigen-specific antibody serum titers after four immunizations of mice with antigen Peptide G are shown in FIG. 24.

Using similar methods as described above, the Peptide H-MHC complex was used to immunize mice to produce antibodies. A total of 142 pairs of antibody heavy chain variable region (VH) sequences and light chain variable region (VL) sequences were obtained. Sequences expressing the VH and VL of these antibodies were cloned into a vector expressing human IgG1 constant regions to obtain human antibodies.

The methods described in Example 3 was used to detect antibody binding to Peptide H-MHC complex by FACS. Before that, Peptide H-loaded COS7 cells (COS7 plus peptide H) and control peptide-loaded COS7 cells (COS7 plus control peptide) were prepared. 24 antigen-specific antibodies were screened from 142 antibodies. Some exemplary flow cytometry detection results are shown in the table below.

TABLE 28

| | | COS7 plus | COS7 plus | Antigen-specific |
|---|---|---|---|---|
| Antibody | COS7 | Peptide H | control peptide | antibody |
| H-001 | 2.74% | 97.9% | 13.1% | Yes |
| H-002 | 5.26% | 92.7% | 5.25% | Yes |
| H-003 | 4.35% | 98.1% | 4.21% | Yes |
| H-004 | 7.11% | 98.0% | 4.42% | Yes |
| H-005 | 2.69% | 66.8% | 4.13% | Yes |
| H-006 | 8.37% | 98.9% | 13.8% | Yes |

The above results showed that the MHC humanized mice prepared herein can be used to generate antigen-specific fully human TCR-like antibodies. The generated TCR-like antibodies were highly diverse and had high affinity. Thus, the mice can be a useful mouse model for the development of TCR-like antibodies. At the same time, through high-throughput screening of the obtained antibodies, antibody molecules with high affinity and specificity can be quickly and efficiently obtained, which accelerates the discovery cycle of antibodies.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified non-human animal comprising at an endogenous heavy chain immunoglobulin gene locus, one or more human IGHV genes, one or more human IGHD genes, and one or more human IGHJ genes, wherein the human IGHV genes, the human IGHD genes, and the human IGHJ genes are operably linked and can undergo VDJ rearrangement, wherein the animal expresses a fusion protein comprising β2 microglobulin (B2M) and a human or humanized major histocompatibility complex (MHC) α chain.

2. The animal of claim 1, wherein the animal comprises at least 150 human IGHV genes selected from Table 1, at least 20 human IGHD genes selected from Table 2, and at least 5 human IGHJ genes selected from Table 3.

3. The animal of claim 1, wherein the animal comprises all human IGHV genes, all human IGHD genes, and all human IGHJ genes at the endogenous heavy chain immunoglobulin gene locus of human chromosome 14 of a human subject.

4. The animal of claim 1, wherein the animal comprises all human IGHV genes, all human IGHD genes, and all human IGHJ genes at the endogenous heavy chain immunoglobulin gene locus of human chromosome 14 of a human cell.

5. The animal of claim 1, wherein the animal comprises a disruption in the animal's endogenous heavy chain immunoglobulin gene locus.

6. The animal of claim 5, wherein the animal is a mouse and the disruption in the animal's endogenous heavy chain immunoglobulin gene locus comprises a deletion of one or more mouse IGHV genes in Table 4, one or more mouse IGHD genes in Table 5, and/or one or more mouse IGHJ genes in Table 6.

7. The animal of claim 5, wherein the animal is a mouse and the disruption in the animal's endogenous heavy chain immunoglobulin gene locus comprises a deletion of a contiguous sequence starting from mouse IGHV1-85 gene to mouse IGHJ4 gene.

8. The animal of claim 1, wherein the animal comprises one or more endogenous IGHM, IGHδ, IGHG3, IGHG1, IGHG2b, IGHG2a, IGHE, and IGHA genes.

9. The animal of claim 1, wherein the animal comprises an unmodified human sequence derived from a human heavy chain immunoglobulin gene locus, wherein the unmodified human sequence is at least 800 kb.

10. The animal of claim 1, wherein the animal comprises an unmodified human sequence derived from a human heavy chain immunoglobulin gene locus starting from human IGHV(III)-82 to human IGHV1-2.

11. The animal of claim 1, wherein the animal comprises an unmodified human sequence derived from a human heavy chain immunoglobulin gene locus starting from human IGHV(III)-82 to human IGHV6-1.

12. The animal of claim 1, wherein the animal comprises an unmodified human sequence derived from a human heavy chain immunoglobulin gene locus starting from human IGHD1-1 to human IGHJ6.

13. The animal of claim 1, wherein the animal comprises an unmodified human sequence derived from a human heavy chain immunoglobulin gene locus starting from human IGHV(III)-82 to human IGHJ6.

14. The animal of claim 1, wherein the animal is homozygous with respect to the heavy chain immunoglobulin gene locus.

15. The animal of claim 1, wherein the animal is heterozygous with respect to the heavy chain immunoglobulin gene locus.

16. The animal of claim 1, wherein the animal further comprises at an endogenous light chain immunoglobulin gene locus, one or more human IGKV genes, and one or more human IGKJ genes.

17. The animal of claim 1, wherein the animal comprises a disruption in the animal's endogenous lambda light chain immunoglobulin gene locus.

18. The animal of claim 1, wherein the animal is a rodent (e.g., a mouse).

19. A genetically-modified non-human animal comprising at an endogenous light chain immunoglobulin gene locus, one or more human IGKV genes and one or more human IGKJ genes, wherein the animal expresses a fusion protein comprising β2 microglobulin (B2M) and a human or humanized major histocompatibility complex (MHC) α chain.

20. The animal of claim 19, wherein the animal comprises all human IGKV genes in Table 7, and all human IGKJ genes in Table 8.

21. The animal of claim 19, wherein the animal comprises an unmodified sequence derived from a human light chain immunoglobulin gene locus starting from human IGKV3D-7 to human IGKJ5.

22. The animal of claim 19, wherein the animal comprises a disruption in the animal's endogenous light chain immunoglobulin gene locus.

23. The animal of claim 19, wherein the animal is a mouse and the disruption in the animal's endogenous light chain immunoglobulin gene locus comprises a deletion of one or more mouse IGKV genes in Table 9 and one or more mouse IGKJ genes in Table 10.

24. The animal of claim 22, wherein the animal is a mouse and the disruption in the animal's endogenous light chain immunoglobulin gene locus comprises a deletion of a sequence starting from mouse IGKV2-137 to mouse IGKJ5.

25. The animal of claim 19, wherein the animal comprises an endogenous IGKC.

26. The animal of claim 19, wherein the animal is homozygous with respect to the light chain immunoglobulin gene locus.

27. The animal of claim 19, wherein the animal is heterozygous with respect to the light chain immunoglobulin gene locus.

28. The animal of claim 19, wherein the animal further comprises at an endogenous heavy chain immunoglobulin gene locus, one or more human IGHV genes, one or more human IGHD genes, and one or more human IGHJ genes.

29. The animal of claim 19, wherein the animal comprises a disruption in the animal's endogenous lambda light chain immunoglobulin gene locus.

30. The animal of claim 19, wherein the animal is a rodent (e.g., a mouse).

31. A genetically-modified non-human animal comprising at the endogenous light chain immunoglobulin locus, an exogenous light chain variable region gene sequence, wherein the exogenous light chain variable region gene sequence comprises no more than three human IGKV genes and no more than two human IGKJ genes, wherein the no more than three human IGKV genes and the no more than two human IGKJ genes are operably linked to an endogenous light chain constant domain gene, wherein the animal expresses a fusion protein comprising β2 microglobulin (B2M) and a human or humanized major histocompatibility complex (MHC) α chain.

32. The animal of claim 31, wherein the no more than three human IGKV genes are selected from Table 7, and the no more than two human IGKJ genes are selected from Table 8.

33. The animal of claim 31 or 32, wherein the exogenous light chain variable region gene sequence comprises one human IGKV gene and one human IGKJ gene.

34. The animal of any one of claims 31-33, wherein the exogenous light chain variable region gene sequence further comprises a human IGKJ 3′-UTR sequence.

35. The animal of any one of claims 31-34, wherein the exogenous light chain variable region gene in one or more cells of the animal can subject to somatic hypermutations.

36. The animal of claim 35, wherein the somatic hypermutations can result in up to one, two, or three amino acid changes in light chain variable regions in the one or more cells of the animal.

37. The animal of any one of claims 31-36, wherein exogenous light chain variable region gene sequence comprises one human IGKV gene and one human IGKJ gene, wherein the human IGKV gene is selected from the group consisting of IGKV3-20, IGKV3-11, and IGKV1-39, wherein the human IGKV gene and the human IGKJ gene are operably linked.

38. The animal of claim 37, wherein the human IGKV gene is IGVK3-11.

39. The animal of any one of claims 31-38, wherein the human IGKJ gene is selected from the group consisting of IGKJ1 and IGKJ4.

40. The animal of any one of claims 31-39, wherein the human IGKV gene is IGKV1-39 and the human IGKJ gene is IGKJ4.

41. The animal of any one of claims 31-39, wherein the human IGKV gene is IGKV3-11 and the human IGKJ gene is IGKJ1.

42. The animal of any one of claims 31-39, wherein the human IGKV gene is IGKV3-20 and the human IGKJ gene is IGKJ1.

43. The animal any one of claims 31-42, wherein the animal further comprises a promoter sequence that is operably linked to the human IGKV gene, wherein the promoter sequence is within 2500 or 3000 bp of the human IGKV gene.

44. The animal of claim 43, wherein the promoter is an IGKV3-20 promoter, an IGKV3-11 promoter, or an IGKV1-39 promoter.

45. The animal of any one of claims 31-44, wherein the animal comprises a disruption in the animal's endogenous light chain immunoglobulin gene locus.

46. The animal of claim 45, wherein the animal is a mouse and the disruption in the animal's endogenous light chain immunoglobulin gene locus comprises a deletion of one or more mouse IGKV genes in Table 9 and one or more mouse IGKJ genes in Table 10.

47. The animal of claim 46, wherein the animal is a mouse and the disruption in the animal's endogenous light chain immunoglobulin gene locus comprises a deletion of a sequence starting from mouse IGKV2-137 to mouse IGKJ5.

48. The animal of any one of claims 31-47, wherein the animal comprises an endogenous IGKC.

49. The animal of any one of claims 31-48, wherein the animal further comprises a kappa intronic enhancer 5′ with respect to the endogenous IGKC and/or a kappa 3′ enhancer.

50. The animal of any one of claims 31-49, wherein the human light chain variable region is a rearranged sequence.

51. The animal of any one of claims 31-50, wherein the animal is homozygous with respect to the light chain immunoglobulin gene locus.

52. The animal of any one of claims 31-50, wherein the animal is heterozygous with respect to the light chain immunoglobulin gene locus.

53. The animal of any one of claims 31-52, wherein the animal comprises a disruption in the animal's endogenous lambda light chain immunoglobulin gene locus.

54. The animal of any one of claims 31-53, wherein the animal is a rodent (e.g., a mouse).

55. The animal of any one of claims 31-54, wherein the animal further comprises at an endogenous heavy chain immunoglobulin gene locus, one or more human IGHV genes, one or more human IGHD genes, and one or more human IGHKJ genes, wherein the human IGHV genes, the human IGHD genes, and the human IGHJ genes are operably linked and can undergo VDJ rearrangement.

56. The animal of claim 55, wherein the animal comprises at least 150 human IGHV genes selected from Table 1, at least 20 human IGHD genes selected from Table 2, and at least 5 human IGHJ genes selected from Table 3.

57. The animal of claim 55, wherein the animal comprises all human IGHV genes, all human IGHD genes, and all human IGHJ genes at the endogenous heavy chain immunoglobulin gene locus of human chromosome 14 of a human subject.

58. The animal of claim 55, wherein the animal comprises all human IGHV genes, all human IGHD genes, and all human IGHJ genes at the endogenous heavy chain immunoglobulin gene locus of human chromosome 14 of a human cell.

59. The animal of any one of claims 55-58, wherein the animal comprises an unmodified human sequence derived from a human heavy chain immunoglobulin gene locus, wherein the unmodified human sequence is at least 800 kb.

60. The animal of any one of claims 55-59, wherein the animal comprises an unmodified human sequence derived from a human heavy chain immunoglobulin gene locus starting from human IGHV(III)-82 to human IGHV1-2.

61. The animal of any one of claims 55-59, wherein the animal comprises an unmodified human sequence derived from a human heavy chain immunoglobulin gene locus starting from human IGHV(III)-82 to human IGHV6-1.

62. The animal of any one of claims 55-61, wherein the animal comprises an unmodified human sequence derived from a human heavy chain immunoglobulin gene locus starting from human IGHD1-1 to human IGHJ6.

63. The animal of any one of claims 55-59, wherein the animal comprises an unmodified human sequence derived from a human heavy chain immunoglobulin gene locus starting from human IGHV(III)-82 to human IGHJ6.

64. The animal of any one of claims 1-63, wherein the animal lacks an endogenous immunoglobulin heavy chain variable region locus that is capable of rearranging and forming a nucleic acid sequence that encodes an endogenous heavy chain variable domain (e.g., a mouse heavy chain variable domain).

65. The animal of any one of claims 1-63, wherein the animal lacks an endogenous immunoglobulin light chain variable region locus that is capable of rearranging and forming a nucleic acid sequence that encodes an endogenous light chain variable domain (e.g., a mouse light chain variable domain).

66. The animal of any one of claims 1-65, wherein the animal can produce a humanized antibody.

67. The animal of any one of claims 1-66, wherein the genome of the animal comprises at least one chromosome comprising a sequence encoding the fusion protein.

68. The animal of any one of claims 1-67, wherein the fusion protein comprises a human or humanized B2M protein.

69. The animal of any one of claims 1-68, wherein the MHC α chain is a MHC class I α chain.

70. The animal of any one of claims 1-69, wherein the MHC α chain is a chimeric MHC α chain.

71. The animal of any one of claims 1-69, wherein the MHC α chain is a human HLA protein (e.g., HLA-A, HLA-B, or HLA-C).

72. The animal of any one of claims 1-69, wherein the MHC α chain is a human HLA/mouse H-2 chimeric molecule, wherein the human HLA is selected from the group consisting of HLA-A, HLA-B, HLA-C, and wherein the mouse H-2 is selected from the group consisting of H-2K, H-2D, and H-2L.

73. The animal of any one of claims 1-69, wherein the MHC α chain is a human HLA/mouse H2-D1 chimeric molecule.

74. The animal of any one of claims 1-69, wherein the fusion protein comprises a human B2M protein and a chimeric MHC α chain comprising human HLA α1 and α2 domains.

75. The animal of claim 74, wherein the chimeric MHC α chain further comprises a mouse H2-D1 α3 domain.

76. The animal of any one of claims 67-75, wherein the sequence encoding the fusion protein is operably linked to an endogenous regulatory element (e.g., a promoter) at the endogenous β2 microglobulin (B2M) gene locus in the at least one chromosome.

77. The animal of any one of claims 71-76, wherein the human HLA is human HLA-A*0101, HLA-A*0201, HLA-A*0301, HLA-A*0302, HLA-A*1101, HLA-A*2402, HLA-A*2901, HLA-A*3101, HLA-A*3201, HLA-A*3301, HLA-A*3303, HLA-B*4402, HLA-B*0702, HLA-C*0702, HLA-C*0102, HLA-C*0701, HLA-C*0401, HLA-C*0801, or HLA-C*0802.

78. The animal of any one of claims 1-70, wherein the fusion protein comprises
(a) a human B2M; and
(b) a human HLA (e.g., HLA-A, HLA-B, or HLA-C).

79. The animal of claim 78, wherein the human B2M and the human HLA are linked via a linker peptide sequence.

80. The animal of any one of claims 1-70, wherein the fusion protein comprises
(a) a human B2M; and
(b) a chimeric MHC α chain.

81. The animal of claim 80, wherein the human B2M and the chimeric MHC α chain are linked via a linker peptide sequence.

82. The animal of claim 80 or 81, wherein the chimeric MHC α chain comprises human HLA α1 and α2 domains.

83. The animal of claim 82, wherein the chimeric MHC α chain further comprises a human HLA α3 domain.

84. The animal of claim 82, wherein the chimeric MHC α chain further comprises a MHC 3 domain endogenous to the animal and/or a MHC cytoplasmic region endogenous to the animal.

85. The animal of any one of claims 80-84, wherein the chimeric MHC α chain comprises a α3 domain, a connecting peptide, a transmembrane region, and a cytoplasmic region of an endogenous MHC.

86. The animal of any one of claims 80-84, wherein the animal is a mouse, and the chimeric MHC α chain comprises a α3 domain, a connecting peptide, a transmembrane region, and a cytoplasmic region of mouse H2-D1.

87. The animal of any one of claims 71-86, wherein the fusion protein further comprises a signal peptide of the human HLA (e.g., at the N-terminus of the fusion protein).

88. The animal of any one of claims 1-87, wherein the animal is heterozygous with respect to the sequence encoding the fusion protein.

89. The animal of any one of claims 1-87, wherein the animal is homozygous with respect to the sequence encoding the fusion protein.

90. A cell obtained from the animal of any one of claims 1-89.

91. The cell of claim 90, wherein the cell is a B cell that expresses a chimeric immunoglobulin heavy chain comprising an immunoglobulin heavy chain variable domain that is derived from a rearrangement of one or more human IGHV genes, one or more human IGHD genes, and one or more human IGHJ genes, wherein the immunoglobulin heavy chain variable domain is operably linked to a non-human heavy chain constant region.

92. The cell of claim 90, wherein the cell is a B cell that expresses a chimeric immunoglobulin light chain comprising an immunoglobulin light chain variable domain that is derived from a rearrangement of one or more human IGKV genes and one or more human IGKJ genes, and wherein the immunoglobulin light chain variable domain is operably linked to a non-human light chain constant region.

93. The cell of claim 90, wherein the cell is an embryonic stem (ES) cell.

94. A method of making an antibody that specifically binds to an antigen peptide-MHC complex, the method comprising
exposing the animal of any one of claims 1-89 to the antigen peptide-MHC complex comprising the antigen peptide.

95. The method of claim 94, wherein the method further comprises
producing a hybridoma from a cell collected from the animal; and
collecting or analyzing the antibody produced by the hybridoma.

96. The method of claim 94 or 95, wherein the method further comprises sequencing the genome of the hybridoma.

97. The method of any one of claims 94-96, wherein the antigen peptide is an antigen peptide (e.g., any one of the antigen peptides listed in Table 16).

98. A method of obtaining a nucleic acid that encodes an antibody binding domain that specifically binds to an antigen peptide-MHC complex, the method comprising exposing the animal of any one of claims 1-89 to the antigen peptide-MHC complex comprising the antigen peptide; and
sequencing nucleic acids encoding human heavy and light chain immunoglobulin variable regions in a cell that expresses a hybrid antibody that specifically binds to the antigen peptide-MHC complex.

99. A method of obtaining a sample, the method comprising
exposing the animal of any one of claims 1-89 to an antigen peptide-MHC complex; and
collecting the sample from the animal.

100. The method of claim 99, wherein the sample is a spleen tissue, a lymphoid tissue, spleen cell, or a B cell.

101. A method of screening an antibody that specifically binds to an antigen peptide-MHC complex, the method comprising
exposing the animal of any one of claims 1-89 to the antigen peptide-MHC complex comprising an antigen peptide of interest;
producing a hybridoma from a cell collected from the animal;
collecting or analyzing antibodies produced by the hybridoma;
incubating the antibodies with cells presenting the antigen peptide of interest, cells presenting a control antigen peptide and/or cells do not present any antigen peptide; and
determining that the antibodies can specifically bind to cells presenting the antigen peptide of interest, and optionally determining that the antibodies cannot bind to the cells presenting the control antigen peptide or the cells that do not present any antigen peptide.

102. The method of claim 101, wherein determining that the antibodies can specifically bind to cells presenting the antigen peptide of interest is determined by flow cytometry.

103. A method of screening an antibody that specifically binds to an antigen peptide-MHC complex, the method comprising
exposing the animal of any one of claims 1-89 to the antigen peptide-MHC complex comprising an antigen peptide of interest; and
sequencing nucleic acids encoding human heavy and light chain immunoglobulin variable regions in the cell that expresses an antibody that specifically binds to the antigen peptide-MHC complex.

104. The method of claim 103, the method further comprises
expressing an antibody comprising the encoding human heavy and light chain immunoglobulin variable regions;
incubating the antibody with cells presenting the antigen peptide of interest; and
determining that the antibody can specifically bind to cells presenting the antigen peptide of interest.

105. The method of claim 103, the method further comprises
incubating the antibody with cells presenting a control antigen peptide and/or cells do not present any antigen peptide; and
determining that the antibody cannot bind to the cells presenting the control antigen peptide or the cells that do not present any antigen peptide.


SEQUENCE LISTING (117 sequences)

SEQ ID NO: 1 - DNA, 3355 bp, synthetic construct
  source 1..3355: mol_type = other DNA
tttcaacctcagcctcatacttctagagccataagacataagtctttagctctcagagcttggaagtgcatgtctctttgacattcagtgacctgggattaccaaccctggaaatgctattccttgcctttgcctgagcagaggcaagctccgaccactttacagataacagaacaacatggaaactatttaactttattaaatggaaataagcaaattcgatgagcatatatgagactatcccaattttgcagaacttcttccccctacgtatgtaccattccagtttttcaattttcttatctataaacaaaaatacctaactaatataccatagatgtattaaagtacatagtgaataataatatatgagcacatacatctattaaagtgccattgaactctaaataactaatagtgttcaccccaacaattccttctatttctgatgctccttccttccgtgccctggaaaccactcacctgctttccgccactacagatgaatgtatcttttgtaagttctatgtaaacagagtcatacagagcatattcttttgtgtgacttctgcagcaatcgttattagatccatgttcttgtaccactagttgatttcttgtttgctcttgaccgtattccgcagacaggtctccctcacaatgagtgcagccattcacctgtttggattatttctagcttacagcgtttaaaaacaaagctgctgtgagcatccatgtacaagacattgtataaaatatgcatttgttttctctgcagggaacactgagcacagagaatagatctaatggtctattgtgtttaattaaaaatacttttttagacttacttattttgctttatttgtatgaacatgttggctacttgtatgtacagaccccatgtacacacctgtggatcttctgaagctggggttacagatggtttgagctactatatgaatgctagaaattgaaccccaatcttctgcaagagcaacaagtgctgttaaccactggtccatctcactagccgtaaacattttgctttaaataaaactattatatcgtttttcaaaatttttatatcatttacattggtaccaggagagcataaaattctagttcttctgaaagaatttttaccagtatttactattttgaagtgttgacagcaaaaaacctttttagtcaatcatataggcaaaaaaagaaaaaagaaaaaaaaaagaaaaagaaaagaagccactgtttaccagttaattattcccttgagagacaatagtattaaattgtcatatttgtagattgccaaagttggtccccttagacacacagggcttttgtcttttctttttaaatttatttatttgttgactgtgtatgcctgagtgcatgtgtgtgtgtgtgtgtacctgtgtgtaagtgtgtgtgtgaagttgtcaaaggtcaacttggcagacgttggttctctcctttcactatgtgggttcaggggactgaattctggctgtcaggcttggcgtcagtctcaccagcactcttgccagttaaacaataaatagttttcctttcgttttcattacccacataagtcataaatggaagtccaagaaaacacagaaatctccacatccagaaagctgaaagtacttcttcgcctctggctcttatttgagacaaggccttatggaactgtccagactggcctggaccttctggtctgaagcaatccttctctcagcttccccagtgccaggcactggaatgtgtgcctcctctcagtttccccacgatgacttatttcttactggttttaaagttggttttataagtgaccagaatagactctatctttaatgaacttaaattaagtgttattttctgtgacatagtaaatcagaaacaaaacaaaacaaaacagccatataatgttctattttcttaaagcatttcctagtacagttcaacacagtgtttagtataatttaatattaacatttcaggtctttaatgtattaaatgagtttttctttttaattagatgaagtatcttaactcacacctttatacataaaaattattcttatatttcagttacaaaaagggatagcaaaataacataaatgattatgattcttaaacattactggatcaaaatactatatgctgggtagacactgtaggattgggtctctgtttattatctcattaatgtccgtggagactattttatttttttctagtttagacacagcctccacactagagattgttaaaggtttttaaaaatatgttcaagaaatccagaagttatgttgttctccttggggaagccaaggccaaagccacttcaactgcaagcatggccatttctccatgtcctgatgccccatgccctcttcctacccttcctcactcgagctgcttgcggtggtgtctccctgtgctcacagcctgcctctgtcctggcttttggccaggggtttaacttctctactgggtccaccgctgatgtaatatacattctgagtaaaactatggcagcctttgtcttccgagccgataagcagaaaccccaagggaatgcatctcaaggtttatctcctcatccagaaaaaataatttattcggggattcacttcacttggtcacttagtttaccaatggccctgttctaagaaagtactactaagagaatggacaggaaacccagctaagccattgtccttccccaaaacccatggacagaacaaagaaaattacctaaatttttcaagggtacccgattaagttctccatccaaagctctaaaaggagaaacctgctagaagcaaggtcagaaatcctctcagttgcctttaggacaggagggtctttttacacacggaatcctgaaactgcctttgtatttctgggctgagattataaactaagggttgagttctgccagttaatgctcttaattgtcctggctttagttttcaagaatgcaaacttcaggtcctaagtccttttctgagtgggatattgtcagcaattgaataaatgaaggcggtcccaggctgaacgaccagatacaccaaactcaagagcacaccctagatagtagggcaccaagggtccagcccaggctgtttgaaatatcacgggactttataagaacatgaaactgaaaatgggaaagtccctttgtaacctagttcagcatcaacagctaggagactggtgacgacctccggatctgagtccggattggctgtgagttcaggaactatataagagcgcgcgccctggctggctctcatttcagtggctgctactcggcgcttcagtcgcggtcgcttcagtcgtcagc

SEQ ID NO: 2 - DNA, 3065 bp, synthetic construct
  source 1..3065: mol_type = other DNA
gacagagctctgcgggtacatcttagccctttcagtctctagttggtaggcactagatttaccttctggaggcttccggacactcagggaaagaaatggacagggttgtaacttcatgtaaggcaccgtcactgatgtgtcagaaggaagttgaggagcgtgagagggaacgtgggtgtctctgtcaggtggagtctagtggtagaaaatccagcttttcggtgaaatccagggcccttgaggccaaaagctcactcaaatcttgtattatatttatttctaggaaaggggaattgatgaagggggtggggatgggtgatctgcccagacaagcagttaccaaatggtggagccagaactcacctcctttaagaaggtgattgtcctacaaactcatttgatggtgaggtctggaatgtaaataatgtaatgttcggctaaccttctccttctctccctcccttccccttttcttttcagctctgaagattcatttgaacctgcttaattacaaatccagtttctaatatgctatacaatttatgcacgcagaaagaaatagcaatgtacacatcaccttctttatatcttactttaaatattttatgcatgttttcaaaaaattggaaatatcctagatagctgagcaataaatcttcaataagtattttgatcagaataataaatataattttaagaacaatagttgatcatatgccaaaccctctgtacttctcattacttggatgcagttactcatctttggtctatcacaacataagtgacatactttccttttggtaaagcaaagaggcctaattgaagtctgtcactgtgcccaatgcttagcaattctcacccccaaccctgtggctacttctgcttttgttacttttactaaaaataaaaaactaaaaaaaaaaaaaaaaattgttgccagacattgccagtgttacatgaagtggtttctaggtagcataaatgcctgcaagcacgtgcgtatttttcatcatctaaagaggcagtgggtatcttagaaggttcttgcagaagcaggctcctcaactaagggtcgagaggaaagtgactaggaaaggcctccaaagcagagcagtaggaggtggtgatgaaggtcagggaataataggaagaaactgagaggggagtgaggatctcagacgtgctggtcccacagggagctctagagcagagatcacatcttacagtttgtcttggtgatgtgcacagcttgttgctggctccacaggactgtatgctaggatgaaagcccagatactttggggtgcaggttatggttatggggtgcaagttatggttattggtggcaaattatgcaaaagctagggactggttcttattgctggatgtaggtagagcatgggtgactactgtgtttcctgttaaattctgctgggtctacagcacacagtctgaaaaccccactctgagttcagagttttggctaatgttaaagagggaatgctaatcaatttggaactaagtttgaatcctaatcttacaaaagttactataacatccatgacttgagctcttaattttgagagtttaattgtgtccccaaagctccttttgtggctggagatctagtccctgggggtggtgataatgagaagtggtgagggctctaagaaggagtgtggtgggaggtcctgtcaagggacaaggagccgtctgagttctgagagggctgttagaaaggcagcaagcaaccgggcggtggtggcacacgcctttaatcccagcacttgggaggcagaggcaggtggatttctgagttcgaggccagcctggtctacagagtgagttccaggacagccagggctatacaaagaaaccctgtctcaaaaaacccaaaaaaaaaaaaaaaaaaaggaaggcagcaagcctggcctcttctgcccttcctgaggcacgtcttctgttgctgtcggtgggaaagaggaacctttggttatcattaacattttgaaatttccaaactcttaagtaaatctcttcttactaagtagcttgtatcaggtattttgttataacaattactgattaatagtaaccataagccagaagtgggacaatcaatcttgcatcacaaagctttatttatttatttatatttcacacaactgttattattgtaaatttagattcatttaattttgtgtatgatacatgtacatatgtacactacatgcatgcttactgcctatgaagttcagaagaggtcatctggaggaagtcacatcccctggaactagagagttatggatgactgtgagccaccatgtgggtgctgggaactcaaccggggtcctctggaagagcagccagtacttttaactgctaagccttccagttatttttttgtgtgtatacgtttgtgtgtgaggagggagcatgcatgtatatatgcttgtgtaagggtatgcatgcagggggtgcatgtatgttcatgtgtgtgtgcatgtgtatgtgggttgtgcatgtgttgtatggggggatatgtgtgtatgtgtgtgtgtgtgtgtgtgagagagagagtgcatgaatgtatgtatgtatgtatgtatgtatgtatgtatgtatgtatgtatgtatcagaggatgggttgctgctggatatcttctccagttgctatcactctctaccttatattttgaggcagagttacagtgaacggcaagcttggagatttagccggccatccagccccagggatcttctagttaccacctttccagtgctagggtgagaggcacatagcaccttactcaacttttctgggatgagggctgaacccaggtccttcagctccaagactgaggcacctcctaggcccccagtacatgatatttatggaatgtagatgatttatcccagcaacctcctgggataaagttccttagggtcctaggtgtattgtttgatcttcagtctcttcttctgtgaaggtttcatcctgtttggctagtgaagtttcagtaggactgaaagtggctgtgttactactgggcttaaggtgcat

SEQ ID NO: 3 - DNA, 45 bp, synthetic construct
  source 1..45: mol_type = other DNA
ggaggtggcggatccggcggaggcggctcgggtggcggcggctct

SEQ ID NO: 4 - protein, 15 aa, synthetic construct
GGGGSGGGGSGGGGS

SEQ ID NO: 5 - protein, 5 aa, synthetic construct
GGGGS

SEQ ID NO: 6 - protein, 365 aa, Homo sapiens [sequence omitted]

SEQ ID NO: 7 - protein, 365 aa, Homo sapiens
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 8 - protein, 365 aa, Homo sapiens [sequence omitted]

SEQ ID NO: 9 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 10 - protein, 365 aa, Homo sapiens [sequence omitted]

SEQ ID NO: 11 - protein, 365 aa, Homo sapiens [sequence omitted]

SEQ ID NO: 12 - protein, 365 aa, Homo sapiens [sequence omitted]

SEQ ID NO: 13 - protein, 365 aa, Homo sapiens
MAVMAPRTLLLLLLGALALTQTWAGSHSMRYFFTSVSRPGRGEPRFIAVGYVDDTQFVRFDSDAASQRMEPRAPWIEQEGPEYWDQETRNVKAHSQTDRESLRIALRYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAMFAGAVVAAVRWRRKSSDRKGGSYSQAASSDSAQGSDMSLTACKV

SEQ ID NO: 14 - protein, 365 aa, Homo sapiens [sequence omitted]

SEQ ID NO: 15 - protein, 366 aa, Homo sapiens [sequence omitted]

SEQ ID NO: 16 - DNA, 25 bp, synthetic construct
  source 1..25: mol_type = other DNA
agttcagcatcaacagctaggagac

SEQ ID NO: 17 - DNA, 20 bp, synthetic construct
  source 1..20: mol_type = other DNA
tccaccctgtagcctcaaag

SEQ ID NO: 18 - DNA, 22 bp, synthetic construct
  source 1..22: mol_type = other DNA
agtgggggtgaattcagtgtag

SEQ ID NO: 19 - DNA, 25 bp, synthetic construct
  source 1..25: mol_type = other DNA
atgaggtctttttgtgggcagagca

SEQ ID NO: 20 - DNA, 25 bp, synthetic construct
  source 1..25: mol_type = other DNA
ctccctacggccacatcaccattac

SEQ ID NO: 21 - DNA, 24 bp, synthetic construct
  source 1..24: mol_type = other DNA
taacttcatgtaaggcaccgtcac

SEQ ID NO: 22 - DNA, 23 bp, synthetic construct
  source 1..23: mol_type = other DNA
tccagacctcaccatcaaatgag

SEQ ID NO: 23 - protein, 9 aa, Homo sapiens
FMNKFIYEI

SEQ ID NO: 24 - protein, 9 aa, Homo sapiens
KTWGQYWQV

SEQ ID NO: 25 - protein, 9 aa, Homo sapiens
IMDQVPFSV

SEQ ID NO: 26 - protein, 9 aa, Homo sapiens
  SITE 9: note = A or V
YLEPGPVTX

SEQ ID NO: 27 - protein, 9 aa, Homo sapiens
IMDQVPFSV

SEQ ID NO: 28 - protein, 9 aa, Homo sapiens
ITDQVPFSV

SEQ ID NO: 29 - protein, 9 aa, Homo sapiens
VLHDDLLEA

SEQ ID NO: 30 - protein, 9 aa, Homo sapiens
GVLPALPQV

SEQ ID NO: 31 - protein, 9 aa, Homo sapiens
TMTRVLQGC

SEQ ID NO: 32 - protein, 9 aa, Homo sapiens
KIFGSLAFL

SEQ ID NO: 33 - protein, 9 aa, Homo sapiens
GADGVGKSA

SEQ ID NO: 34 - protein, 10 aa, Homo sapiens
VVVGAVGVGK

SEQ ID NO: 35 - protein, 10 aa, Homo sapiens
ILDTAGHEEY

SEQ ID NO: 36 - protein, 9 aa, Homo sapiens
FLWGPRALV

SEQ ID NO: 37 - protein, 9 aa, Homo sapiens
EADPTGHSY

SEQ ID NO: 38 - protein, 9 aa, Homo sapiens
AAGIGILTV

SEQ ID NO: 39 - protein, 9 aa, Homo sapiens
FLSELTQQL

SEQ ID NO: 40 - protein, 8 aa, Homo sapiens
LLLTVLTW

SEQ ID NO: 41 - protein, 9 aa, Homo sapiens
AIQDLCLAV

SEQ ID NO: 42 - protein, 9 aa, Homo sapiens
SLLMWITQC

SEQ ID NO: 43 - protein, 9 aa, Homo sapiens
LLGRNSFEV

SEQ ID NO: 44 - protein, 9 aa, Homo sapiens
RMPEAAPPV

SEQ ID NO: 45 - protein, 11 aa, Homo sapiens
GLAPPQHLIRV

SEQ ID NO: 46 - protein, 9 aa, Homo sapiens
HMTEVVRHC

SEQ ID NO: 47 - protein, 9 aa, Homo sapiens
VVPCEPPEV

SEQ ID NO: 48 - protein, 9 aa, Homo sapiens
YLLPAIVHI

SEQ ID NO: 49 - protein, 9 aa, Homo sapiens
VLQELNVTV

SEQ ID NO: 50 - protein, 10 aa, Homo sapiens
ALYVDSLFFL

SEQ ID NO: 51 - protein, 10 aa, Homo sapiens
ALYVDSLFFL

SEQ ID NO: 52 - protein, 9 aa, Homo sapiens
FLRNFSLML

SEQ ID NO: 53 - protein, 9 aa, Homo sapiens
ILAKFLHWL

SEQ ID NO: 54 - protein, 9 aa, Homo sapiens
RLVDDFLLV

SEQ ID NO: 55 - protein, 9 aa, Homo sapiens
RMFPNAPYL

SEQ ID NO: 56 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 57 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 58 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 59 - protein, 362 aa, Homo sapiens
MAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 60 - protein, 362 aa, Mus musculus
MGAMAPRTLLLLLAAALAPTQTRAGPHSMRYFETAVSRPGLEEPRYISVGYVDNKEFVRFDSDAENPRYEPRAPWMEQEGPEYWERETQKAKGQEQWFRVSLRNLLGYYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 61 - protein, 476 aa, synthetic construct
MAVMAPRTLVLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 62 - protein, 479 aa, synthetic construct
MAVMAPRTLVLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 63 - protein, 479 aa, synthetic construct
MAVMAPRTLLLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHVAEQQRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 64 - protein, 479 aa, synthetic construct
MAVMAPRTLLLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 65 - protein, 479 aa, synthetic construct
MAVMAPRTLVLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV

SEQ ID NO: 66 - protein, 479 aa, synthetic construct
MAVMAPRTLLLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 67 - protein, 479 aa, synthetic construct
MAVMAPRTLLLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 68 - protein, 479 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFTTSVSRPGRGEPRFIAVGYVDDTQFVRFDSDAASQRMEPRAPWIEQEGPEYWDLQTRNVKAQSQTDRANLGTLRGYYNQSEAGSHTIQMMYGCHVGSDGRFLRGYRQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVFAGAVVAAVRWRRKSSDRKGGSYSQAASSDSAQGSDMSLTACKV

SEQ ID NO: 69 - protein, 479 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFTTSVSRPGRGEPRFIAVGYVDDTQFVRFDSDAASQRMEPRAPWIEQERPEYWDQETRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVFAGAVVAAVRWRRKSSDRKGGSYSQAASSDSAQGSDMSLTACKV

SEQ ID NO: 70 - protein, 479 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAHSQTDRESLRIALRYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAMFAGAVVAAVRWRRKSSDRKGGSYSQAASSDSAQGSDMSLTACKV

SEQ ID NO: 71 - protein, 479 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRHLENGKETLQRTDPPRTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVFAGAVVAAVRWRRKSSDRKGGSYSQAASSDSAQGSDMSLTACKV

SEQ ID NO: 72 - protein, 480 aa, synthetic construct
MRVMAPRTLILLLSGALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSSQPTIPIVGIVAGLAVLAVLAVLGAVMAVVMCRRKSSGGKGGSCSQAASSNSAQGSDESLIACKA

SEQ ID NO: 73 - protein, 479 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVFAGAVVAAVRWRRKSSDRKGGSYSQAASSDSAQGSDMSLTACKV

SEQ ID NO: 74 - protein, 476 aa, synthetic construct
MRVTAPRTLLLLLWGAVALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 75 - protein, 476 aa, synthetic construct
MLVMAPRTVLLLLSAALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 76 - protein, 480 aa, synthetic construct
MRVMAPRALLLLLSGGLALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 77 - protein, 480 aa, synthetic construct
MRVMAPRTLILLLSGALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 78 - protein, 480 aa, synthetic construct
MRVMAPRALLLLLSGGLALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 79 - protein, 480 aa, synthetic construct
MRVMAPRTLILLLSGALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKPSSQPTIPIVGIVAGLAVLAVLAVLGAMVAVVMCRRKSSGGKGGSCSQAASSNSAQGSDESLIACKA

SEQ ID NO: 80 - protein, 480 aa, synthetic construct
MRVMAPRTLILLLSGALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSSQPTIPIVGIVAGLAVLAVLAVLGAVMAVVMCRRKSSGGKGGSCSQAASSNSAQGSDESLIACKA

SEQ ID NO: 81 - DNA, 112 bp, synthetic construct
  source 1..112: mol_type = other DNA
cgggtaccaggggccacggggcgcctccctgatcgcctgtagatctggatcctgtgtgacatacctgtaccttgtcctccagagtcaggggctgggagtcattttctctggc

SEQ ID NO: 82 - DNA, 113 bp, synthetic construct
  source 1..113: mol_type = other DNA
cattcaactgacatttgttgtctgctagggatatgactgcttttgcggccgcgacagagctctgcgggtacatcttagccctttcagtctctagttggtaggcactagattta

SEQ ID NO: 83 - DNA, 82 bp, synthetic construct
  source 1..82: mol_type = other DNA
ctcgggggccctggccctgacccagacctgggcaatccagcgtactccaaagattcaggtttactcacgtcatccagcagag

SEQ ID NO: 84 - DNA, 137 bp, synthetic construct
  source 1..137: mol_type = other DNA
catgtgactttgtcacagcccaagatagttaagtgggatcgagacatgggaggtggcggatccggcggaggcggctcgggtggcggcggctctggctcccactccatgaggtatttctccacatccgtgtcccggcc

SEQ ID NO: 85 - DNA, 144 bp, synthetic construct
  source 1..144: mol_type = other DNA
gctgctccattgtccttggcccttcagtagaaccttgtcccaccaagacccgtgattacgacagagctctgcgggtacatcttagccctttcagtctctagttggtaggcactagatttaccttctggaggcttccggacactc

SEQ ID NO: 86 - DNA, 83 bp, synthetic construct
  source 1..83: mol_type = other DNA
cctgctactctcgggggccctggccctgacccagacctgggcgatccagcgtactccaaagattcaggtttactcacgtcatc

SEQ ID NO: 87 - DNA, 131 bp, synthetic construct
  source 1..131: mol_type = other DNA
gactttgtcacagcccaagatagttaagtgggatcgagacatgggaggtggcggatccggcggaggcggctcgggtggcggcggctctggctcccactccatgaggtatttctacacctccgtgtcccggc

SEQ ID NO: 88 - DNA, 127 bp, synthetic construct
  source 1..127: mol_type = other DNA
cagtttctgttccggccaccatgggctttatgtggtcacagctcacctgggtcatctttgctgctccatgacagagctctgcgggtacatcttagccctttcagtctctagttggtaggcactagat

SEQ ID NO: 89 - DNA, 73 bp, synthetic construct
  source 1..73: mol_type = other DNA
ggccctggccctgacccagacctgggcgatccagcgtactccaaagattcaggtttactcacgtcatccagca

SEQ ID NO: 90 - DNA, 126 bp, synthetic construct
  source 1..126: mol_type = other DNA
gtcacagcccaagatagttaagtgggatcgagacatgggaggtggcggatccggcggaggcggctcgggtggcggcggctctggctcccactccatgaggtatttcttcacatccgtgtcccggcc

SEQ ID NO: 91 - DNA, 123 bp, synthetic construct
  source 1..123: mol_type = other DNA
gaatagggaggtgctctacagtgatcattcattcaactgacatttgttgtctgctagggagacagagctctgcgggtacatcttagccctttcagtctctagttggtaggcactagatttacc

SEQ ID NO: 92 - protein, 365 aa, Homo sapiens [sequence omitted]

SEQ ID NO: 93 - protein, 362 aa, Homo sapiens
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 94 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 95 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 96 - protein, 366 aa, Homo sapiens
MRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 97 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 98 - protein, 366 aa, Homo sapiens
MRVMAPRTLILLLSGALALTETWAGSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKPSSQPTIPIVGIVAGLAVLAVLAVLGAMVAVVMCRRKSSGGKGGSCSQAASSNSAQGSDESLIACKA

SEQ ID NO: 99 - protein, 366 aa, Homo sapiens [sequence omitted]

SEQ ID NO: 100 - protein, 476 aa, synthetic construct
MAVMAPRTLLLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHVAEQQRAYLDGTCVEWLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 101 - protein, 476 aa, synthetic construct
MAVMAPRTLLLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 102 - protein, 476 aa, synthetic construct
MAVMAPRTLVLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 103 - protein, 476 aa, synthetic construct
MAVMAPRTLLLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 104 - protein, 476 aa, synthetic construct
MAVMAPRTLLLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 105 - protein, 476 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFTTSVSRPGRGEPRFIAVGYVDDTQFVRFDSDAASQRMEPRAPWIEQEGPEYWDLQTRNVKAQSQTDRANLGTLRGYYNQSEAGSHTIQMMYGCHVGSDGRFLRGYRQDAYDGKDYIALNEDLRSWTAADMAAQITQRKWEAARVAEQLRAYLEGTCVEWLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 106 - protein, 476 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFTTSVSRPGRGEPRFIAVGYVDDTQFVRFDSDAASQRMEPRAPWIEQERPEYWDQETRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 107 - protein, 476 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAHSQTDRESLRIALRYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 108 - protein, 476 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRHLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 109 - protein, 476 aa, synthetic construct
MRVMAPRTLILLLSGALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRADSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 110 - protein, 476 aa, synthetic construct
MAVMAPRTLLLLLLGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 111 - protein, 476 aa, synthetic construct
MRVTAPRTLLLLLWGAVALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 112 - protein, 476 aa, synthetic construct
MLVMAPRTVLLLLSAALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 113 - protein, 476 aa, synthetic construct
MRVMAPRALLLLLSGGLALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRADSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 114 - protein, 476 aa, synthetic construct
MRVMAPRTLILLLSGALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRADSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 115 - protein, 476 aa, synthetic construct
MRVMAPRALLLLLSGGLALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRADSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 116 - protein, 476 aa, synthetic construct
MRVMAPRTLILLLSGALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRADSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 117 - protein, 476 aa, synthetic construct
MRVMAPRTLILLLSGALALTETWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRADSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA